(19) United States
(12) Patent Application Publication
(10) Pub. No.: US 2011/0201003 A1
(43) Pub. Date: Aug. 18, 2011

(54) METHOD FOR OBTAINING PURKINJE PROGENITOR CELL BY USING NEPH3(65B13) AND E-CADHERIN

(75) Inventors: Yuichi Ono, Hyogo (JP); Yasuko Nakagawa, Hyogo (JP); Eri Mizuhara, Hyogo (JP)

(73) Assignee: Eisai R&D Management Co., Ltd., Tokyo (JP)

(21) Appl. No.: 13/057,838

(22) PCT Filed: Aug. 6, 2009
(86) PCT No.: PCT/JP2009/063915

§ 371 Date: Feb. 7, 2011

(30) Foreign Application Priority Data
Aug. 8, 2008 (JP) 2008-206309

(51) Int. Cl. C12N 5/0797; G01N 33/566; C12Q 1/68; C07H 21/00; C07K 16/18

(52) U.S. Cl. 435/6.11; 435/7.1; 536/24.33; 435/325; 536/24.31; 530/387.9; 536/23.5; 536/23.1; 435/6.1

(57) ABSTRACT

65B13 was discovered to be a useful marker for isolating GABA neuron progenitor cells including Purkinje cells. Furthermore, E-cadherin was revealed to be a useful marker for isolating Purkinje progenitor cells from a 65B13-positive cell population. Specifically, when used in combination with 65B13, E-cadherin was found to be a useful marker for isolating Purkinje progenitor cells.

### CROSS-REFERENCES TO RELATED APPLICATIONS

The present application is a U.S. National Phase of PCT/JP2009/063915, filed Aug. 6, 2009, which claims the benefit of Japanese Application No. 2008-206309, filed on Aug. 8, 2008, the disclosures of which are hereby incorporated herein by reference in their entirety.

### TECHNICAL FIELD

The present invention provides a marker gene and protein for Purkinje progenitor cells, and relates to the use of the gene and protein in identifying Purkinje progenitor cells.

### BACKGROUND ART

The brain functions by forming a complex network from a great variety of neurons. Its failure may result in various neurological diseases. To treat such diseases, transplantation and regeneration therapies are currently investigated. The most important thing in these therapeutic methods is to correctly identify various types of neurons in transplantation materials. Furthermore, from the viewpoint of improving safety and therapeutic effect, it is desirable to isolate only the type of cells that are needed for transplantation.

The cerebellum works on smooth motor functions such as regulation of balance, posture, and voluntary movement. The failure of cerebellar function due to cerebellar tumor, cerebellar vermis degeneration caused by chronic alcoholism, spinocerebellar degeneration, or such results in dynamic ataxia and balance disorder. Functional recovery can be achieved by replenishing lost neurons and reconstituting the network. There are about five types of neurons in the cerebellum including Purkinje cell, and formation of a proper network of the respective neurons according to organogenic program enables neurotransmission.

Research on the development of cerebellar Purkinje cells has advanced to a close understanding of their origin. However, there are few established markers for identifying progenitor cells. Cell surface markers remain unidentified, and there is no established technique for isolating viable progenitor cells.

The Neph3 gene (hereinafter sometimes referred to as “65B13”) is known to be transiently expressed in dopamine-producing neuron progenitor cells after termination of cell division (see Patent Document 1); however, there is no report published on connection of the gene with Purkinje cell. Meanwhile, it has been reported that the types of spinal cord interneurons and Purkinje cells can be identified by using the expression of the Cor11 gene and Cor12 gene as an indicator, respectively (see Patent Documents 2 and 3). At the same time, there are known methods for inducing Purkinje cells from ES cells, but they are not useful practically (Non-Patent Documents 1 and 2).

### PRIOR ART DOCUMENTS

### Patent Documents

Patent Document 1: WO 2004/038018
Patent Document 2: WO 2006/022243
Patent Document 3: WO 2006/082826

### Non-Patent Documents

Non-Patent Document 1: Su H L, Muguruma K, Matsuo-Takasaki M, Kengaku M, Watanabe K, Sasai Y. Generation of cerebellar neuron precursors from embryonic stem cells. Dev Biol. 2006 Feb. 15; 290(2):287-96.
Non-Patent Document 2: Salero E, Hatten M E. Differentiation of ES cells into cerebellar neurons. Proc Natl Acad Sci USA. 2007 Feb. 20; 104(8):2997-3002.

### DISCLOSURE OF THE INVENTION

### Problems to be Solved by the Invention

The present invention was achieved in view of the above circumstances. An objective of the present invention is to provide markers that enable selective identification of Purkinje progenitor cells. Another objective of the present invention is to provide methods of using the markers as an indicator to identify Purkinje progenitor cells, and reagents for use in these methods.

### Means for Solving the Problems

The present inventors identified a selective marker, 65B13, for GABA neuron progenitor cells in the cerebellum, and successfully isolated GABA neuron progenitor cells using antibodies that bind to the protein encoded by the gene. Specifically, 65B13 was discovered to be useful as a marker to isolate GABA-producing neuron progenitor cells in the cerebellum.

Of GABA-producing neurons, only Purkinje cells transmit neural signals outwardly from the cerebellar cortex. Degeneration of Purkinje cells is involved in the onset of spinocerebellar degeneration and such. 65B13 is a useful marker for isolating GABA neuron progenitor cells including Purkinje cells. However, to obtain high-purity Purkinje progenitor cells, Purkinje progenitor cells have to be further purified from a 65B13-positive cell population using a marker selectively expressed in Purkinje progenitor cells.

The present inventors conducted dedicated studies, and as a result revealed that E-cadherin is a useful marker for isolating Purkinje progenitor cells from a 65B13-positive cell population, thereby completing the present invention.

Specifically, E-cadherin was demonstrated to be a useful marker for isolating Purkinje progenitor cells when used in combination with 65B13.

The present invention provides markers that enable selective detection of Purkinje progenitor cells, methods of detecting Purkinje progenitor cells using the markers as an indicator, cell populations detected by the methods, and reagents for use in the methods. More specifically, the present invention provides:

[1] a method for detecting or selecting a Purkinje progenitor cell, which comprises the steps of:
(A) detecting a protein selected from:
(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and
(B) detecting a protein selected from:
(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53;
[2] a method for detecting or selecting a Purkinje progenitor cell, which comprises the step of detecting a marker protein by linking a polynucleotide encoding the marker protein to a promoter that controls (induces) expression of an mRNA translated into a protein selected from:
(A) protein selected from:
(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and
(B) a protein selected from:
(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53;
[3] the method of [1] or [2], wherein the step of detecting the protein comprises the steps of:
(d) contacting a test cell sample with an antibody that binds to the protein described in [1] or [2]; and
(e) detecting reactivity;
[4] the method of any one of [1] to [3], wherein the detection step is followed by the step of separating a Purkinje progenitor cell from the detected sample;
[5] a method for detecting or selecting a Purkinje progenitor cell, which comprises the steps of:
(A) detecting the expression of a polynucleotide capable of hybridizing to a polynucleotide selected from (i), (ii), (iii), and (iv) below, or a complementary sequence thereof:
(i) a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(ii) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iii) a polynucleotide which is expressed in a Purkinje progenitor cell and hybridizes under stringent conditions to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iv) a polynucleotide which is expressed in a Purkinje progenitor cell and has 80% or higher sequence identity to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(B) detecting the expression of a polynucleotide capable of hybridizing to a polynucleotide selected from (i′), (ii′), (iii′), and (iv′) below, or a complementary sequence thereof:
(i′) a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(ii′) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(iii′) a polynucleotide which is expressed in a Purkinje progenitor cell and hybridizes under stringent conditions to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
(iv′) a polynucleotide which is expressed in a Purkinje progenitor cell and has 80% or higher sequence identity to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
[6] the method of [5], wherein the step of detecting the expression of a polynucleotide comprises the steps of:
(a) contacting a test cell sample with a polynucleotide that can hybridize to the polynucleotide described in [5] or to a complementary sequence thereof, or with a probe comprising the polynucleotide; and
(b) detecting reactivity;
[7] the method of [6], wherein the probe is contacted with mRNA prepared from the test cell sample or a complementary DNA (cDNA) transcribed from the mRNA in step (a);
[8] the method of [5], wherein the step of detecting the expression of a polynucleotide that can hybridize to the polynucleotide described in [5] or to a complementary sequence thereof comprises the steps of:
(a-1) conducting gene amplification using a polynucleotide derived from the test cell sample as a template, and a primer comprising a polynucleotide that can hybridize to the polynucleotide described in [5] or to a complementary sequence thereof, or a set of primers comprising a polynucleotide that can hybridize to the polynucleotide selected in [5] or to a complementary sequence thereof; and
(b-1) detecting the resulting amplification product;
[9] the method of [8], wherein mRNA prepared from the test cell sample or a complementary DNA (cDNA) transcribed from the mRNA is used as a template in step (a-1);
[10] the method of any one of [5] to [9], wherein the detection step is followed by the step of separating a Purkinje progenitor cell from the detected sample;
[11] a kit for detecting a Purkinje progenitor cell, which comprises a probe, a primer, or a set of primers that enable detection of the expression of or selection of a polynucleotide that can hybridize to a polynucleotide selected from (A) and (B) below, or to a complementary sequence thereof:

### (A)

(i) a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(ii) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iii) a polynucleotide which is expressed in a Purkinje progenitor cell and hybridizes under stringent conditions to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iv) a polynucleotide which is expressed in a Purkinje progenitor cell and has 80% or higher sequence identity to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;

### (B)

(i′) a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(ii′) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(iii′) a polynucleotide which is expressed in a Purkinje progenitor cell and hybridizes under stringent conditions to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
(iv′) a polynucleotide which is expressed in a Purkinje progenitor cell and has 80% or higher sequence identity to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
[12] a kit for detecting or selecting a Purkinje progenitor cell, which comprises antibodies that bind to proteins described in:
(A) a protein selected from:
(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and
(B) a protein selected from:
(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53;
[13] a kit for detecting or selecting a Purkinje progenitor cell, which comprises a polynucleotide comprising a polynucleotide encoding a marker protein linked to a promoter that controls (induces) expression of an mRNA translated into a protein selected from:

### (A)

(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and

### (B)

(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53;
[14] a method for producing a Purkinje progenitor cell population, which comprises the steps of:
(VII) obtaining a cell population potentially containing a Purkinje progenitor cell;
(VIII) detecting a Purkinje progenitor cell using the method of any one of [1] to [10]; and
(IX) growing the cell detected or selected in step (VIII);
[15] the production method of [14], wherein the Purkinje progenitor cell is used to treat cerebellar degeneration;
[16] a Purkinje progenitor cell population obtained by the steps of:
(VII) obtaining a cell population potentially containing a Purkinje progenitor cell;
(VIII) detecting or selecting a Purkinje progenitor cell using the method of any one of [1] to [10]; and
(IX) growing the cell detected in step (VIII);
[17] a reagent for detecting or selecting a Purkinje progenitor cell, which comprises a probe, a primer, or a set of primers that enable to detect the expression of a polynucleotide that can hybridize to a polynucleotide selected from (A) and (B) below, or to a complementary sequence thereof:

### (A)

(i) a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(ii) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iii) a polynucleotide which is expressed in a Purkinje progenitor cell and hybridizes under stringent conditions to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iv) a polynucleotide which is expressed in a Purkinje progenitor cell and has 80% or higher sequence identity to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;

### (B)

(i′) a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(ii′) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(iii′) a polynucleotide which is expressed in a Purkinje progenitor cell and hybridizes under stringent conditions to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
(iv′) a polynucleotide which is expressed in a Purkinje progenitor cell and has 80% or higher sequence identity to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
[18] a reagent for detecting or selecting a Purkinje progenitor cell, which comprises an antibody that binds to a protein selected from:

### (A)

(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and

### (B)

(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53;
[19] a reagent for detecting or selecting a Purkinje progenitor cell, which comprises a polynucleotide comprising a polynucleotide encoding a marker protein linked to a promoter that controls (induces) expression of an mRNA translated into a protein selected from:

### (A)

(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and

### (B)

(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53;
[20] a polynucleotide for detecting or selecting a Purkinje progenitor cell for use in regeneration medicine to treat cerebellar degeneration, which can hybridize to a polynucleotide selected from (A) and (B) below, or to a complementary sequence thereof:

### (A)

(i) a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(ii) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iii) a polynucleotide which is expressed in a Purkinje progenitor cell and hybridizes under stringent conditions to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iv) a polynucleotide which is expressed in a Purkinje progenitor cell and has 80% or higher sequence identity to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;

### (B)

(i′) a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(ii′) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(iii′) a polynucleotide which is expressed in a Purkinje progenitor cell and hybridizes under stringent conditions to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
(iv′) a polynucleotide which is expressed in a Purkinje progenitor cell and has 80% or higher sequence identity to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
[21] an antibody for detecting or selecting a Purkinje progenitor cell for use in regeneration medicine to treat cerebellar degeneration, which binds to a protein selected from:

### (A)

(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36;

### (B)

(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53.

Furthermore, the present invention relates to:

[22] a method for treating cerebellar degeneration, which comprises transplanting a Purkinje progenitor cell detected or selected by using a polynucleotide that can hybridize to a polynucleotide selected from (A), and (B) below, or to a complementary sequence thereof:

### (A)

(i) a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(ii) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iii) a polynucleotide which is expressed in a Purkinje progenitor cell and hybridizes under stringent conditions to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iv) a polynucleotide which is expressed in a Purkinje progenitor cell and has 80% or higher sequence identity to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35; and

### (B)

(i′) a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(ii′) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(iii′) a polynucleotide which is expressed in a Purkinje progenitor cell and hybridizes under stringent conditions to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
(iv′) a polynucleotide which is expressed in a Purkinje progenitor cell and has 80% or higher sequence identity to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
[23] the method of [22] for transplanting a Purkinje progenitor cell, wherein the Purkinje progenitor cell is a spinal cord or cerebellar Purkinje progenitor cell;
[24] the method of [22] or [23] for transplanting a Purkinje progenitor cell, wherein the polynucleotide comprises at least 10 consecutive nucleotides of a polynucleotide selected from (A) and (B) mentioned above, or a complementary sequence thereof;
[25] the method of [22] or [23] for transplanting a Purkinje progenitor cell, wherein the polynucleotide comprises at least 15 consecutive nucleotides of a polynucleotide selected from (A) and (B) mentioned above, or a complementary sequence thereof; and
[26] a method for treating cerebellar degeneration, which comprises transplanting a Purkinje progenitor cell detected or selected by using an antibody that binds to a protein selected from:

### (A)

(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and

### (B)

(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53;
[27] the method of [26] for transplanting a Purkinje progenitor cell, wherein the Purkinje progenitor cell is a spinal cord or cerebellar Purkinje progenitor cell; and
[28] the method of [26] or [27] for transplanting a Purkinje progenitor cell, wherein the antibody binds to a protein selected from:

### (A)

(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and

### (B)

(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53.

### Effects of the Invention

The present invention identified selective markers 65B13 and E-cadherin for Purkinje progenitor cells, and successfully isolated Purkinje progenitor cells by using antibodies against these markers. This technique can provide viable Purkinje progenitor cells, and is expected to be useful in preparing materials for transplantation therapy for degenerative diseases, search of specific genes, discovery of drugs targeting Purkinje cells, etc.

Highly pure Purkinje cells can be obtained by methods for preparing Purkinje progenitor cells using the markers of the present invention. Thus, the methods are applicable to, for example, drug discovery or regeneration medicine targeting spinocerebellar degeneration.

### BRIEF DESCRIPTION OF THE DRAWINGS

FIG. 1 shows the expression pattern of the 65B13 gene in the nervous system of fetal mouse. A, E12.5 sagittal section; B, E12.5 cerebellar primordium; C, E12.5 spinal cord; D, E10.5 spinal cord; E, E12.5 spinal cord; F, E14.5 spinal cord. CB, cerebellar primordium; SC, spinal cord.

FIG. 2 shows a comparison of the 65B13, Cor12, and Pax2 expressions in the fetal cerebellar primordium. FIG. 2A shows the result of expression analysis at E 12.5; FIG. 2B shows the result of expression analysis at E14.5. In FIG. 2B, the left and right photographs show the expression of 65B13 or Pax2, respectively.

FIG. 3 shows that 65B13-positive cells could be isolated from the cerebellar primordia of fetal mouse. FIG. 3A shows results for the E12.5 cerebella and isolated 65B13-positive cells. FIG. 3B shows results of the E14.5 cerebella and isolated 65B13-positive cells.

FIG. 4 shows differentiation of 65B13-positive cells isolated from the cerebellar primordia of E12.5 (A) and E14.5 (B) mice into GABA-producing Purkinje cells and GABA-producing Golgi cells, respectively.

FIG. 5 shows the structures of DNA constructs that can be used to select GABA-producing neuron progenitor cells.

FIG. 6 shows that a foreign gene (Gsh1) could be expressed specifically in GABA-producing neuron progenitor cells by using the 65B13 promoter.

FIG. 7 shows in a photograph results of RT-PCR analysis for the mRNA expression of 65B13 and E-cadherin in 65B13-positive cells of the cerebellar primordium, myelencephalon, and spinal cord. CB, cerebellar primordium; Mye, myelencephalon; SC, spinal cord.

FIG. 8 shows in photographs comparison of the expression of E-cadherin, 65B13, Cor12, and Pax2 in the cerebellar primordium and myelencephalon areas between E12.5 and E14.5 mice. CB, cerebellar primordium; Mye, myelencephalon; E-cad, E-cadherin.

FIG. 9 shows in diagrams results of flow cytometry analysis for the cell surface expression of 65B13 and E-cadherin in the cerebellar primordium, myelencephalon, and spinal cord of E12.5 mice.

FIG. 10 shows in diagrams and a photograph differentiation of Purkinje cells from the E-cadherin and 65B13-double positive cells isolated from cerebellar primordia of E12.5 mice. A: pattern obtained by flow cytometry analysis prior to isolation. P5: isolated population. B: purity analysis following the isolation. C: staining pattern of isolated cells after two days of culture.

### MODE FOR CARRYING OUT THE INVENTION

The 65B13 gene is known to be expressed transiently in dopamine-producing neuron progenitor cells after termination of cell division (WO 2004/038018); however, there is no report published on the connection of the gene with GABA neuron.

The present inventors discovered that GABA neuron progenitor cells could be isolated by using the expression of the 65B13 gene as an indicator. The present inventors also successfully demonstrated that the E-cadherin gene is a marker selectively expressed in Purkinje progenitor cells among 65B13-positive cells.

Specifically, the present invention provides methods for detecting or selecting Purkinje progenitor cells.

The present invention provides methods for detecting or selecting Purkinje progenitor cells, which comprise the steps of detecting a 65B13 protein and detecting an E-cadherin protein.

The steps of detecting a 65B13 protein and detecting an E-cadherin protein may be carried out simultaneously. Alternatively, either of the two may be followed by the other.

The 65B13 gene of the present invention includes, for example, two types of genes named 65B13-a and 65B13-b, which are alternative isoforms of the 65B13 gene. The respective nucleotide sequences are shown in SEQ ID NOs: 1 and 3, and the amino acid sequences encoded by the genes are shown in SEQ ID NOs: 2 and 4.

The coding region of 65B13-a starts with A at position 178 in SEQ ID NO: 1, and extends to the stop codon at positions 2278 to 2280, encoding a protein of 700 amino acids. The 17 amino acid residues encoded by the sequence of positions 178 to 228 constitute a signal sequence, while the 17 amino acid residues encoded by the sequence of positions 1717 to 1767 constitute a transmembrane region.

On the other hand, the coding region of 65B13-b starts with A at position 127 in SEQ ID NO: 2 and extends to the stop codon at positions 2277 to 2079, encoding a protein of 650 amino acids. The 17 amino acid residues encoded by the sequence of positions 127 to 178 constitute a signal sequence, while the 17 amino acid residues encoded by the sequence of positions 1516 to 1566 constitute a transmembrane region.

Furthermore, the 65B13 gene of the present invention also includes, for example, the genes of SEQ ID NOs: 19, 21, 23, 25, 27, 29, 31, 33, and 35. The amino acid sequences encoded by the genes are shown in SEQ ID NOs: 20, 22, 24, 26, 28, 30, 32, 34, and 36, respectively.

The coding region of SEQ ID NO: 19 starts with A at position 668 and extends to the stop codon at positions 2768 to 2770, encoding a protein of 700 amino acids. The 19 amino acid residues encoded by the sequence of positions 668 to 724 constitute a signal sequence, while the 494 amino acid residues encoded by the sequence of positions 725 to 2206 constitute an extracellular domain.

The coding region of SEQ ID NO: 21 starts with A at position 130 and extends to the stop codon at positions 2230 to 2232, encoding a protein of 700 amino acids. The 19 amino acid residues encoded by the sequence of positions 130 to 186 constitute a signal sequence, while the 494 amino acid residues encoded by the sequence of positions 187 to 1668 constitute an extracellular domain.

The coding region of SEQ ID NO: 23 starts with A at position 199 and extends to the stop codon at positions 2098 to 2100, encoding a protein of 633 amino acids. The 20 amino acid residues encoded by the sequence of positions 199 to 258 constitute a signal sequence, while the 490 amino acid residues encoded by the sequence of positions 259 to 1728 constitute an extracellular domain.

The coding region of SEQ ID NO: 25 starts with A at position 199 and extends to the stop codon at positions 2323 to 2325, encoding a protein of 708 amino acids. The 20 amino acid residues encoded by the sequence of positions 199 to 258 constitute a signal sequence, while the 490 amino acid residues encoded by the sequence of positions 259 to 1728 constitute an extracellular domain.

The coding region of SEQ ID NO: 27 starts with A at position 199 and extends to the stop codon at positions 2323 to 2325, encoding a protein of 708 amino acids. The 20 amino acid residues encoded by the sequence of positions 199 to 258 constitute a signal sequence, while the 490 amino acid residues encoded by the sequence of positions 259 to 1728 constitute an extracellular domain.

The coding region of SEQ ID NO: 29 starts with A at position 15 and extends to the stop codon at positions 1914 to 1916, encoding a protein of 633 amino acids. The 20 amino acid residues encoded by the sequence of positions 15 to 74 constitute a signal sequence, while the 490 amino acid residues encoded by the sequence of positions 75 to 1544 constitute an extracellular domain.

The coding region of SEQ ID NO: 31 starts with A at position 199 and extends to the stop codon at positions 1948 to 1950, encoding a protein of 583 amino acids. The 20 amino acid residues encoded by the sequence of positions 199 to 258 constitute a signal sequence, while the 440 amino acid residues encoded by the sequence of positions 259 to 1578 constitute an extracellular domain.

The coding region of SEQ ID NO: 33 starts with A at position 15 and extends to the stop codon at positions 1764 to 1766, encoding a protein of 583 amino acids. The 20 amino acid residues encoded by the sequence of positions 15 to 74 constitute a signal sequence, while the 440 amino acid residues encoded by the sequence of positions 75 to 1394 constitute an extracellular domain.

The coding region of SEQ ID NO: 35 starts with A at position 196 and extends to the stop codon at positions 2260 to 2262, encoding a protein of 688 amino acids. The 20 amino acid residues encoded by the sequence of positions 196 to 255 constitute a signal sequence, while the 470 amino acid residues encoded by the sequence of positions 256 to 1665 constitute an extracellular domain.

In addition, the 65B13 gene of the present invention also includes, for example, the sequences of accession numbers XM_{—}994164, AL136654, XM_{—}512603, XR_{—}012248, XM_{—}541684, and XM_{—}583222.

The coding region of SEQ ID NO: 46 starts with A at position 125 and extends to the stop codon at positions 2771 to 2773, encoding a protein of 883 amino acids. The 22 amino acid residues encoded by the sequence of positions 125 to 190 constitute a signal sequence, while the 686 amino acid residues encoded by the sequence of positions 191 to 2248 constitute an extracellular domain.

The coding region of SEQ ID NO: 48 starts with A at position 128 and extends to the stop codon at positions 2780 to 2782, encoding a protein of 885 amino acids. The 22 amino acid residues encoded by the sequence of positions 128 to 193 constitute a signal sequence, while the 688 amino acid residues encoded by the sequence of positions 194 to 2257 constitute an extracellular domain.

The coding region of SEQ ID NO: 50 starts with A at position 127 and extends to the stop codon at positions 2785 to 2787, encoding a protein of 887 amino acids. The 22 amino acid residues encoded by the sequence of positions 127 to 192 constitute a signal sequence, while the 690 amino acid residues encoded by the sequence of positions 193 to 2262 constitute an extracellular domain.

The coding region of SEQ ID NO: 52 starts with A at position 192 and extends to the stop codon at positions 2838 to 2840, encoding a protein of 883 amino acids. The 22 amino acid residues encoded by the sequence of positions 192 to 257 constitute a signal sequence, while the 686 amino acid residues encoded by the sequence of positions 258 to 1933 constitute an extracellular domain.

A preferred embodiment of the method for detecting or selecting a Purkinje progenitor cell comprises the steps of:

(A) detecting a protein selected from:
(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and
(B) detecting a protein selected from:
(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53.

A “protein” in the present invention can also be referred to as a “polypeptide” in general. A “polypeptide” of the present invention refers to a peptide polymer encoded by a polynucleotide of the present invention. In the present invention, proteins having 65B13 protein or E-cadherin protein can be included. Preferred examples include proteins having the amino acid sequence described in SEQ ID NO: 2, 4, 20, 22, 24, 26, 28, 30, 32, 34, or 36, or proteins having the amino acid sequence described in SEQ ID NO: 47, 49, 51, or 53.

The polypeptides of the present invention may comprise naturally occurring or modified amino acid residues. Examples of amino acid residue modifications include acylation, acetylation, amidation, arginylation, GPI anchor formation, crosslinking, y-carboxylation, cyclization, covalent crosslink formation, glycosylation, oxidation, covalent bonding of a lipid or fat derivative, cystine formation, disulfide bond formation, selenoylation, demethylation, protein fragmentation treatment, covalent bonding of a nucleotide or nucleotide derivative, hydroxylation, pyroglutamate formation, covalent bonding of a flavin, prenylation, covalent bonding with a heme portion, covalent bonding of phosphatidyl inositol, formylation, myristoylation, methylation, ubiquitination, iodination, racemization, ADP-ribosylation, sulfation, and phosphorylation.

Moreover, the polypeptides of the present invention include precursors containing a signal peptide portion, mature proteins lacking a signal peptide portion, and fusion proteins modified with other peptide sequences. Peptide sequences to be added to a polypeptide of the present invention can be selected from sequences that facilitate protein purification using, for example, pcDNA3.1/Myc-His vector (Invitrogen), or those that confer stability in recombinant protein production. Examples of such sequences are influenza agglutinin (HA), glutathione S transferase (GST), substance P, multiple histidine tag (such as 6×His and 10×His), protein C fragment, maltose-binding protein (MBP), immunoglobulin constant region, α-tubulin fragment, 3-galactosidase, B-tag, c-myc fragment, E-tag (epitope on a monoclonal phage), FLAG (Hopp et al. Bio/Technol. 1988, 6:1204-10), lck tag, p18 HIV fragment, HSV-tag (human simple Herpes virus glycoprotein), SV40T antigen fragment, T7-tag (T7 gene 10 protein), and VSV-GP fragment (vesicular stomatitis virus glycoprotein).

It is well known that a mutant polypeptide comprising an amino acid sequence, in which one or more amino acids are deleted, inserted, substituted, or added, maintains the same biological activity as the original polypeptide (Mark et al. Proc. Natl. Acad. Sci. USA 1984, 81:5662-6; Zoller and Smith. Nucleic Acids Res. 1982, 10:6487-500; Wang et al. Science 1984, 224:1431-3; Dalbadie-McFarland et al. Proc. Natl. Acad. Sci. USA 1982, 79:6409-13). Such mutant polypeptides may be those, for example, having an insertion, substitution, or deletion of 1 to 30 amino acids, preferably 1 to 20 amino acids, more preferably 1 to 10 amino acids, even more preferably 1 to 5 amino acids, and still more preferably one or two amino acids, or those that have amino acids added to either or both ends. The mutant polypeptides may be those having an amino acid sequence preferably with one or more conservative amino acid substitutions (preferably one or several substitutions, more preferably one, two, or three substitutions) in the original amino acid sequence.

Here, an amino acid substitution refers to a mutation in which one or more amino acid residues in a sequence are changed to a different type of amino acid residue. When the amino acid sequence encoded by a polynucleotide of the present invention is altered by such a substitution, a conservative substitution is preferably carried out if the function of the protein is to be maintained. A conservative substitution means altering a sequence so that it encodes an amino acid that has properties similar to those of the amino acid before substitution.

Amino acids can be classified, based on their properties, into non-polar amino acids (Ala, Ile, Leu, Met, Phe, Pro, Trp, Val), non-charged amino acids (Asn, Cys, Gln, Gly, Ser, Thr, Tyr), acidic amino acids (Asp, Glu), basic amino acids (Arg, His, Lys), neutral amino acids (Ala, Asn, Cys, Gln, Gly, Ile, Leu, Met, Phe, Pro, Ser, Thr, Trp, Tyr, Val), aliphatic amino acids (Ala, Gly), branched amino acids (Ile, Leu, Val), hydroxyamino acids (Ser, Thr), amide-type amino acids (Gln, Asn), sulfur-containing amino acids (Cys, Met), aromatic amino acids (His, Phe, Trp, Tyr), heterocyclic amino acids (His, Trp), imino acids (Pro, 4Hyp), etc. In particular, substitutions among Ala, Val, Leu, and Ile; Ser and Thr; Asp and Glu; Asn and Gln; Lys and Arg; and Phe and Tyr are preferable in order to maintain protein properties. There are no particular limitations on the number and sites of the mutated amino acids, as long as the amino acids encoded by the polynucleotide have the antigenicity of 65B13 or E-cadherin.

A polynucleotide encoding an amino acid sequence, in which one or more amino acids are deleted, inserted, substituted, or added to the amino acid sequence of SEQ ID NO: 2, 4, 20, 22, 24, 26, 28, 30, 32, 34, or 36, or the amino acid sequence of SEQ ID NO: 47, 49, 51, or 53 can be prepared according to methods such as site-directed mutagenesis described in “Molecular Cloning, A Laboratory Manual 2^{nd} ed.” (Cold Spring Harbor Press (1989)), “Current Protocols in Molecular Biology” (John Wiley & Sons (1987-1997), Sections 8.1-8.5), Hashimoto-Goto et al. (Gene 1995, 152:271-5), Kunkel (Proc. Natl. Acad. Sci. USA 1985, 82:488-92), Kramer and Fritz (Method. Enzymol. 1987, 154:350-67), Kunkel (Method. Enzymol. 1988, 85:2763-6), etc.

The above-described proteins of the present invention also include proteins encoded by polynucleotides that hybridize under stringent conditions to a polynucleotide encoding the amino acid sequence of SEQ ID NO: 2, 4, 20, 22, 24, 26, 28, 30, 32, 34, or 36, or the amino acid sequence of SEQ ID NO: 47, 49, 51, or 53 which are functionally equivalent to a protein comprising the amino acid sequence of SEQ ID NO: 2, 4, 20, 22, 24, 26, 28, 30, 32, 34, or 36, or the amino acid sequence of SEQ ID NO: 47, 49, 51, or 53.

“Equivalent function to a protein comprising the amino acid sequence of SEQ ID NO: 2, 4, 20, 22, 24, 26, 28, 30, 32, 34, or 36” preferably refers to the selective expression in GABA-producing neuron progenitor cells. “Equivalent function to a protein comprising the amino acid sequence of SEQ ID NO: 47, 49, 51, or 53” preferably refers to the selective expression in Purkinje progenitor cells.

Examples of hybridization conditions in the present invention include “2×SSC, 0.1% SDS, 50° C.”, “2×SSC, 0.1% SDS, 42° C.”, and “1×SSC, 0.1% SDS, 37° C.”. Examples of conditions of higher stringency include “2×SSC, 0.1% SDS, 65° C.”, “0.5×SSC, 0.1% SDS, 42° C.”, and “0.2×SSC, 0.1% SDS, 65° C.”. More specifically, a method that uses the Rapid-hyb buffer (Amersham Life Science) can be carried out by performing pre-hybridization at 68° C. for 30 minutes or more, adding a probe to allow hybrid formation at 68° C. for one hour or more, washing three times in 2×SSC, 0.1% SDS at room temperature for 20 minutes each, washing three times in 1×SSC, 0.1% SDS at 37° C. for 20 minutes each, and finally washing twice in 1×SSC, 0.1% SDS at 50° C. for 20 minutes each. This can also be carried out using, for example, the Expresshyb Hybridization Solution (CLONTECH), by performing pre-hybridization at 55° C. for 30 minutes or more, adding a labeled probe and incubating at 37° C. to 55° C. for one hour or more, washing three times in 2×SSC, 0.1% SDS at room temperature for 20 minutes each, and washing once at 37° C. for 20 minutes with 1×SSC, 0.1% SDS. Here, conditions of higher stringency can be achieved by increasing the temperature for pre-hybridization, hybridization, or second wash. For example, the pre-hybridization and hybridization temperature can be raised to 60° C., and to 68° C. for higher stringency. In addition to conditions such as salt concentration of the buffer and temperature, a person with ordinary skill in the art can also integrate other conditions such as probe concentration, probe length, and reaction time, to obtain 65B13 isoforms, allelic mutants, and corresponding genes derived from other organisms.

References such as “Molecular Cloning, A Laboratory Manual 2” ed.” (Cold Spring Harbor Press (1989), Sections 9.47-9.58), “Current Protocols in Molecular Biology” (John Wiley & Sons (1987-1997), Sections 6.3-6.4), “DNA Cloning 1: Core Techniques, A Practical Approach 2^{nd} ed.” (Oxford University (1995), Section 2.10 for conditions) can be referred to for detailed information on hybridization procedures. Examples of hybridizing polynucleotides include polynucleotides containing a nucleotide sequence that has at least 50% or more, preferably 70%, more preferably 80%, and even more preferably 90% (for example, 95% or more, 98% or more, or 99%) identity with a polynucleotide encoding the amino acid sequence of SEQ ID NO: 2, 4, 20, 22, 24, 26, 28, 30, 32, 34, or 36, or SEQ ID NO: 47, 49, 51, or 53. Such identities can be determined by the BLAST algorithm (Altschul. Proc. Natl. Acad. Sci. USA 1990, 87:2264-8; Karlin and Altschul. Proc. Natl. Acad. Sci. USA 1993, 90:5873-7). Examples of programs that have been developed based on this algorithm include the BLASTX program for determining the identity of amino acid sequences, and the BLASTN program for nucleotide sequences (Altschul et al. J. Mol. Biol. 1990, 215:403-10). These programs can be used for the sequences of the present invention. One can refer to, for example, http://www.ncbi.nlm.nih.gov for a specific example of analysis methods.

Alternatively, in the present invention, Purkinje progenitor cells may be detected or selected by using promoters (including modified promoters) (see, for example, Japanese Patent Application Kokai Publication No. (JP-A) 2002-51775 (unexamined, published Japanese patent application)) that control (induce) expression of an mRNA to be translated into the protein described in (A) or (B) below:

(A) a protein selected from (v), (vi), (vii), and (viii) above; and
(B) a protein selected from (v′), (vi′), (vii′), and (viii′) above.

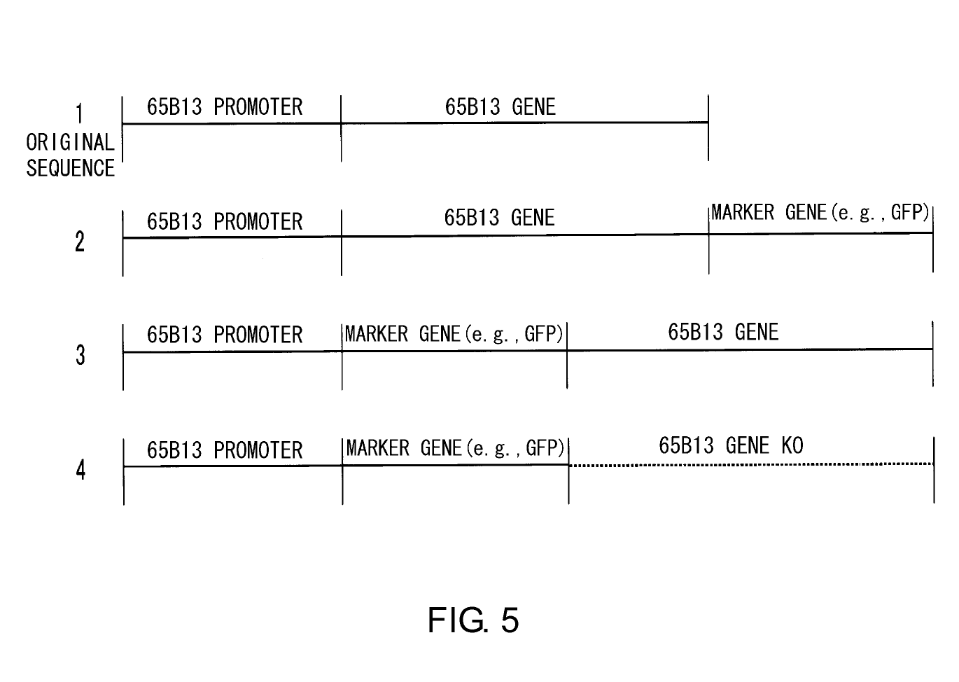

For example, it is possible to transfect cells with a vector carrying a construct in which a gene encoding a detectable marker such as GFP is linked to a promoter portion obtained by analyzing the expression regulatory region. The construct may have a structure where the gene is linked upstream or downstream of the marker gene under the control of the expression regulatory sequence (including promoters, enhancers, etc.). Alternatively, the maker gene can be knocked-in at the locus. In a preferred embodiment using 65B13, the construct includes, for example, constructs having any one of Structures 2 to 4 schematically illustrated in FIG. 5. Similar constructs are also included for E-cadherin. The expression of a marker gene specific to GABA-producing neuron progenitor cells including Purkinje cells is detected by using (A). Meanwhile, the expression of a marker gene specific to Purkinje progenitor cells is detected by using (B). The combined use of the two enables specific selection of Purkinje progenitor cells.

Specifically, the step of detecting such a target protein includes the steps of:

(d) contacting a test cell sample with an antibody that binds to the protein of (A) or (B); and
(e) detecting reactivity.

The proteins of the present invention can be detected by contacting an antibody which binds to the protein of (A) or (B) of the present invention with cell samples that may contain Purkinje progenitor cells, and detecting reactivity. The antibody may be immobilized onto appropriate carriers for use before contact with the cells. Alternatively, cells bound to the antibody can be selectively collected through affinity purification using the antibody after contacting and binding the cells with the antibody. For example, when an antibody of the present invention is linked to biotin, the cells can be purified by adding the cell sample to a plate or column immobilized with avidin or streptavidin.

The detection step may be followed by the step of isolating Purkinje progenitor cells from the detected sample. In the present invention, Purkinje progenitor cells can be efficiently separated by flow cytometry using an antibody that binds to 65B13 (anti-65B13 antibody) and an antibody that binds to E-cadherin (anti-E-cadherin antibody) which will be mentioned later.

The cell samples used in the methods are culture cells containing in vitro differentiated Purkinje cells. Purkinje cells can be differentiated in vitro by using known ES cells, iPS cells, dedifferentiated cells, or the like as a starting material. In general, Purkinje cells can be differentiated by co-culturing nerve tissue-derived supporting cell layer with brain tissues obtained from an area containing Purkinje cells. The cell sample used for selection of Purkinje progenitor cells of the present invention may be a group of cells separated or cultured by any method.

The present invention also relates to methods for detecting or selecting Purkinje progenitor cells, which comprise the steps of:

(A) detecting the expression of a polynucleotide encoding a 65B13 protein; and
(B) detecting the expression of a polynucleotide encoding an E-cadherin protein.

A preferred embodiment of the polynucleotide of step (A) mentioned above includes a polynucleotide for detecting or selecting a Purkinje progenitor cell, which is capable of hybridizing to a polynucleotide selected from (i), (ii), (iii), and (iv) below, or a complementary sequence thereof:

(i) a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(ii) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iii) a polynucleotide which is expressed in a Purkinje progenitor cell and hybridizes under stringent conditions to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iv) a polynucleotide which is expressed in a Purkinje progenitor cell and has 80% or higher sequence identity to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35.

A preferred embodiment of the polynucleotide of step (B) mentioned above includes a polynucleotide for detecting or selecting a Purkinje progenitor cell, which is capable of hybridizing to a polynucleotide selected from (i′), (ii′), (iii′), and (iv′) below, or a complementary sequence thereof:

(i′) a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(ii′) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(iii′) a polynucleotide which is expressed in a Purkinje progenitor cell and hybridizes under stringent conditions to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
(iv′) a polynucleotide which is expressed in a Purkinje progenitor cell and has 80% or higher sequence identity to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52.

In the present invention, the polynucleotides of (A) and (B) above may be referred to as “polynucleotides of the present invention”.

In the present invention, a “polynucleotide” refers to a polymer comprising nucleotides or nucleotide pairs of multiple deoxyribonucleic acids (DNA) or ribonucleic acids (RNA), and includes DNA, cDNA, genomic DNA, chemically synthesized DNA, and RNA. If needed, polynucleotides can also contain non-naturally occurring nucleotides such as 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, β-D-galactosylqueuosine, 2′-O-methylguanosine, inosine, N6-isopentenyladenosine, 1-methyladenosine, 1-methylpseudouridine, 1-methylguanosine, 1-methylinosine, 2,2-dimethylguanosine, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, β-D-mannosylqueuosine, 5-methoxycarbonylmethyl-2-thiouridine, 5-methoxycarbonylmethyluridine, 5-methoxyuridine, 2-methylthio-N6-isopentenyladenosine, N-49-β-D-ribofuranosyl-2-methylthiopurin-6-yl)carbamoyl)threonine, N-((9-β-D-ribofuranosylpurin-6-yl)N-methylcarbamoyl)threonine, uridine-5-oxyacetic acid-methyl ester, uridine-5-oxyacetic acid, wybutoxosine, pseudouridine, queuosine, 2-thiocytidine, 5-methyl-2-thiouridine, 2-thiouridine, 4-thiouridine, 5-methyluridine, N-((9-β-D-ribofuranosylpurin-6-yl)carbamoyl)threonine, 2′-O-methyl-5-methyluridine, 2′-O-methyluridine, wybutosine, and 3-(3-amino-3-carboxy propyl)uridine.

The polynucleotides of the present invention can also be produced by chemical synthesis based on the known sequences of 65B13 and E-cadherin. Alternatively, such polynucleotides can be prepared from 65B13 gene- and E-cadherin gene-expressing cells using hybridization, PCR, etc.

The polynucleotides of the present invention also include polynucleotides that hybridize under stringent conditions to a polynucleotide comprising the nucleotide sequence from positions 178 to 2280 of SEQ ID NO: 1, the nucleotide sequence from positions 127 to 2079 of SEQ ID NO: 3, the nucleotide sequence from positions 668 to 2770 of SEQ ID NO: 19, the nucleotide sequence from positions 130 to 2232 of SEQ ID NO: 21, the nucleotide sequence from positions 199 to 2100 of SEQ ID NO: 23, the nucleotide sequence from positions 199 to 2325 of SEQ ID NO: 25, the nucleotide sequence from positions 15 to 2325 of SEQ ID NO: 27, the nucleotide sequence from positions 15 to 1916 of SEQ ID NO: 29, the nucleotide sequence from positions 199 to 1950 of SEQ ID NO: 31, the nucleotide sequence from positions 15 to 1766 of SEQ ID NO: 33, or the nucleotide sequence from positions 196 to 2262 of SEQ ID NO: 35, and each of which encodes a protein that is functionally equivalent (a preferred function is the selective expression in Purkinje progenitor cells) to a protein comprising the amino acid sequence of SEQ ID NO: 2, 4, 20, 22, 24, 26, 28, 30, 32, 34, or 36. The polypeptides also include isoforms, alternative isoforms, and allelic mutants of 65B13.

Such polynucleotides can be obtained from cDNA libraries and genomic libraries of animals such as humans, mice, rats, rabbits, hamsters, chickens, pigs, cows, goats, sheep, monkeys, and dogs by known hybridization methods such as colony hybridization, plaque hybridization, and Southern blotting using as a probe a polynucleotide comprising the nucleotide sequence from positions 1 to 2876 of SEQ ID NO: 1, the nucleotide sequence from positions 1 to 2243 of SEQ ID NO: 3, the nucleotide sequence from positions 1 to 3123 of SEQ ID NO: 19, the nucleotide sequence from positions 1 to 3247 of SEQ ID NO: 21, the nucleotide sequence from positions 1 to 2153 of SEQ ID NO: 23, the nucleotide sequence from positions 1 to 2979 of SEQ ID NO: 25, the nucleotide sequence from positions 1 to 2973 of SEQ ID NO: 27, the nucleotide sequence from positions 1 to 1969 of SEQ ID NO: 29, the nucleotide sequence from positions 1 to 2003 of SEQ ID NO: 31, the nucleotide sequence from positions 1 to 1819 of SEQ ID NO: 33, or the nucleotide sequence from positions 1 to 2959 of SEQ ID NO: 35; preferably the nucleotide sequence from positions 178 to 2280 of SEQ ID NO: 1, the nucleotide sequence from positions 127 to 2079 of SEQ ID NO: 3, the nucleotide sequence from positions 668 to 2770 of SEQ ID NO: 19, the nucleotide sequence from positions 130 to 2232 of SEQ ID NO: 21, the nucleotide sequence from positions 199 to 2100 of SEQ ID NO: 23, the nucleotide sequence from positions 199 to 2325 of SEQ ID NO: 25, the nucleotide sequence from positions 15 to 2325 of SEQ ID NO: 27, the nucleotide sequence from positions 15 to 1916 of SEQ ID NO: 29, the nucleotide sequence from positions 199 to 1950 of SEQ ID NO: 31, the nucleotide sequence from positions 15 to 1766 of SEQ ID NO: 33, or the nucleotide sequence from positions 196 to 2262 of SEQ ID NO: 35. Regarding methods for constructing cDNA libraries, one can refer to “Molecular Cloning, A Laboratory Manual 2^{nd} ed.” (Cold Spring Harbor Press (1989)). It is also possible to use cDNA libraries and genomic libraries available on the market.

The polynucleotides of the present invention also include polynucleotides that hybridize under stringent conditions to a polynucleotide comprising the nucleotide sequence from positions 125 to 2773 of SEQ ID NO: 46, the nucleotide sequence from positions 128 to 2782 of SEQ ID NO: 48, the nucleotide sequence from positions 127 to 2787 of SEQ ID NO: 50, or the nucleotide sequence from positions 192 to 2840 of SEQ ID NO: 52, and each of which encodes a protein that is functionally equivalent (a preferred function is the selective expression in Purkinje progenitor cells) to a protein comprising the amino acid sequence of SEQ ID NO: 47, 49, 51, or 53. The polypeptides also include isoforms, alternative isoforms, and allelic mutants of E-cadherin.

Such polynucleotides can be obtained from cDNA libraries and genomic libraries of animals such as humans, mice, rats, rabbits, hamsters, chickens, pigs, cows, goats, sheep, monkeys, and dogs by known hybridization methods such as colony hybridization, plaque hybridization, and Southern blotting using as a probe a polynucleotide comprising the nucleotide sequence from positions 1 to 4828 of SEQ ID NO: 46, the nucleotide sequence from positions 1 to 4413 of SEQ ID NO: 48, the nucleotide sequence from positions 1 to 4396 of SEQ ID NO: 50, or the nucleotide sequence from positions 1 to 4877 of SEQ ID NO: 52; preferably the nucleotide sequence from positions 125 to 2773 of SEQ ID NO: 46, the nucleotide sequence from positions 128 to 2782 of SEQ ID NO: 48, the nucleotide sequence from positions 127 to 2787 of SEQ ID NO: 50, or the nucleotide sequence from positions 192 to 2840 of SEQ ID NO: 52. Regarding methods for constructing cDNA libraries, one can refer to “Molecular Cloning, A Laboratory Manual 2^{nd} ed.”(Cold Spring Harbor Press (1989)). It is also possible to use cDNA libraries and genomic libraries available on the market.

Meanwhile, the phrase “functionally equivalent” means that a target protein has the same biological property as a 65B13 protein (for example, the amino acid sequence of SEQ ID NO: 2, 4, 20, 22, 24, 26, 28, 30, 32, 34, or 36) or an E-cadherin protein (for example, the amino acid sequence of SEQ ID NO: 47, 49, 51, or 53). The biological property of a 65B13 protein (for example, the amino acid sequence of SEQ ID NO: 2, 4, 20, 22, 24, 26, 28, 30, 32, 34, or 36) includes, for example, the olfactory nerve network pattern. Furthermore, the selective expression in Purkinje progenitor cells can also be regarded as a function (biological property). The biological property (function) of an E-cadherin (for example, the amino acid sequence of SEQ ID NO: 47, 49, 51, or 53) includes, for example, selective expression in Purkinje progenitor cells.

Accordingly, whether a target protein has the equivalent biological property as the 65B13 protein (amino acid sequence of SEQ ID NO: 2, 4, 20, 22, 24, 26, 28, 30, 32, 34, or 36) identified by the present inventors can be assessed by those skilled in the art, for example, by testing for the olfactory nerve network pattern or selective expression in Purkinje progenitor cells.

Similarly, whether a target protein has the equivalent biological property as the E-cadherin protein (amino acid sequence of SEQ ID NO: 47, 49, 51, or 53) can be assessed by those skilled in the art, for example, by testing for selective expression in Purkinje progenitor cells.

More specifically, in constructing a cDNA library, total RNA is first prepared from cells, organs, tissues, or such that express a polynucleotide of the present invention, by known techniques such as guanidine ultracentrifugation (Chirwin et al. Biochemistry 1979, 18:5294-5299) or AGPC (Chomczynski and Sacchi Anal. Biochem. 1987, 162:156-159), followed by mRNA purification using an mRNA purification kit (Pharmacia), etc. A kit for direct mRNA preparation, such as the QuickPrep mRNA Purification Kit (Pharmacia), may also be used. Next, cDNA is synthesized from the resulting mRNA using reverse transcriptase. cDNA synthesis kits such as the AMV Reverse Transcriptase First-strand cDNA Synthesis Kit (Seikagaku Corporation) are also available commercially. Other methods that use the 5′-RACE method to synthesize and amplify cDNA by PCR may also be used (Frohman et al. Proc. Natl. Acad. Sci. USA 1988, 85:8998-9002; Belyaysky et al. Nucleic Acids Res. 1989, 17:2919-32). In addition, in order to construct cDNA libraries containing a high percentage of full-length clones, known techniques such as the oligo-capping method (Maruyama and Sugano. Gene 1994, 138:171-4; Suzuki. Gene 1997, 200:149-56) can also be employed. The cDNA obtained in this manner is then incorporated into a suitable vector.

Examples of hybridization conditions in the present invention include “2×SSC, 0.1% SDS, 50° C.”, “2×SSC, 0.1% SDS, 42° C.”, and “1×SSC, 0.1% SDS, 37° C.”. Examples of conditions of higher stringency include “2×SSC, 0.1% SDS, 65° C.”, “0.5×SSC, 0.1% SDS, 42° C.”, and “0.2×SSC, 0.1% SDS, 65° C.”. More specifically, a method that uses the Rapid-hyb buffer (Amersham Life Science) can be carried out by performing pre-hybridization at 68° C. for 30 minutes or more, adding a probe to allow hybrid formation at 68° C. for one hour or more, washing three times in 2×SSC, 0.1% SDS at room temperature for 20 minutes each, washing three times in 1×SSC, 0.1% SDS at 37° C. for 20 minutes each, and finally washing twice in 1×SSC, 0.1% SDS at 50° C. for 20 minutes each. This can also be carried out using, for example, the Expresshyb Hybridization Solution (CLONTECH), by performing pre-hybridization at 55° C. for 30 minutes or more, adding a labeled probe and incubating at 37° C. to 55° C. for one hour or more, washing three times in 2×SSC, 0.1% SDS at room temperature for 20 minutes each, and washing once at 37° C. for 20 minutes with 1×SSC, 0.1% SDS. Here, conditions of higher stringency can be achieved by increasing the temperature for pre-hybridization, hybridization, or second wash. For example, the pre-hybridization and hybridization temperature can be raised to 60° C., and to 68° C. for higher stringency. In addition to conditions such as salt concentration of the buffer and temperature, a person with ordinary skill in the art can also integrate other conditions such as probe concentration, probe length, and reaction time, to obtain isoforms of 65B13 and E-cadherin of the present invention, allelic mutants, and corresponding genes derived from other organisms.

References such as “Molecular Cloning, A Laboratory Manual 2^{nd} ed.” (Cold Spring Harbor Press (1989), Sections 9.47-9.58), “Current Protocols in Molecular Biology” (John Wiley & Sons (1987-1997), Sections 6.3-6.4), “DNA Cloning 1: Core Techniques, A Practical Approach 2^{nd} ed.” (Oxford University (1995), Section 2.10 for conditions in particular) can be referred to for detailed information on hybridization procedures.

Examples of hybridizing polynucleotides include polynucleotides containing a nucleotide sequence that has at least 50% or more, preferably 70%, more preferably 80%, and even more preferably 90% (for example, 95% or more, or 99%) identity with a nucleotide sequence comprising the nucleotide sequence from positions 178 to 2280 of SEQ ID NO: 1, the nucleotide sequence from positions 127 to 2079 of SEQ ID NO: 3, the nucleotide sequence from positions 668 to 2770 of SEQ ID NO: 19, the nucleotide sequence from positions 130 to 2232 of SEQ ID NO: 21, the nucleotide sequence from positions 199 to 2100 of SEQ ID NO: 23, the nucleotide sequence from positions 199 to 2325 of SEQ ID NO: 25, the nucleotide sequence from positions 15 to 2325 of SEQ ID NO: 27, the nucleotide sequence from positions 15 to 1916 of SEQ ID NO: 29, the nucleotide sequence from positions 199 to 1950 of SEQ ID NO: 31, the nucleotide sequence from positions 15 to 1766 of SEQ ID NO: 33, or the nucleotide sequence from positions 196 to 2262 of SEQ ID NO: 35; or the nucleotide sequence from positions 125 to 2773 of SEQ ID NO: 46, the nucleotide sequence from positions 128 to 2782 of SEQ ID NO: 48, the nucleotide sequence from positions 127 to 2787 of SEQ ID NO: 50, or the nucleotide sequence from positions 192 to 2840 of SEQ ID NO: 52.

Such identities can be determined by the BLAST algorithm (Altschul. Proc. Natl. Acad. Sci. USA 1990, 87:2264-8; Karlin and Altschul. Proc. Natl. Acad. Sci. USA 1993, 90:5873-7). Examples of programs that have been developed based on this algorithm include the BLASTX program for determining the identity of amino acid sequences, and the BLASTN program for nucleotide sequences (Altschul et al. J. Mol. Biol. 1990, 215:403-10). These programs can be used for the sequences of the present invention. One can refer to, for example, http://www.ncbi.nlm.nih.gov for a specific example of analysis methods.

The isoforms or allelic mutants of 65B13 and E-cadherin, and other genes with a 65B13- or E-cadherin-like structure and function can be obtained from cDNA libraries and genomic libraries of animals such as humans, mice, rats, rabbits, hamsters, chickens, pigs, cows, goats, sheep, monkeys, and dogs by designing primers based on the nucleotide sequence from positions 178 to 2280 of SEQ ID NO: 1, the nucleotide sequence from positions 127 to 2079 of SEQ ID NO: 3, the nucleotide sequence from positions 668 to 2770 of SEQ ID NO: 19, the nucleotide sequence from positions 130 to 2232 of SEQ ID NO: 21, the nucleotide sequence from positions 199 to 2100 of SEQ ID NO: 23, the nucleotide sequence from positions 199 to 2325 of SEQ ID NO: 25, the nucleotide sequence from positions 15 to 2325 of SEQ ID NO: 27, the nucleotide sequence from positions 15 to 1916 of SEQ ID NO: 29, the nucleotide sequence from positions 199 to 1950 of SEQ ID NO: 31, the nucleotide sequence from positions 15 to 1766 of SEQ ID NO: 33, or the nucleotide sequence from positions 196 to 2262 of SEQ ID NO: 35; or the nucleotide sequence from positions 125 to 2773 of SEQ ID NO: 46, the nucleotide sequence from positions 128 to 2782 of SEQ ID NO: 48, the nucleotide sequence from positions 127 to 2787 of SEQ ID NO: 50, or the nucleotide sequence from positions 192 to 2840 of SEQ ID NO: 52, using gene amplification technology (PCR) (Current Protocols in Molecular Biology (John Wiley & Sons (1987), Sections 6.1-6.4)).

The polynucleotide sequences of the present invention can be confirmed by using conventional sequence determination methods. For example, the dideoxynucleotide chain termination method (Sanger et al. Proc. Natl. Acad. Sci. USA 1977, 74:5463) can be used. In addition, sequences can also be analyzed using a suitable DNA sequencer.

Specifically, in a preferred embodiment, the methods of the present invention include methods for detecting or selecting Purkinje progenitor cells, which comprise the steps of:

(A) detecting the expression of a polynucleotide capable of hybridizing to a polynucleotide selected from (i), (ii), (iii), and (iv) above or a complementary sequence thereof; and
(B) detecting the expression of a polynucleotide encoding an E-cadherin protein.

The Purkinje progenitor cells of the present invention preferably include, but are not limited to, Purkinje progenitor cells of the cerebellum.

The length of a polynucleotide of the present invention is not particularly limited, as long as it allows for detection or selection of Purkinje progenitor cells. The polynucleotides of the present invention also include the so-called “oligonucleotides”. In general, the polynucleotides of the present invention comprise at least ten consecutive nucleotides in the nucleotide sequence of the present invention or a complementary sequence thereof, and preferably comprise at least 15 consecutive nucleotides.

The “step of detecting the expression of a polynucleotide” in the above-described methods of the present invention preferably comprises the steps of:

(a) contacting a test cell sample with a polynucleotide that can hybridize to a polynucleotide selected by the above-described methods of the present invention or to a complementary sequence thereof, or with a probe comprising the polynucleotide; and
(b) detecting reactivity.

Alternatively, in a preferred embodiment of the methods of the present invention, first, the test cell sample is contacted with a polynucleotide of the present invention or with a probe comprising the polynucleotide. For example, it is possible to contact such a probe with mRNA prepared from the test cell sample or with a complementary DNA (cDNA) transcribed from the mRNA.

The absence and presence of reactivity is then determined in these methods. Herein, the presence of reactivity generally means that the contacted polynucleotide hybridizes (reacts) with the target sequence.

The steps of the above-described methods of the present invention may include, for example, the steps of:

(a-1) conducting gene amplification using as a template a polynucleotide derived from the test cell sample, and primers comprising polynucleotides that are capable of hybridizing to a polynucleotide of the present invention or a complementary sequence thereof, or a set of primers comprising polynucleotides that are capable of hybridizing to a polynucleotide selected in the present invention or a complementary sequence thereof; and
(b-1) detecting the resulting amplification products.

The “gene amplification method” in the above step includes known methods, for example, PCR. Furthermore, the amplification products generated by the amplification method can also be detected by known methods.

Meanwhile, for example, mRNA prepared from a test cell sample or complementary DNA (cDNA) transcribed from the mRNA may be used as a template in step (a-1) described above.

The detection step may be followed by the step of isolating Purkinje progenitor cells from the detected sample.

Since the protein encoded by the 65B13 gene of the present invention is a membrane protein, viable GABA-producing neuron progenitor cells can be isolated (separated) by using the protein as an indicator.

Similarly, since the protein encoded by the E-cadherin gene is a membrane protein, viable Purkinje progenitor cells can be isolated (separated) by using the protein as an indicator.

In addition, the methods of the present invention may comprise, in addition to the above-described step, the step of detecting or selecting Purkinje progenitor cells using the expression of a gene selected from the group consisting of Lim1/2 and Cor12 genes as an indicator.

Furthermore, the present invention also provides nucleotide chains complementary to a polynucleotide selected from (A) and (B) above for detecting or selecting Purkinje progenitor cells of the present invention, which comprise at least 15 consecutive nucleotides. Such polynucleotides comprising a nucleotide sequence that contains at least 15 consecutive nucleotides are useful as probes for detecting the generation of Purkinje progenitor cells or as primers for detecting Purkinje progenitor cells.

The nucleotide chain normally consists of 15 to 100, and preferably 15 to 35 nucleotides and the polynucleotide is appropriately labeled with a radioisotope, non-radioactive compound, or the like when used as a probe. The nucleotide chain preferably consists of at least 15 and preferably 30 nucleotides when used as a primer. A primer can be designed to have a restriction enzyme recognition sequence, a tag or such, added to the 5′-end side thereof, and at the 3′ end, a sequence complementary to a target sequence. A nucleotide chain of the present invention can hybridize with a polynucleotide of the present invention. Moreover, mutations of a polynucleotide of the present invention within cells can be detected using these probes or primers. In some cases, such mutations may cause abnormalities in the activity or expression of the polypeptides of the present invention; therefore, nucleotide chains of the present invention are thought to be useful for disease diagnosis, etc.

Here, a “complementary sequence” refers to not only cases where at least 15 consecutive nucleotides of the nucleotide sequence completely pair with the template, but also includes those that have at least 70%, preferably 80%, more preferably 90%, and even more preferably 95% or more (for example, 97% or 99%) of the consecutive nucleotides paired with the template. Pair formation refers to the formation of a chain, in which T (U in the case of an RNA) corresponds to A, A corresponds to T or U, G corresponds to C, and C corresponds to G in the nucleotide sequence of the template polynucleotide. Identities can be determined by methods similar to that used in the aforementioned polynucleotide hybridization.

The present invention also provides primer sets comprising two or more polynucleotides selected from (A) and (B) above for detecting or selecting Purkinje progenitor cells of the present invention.

Furthermore, the present invention provides kits for detecting or selecting Purkinje progenitor cells. The kits of the present invention may comprise, for example, probes, primers, or primer sets that enable detection of the expression of a polynucleotide that can hybridize to a polynucleotide selected from (A) and (B) described above, or to a complementary sequence thereof. The kits may also comprise appropriate buffers, etc. Furthermore, the packages may contain instruction manuals containing a description of how to use the kits.

In an alternative embodiment, the kits of the present invention for detecting or selecting Purkinje progenitor cells include kits comprising antibodies that bind to either of the proteins of (A) and (B) below:

(A) a protein selected from (v), (vi), (vii), and (viii) above; and
(B) a protein selected from (v′), (vi′), (vii′), and (viii′) above.

In an alternative embodiment, the kits of the present invention include kits comprising a polynucleotide encoding a marker protein under the control of a promoter that controls (induces) expression of an mRNA to be translated into a protein selected from (A) and (B) below:

(A) a protein selected from (v), (vi), (vii), and (viii) above; and
(B) a protein selected from (v′), (vi′), (vii′), and (viii′) above.

The kits of the present invention may further comprise polynucleotides that hybridize to transcripts of one or more genes selected from the group consisting of the Lim1/2 and Cor12 genes.

The kits may further contain in combination antibodies that bind to proteins encoded by one or more genes selected from the group consisting of Lim1/2 and Cor12 genes.

The kits of the present invention may further comprise Purkinje progenitor cells as a positive control.

The present invention also provides methods for preparing a Purkinje progenitor cell population. Such methods include, for example, methods comprising the steps of:

(VII) preparing a cell population that potentially contains Purkinje progenitor cells;
(VIII) detecting Purkinje progenitor cells using a method of the present invention for detecting or selecting Purkinje progenitor cells; and
(IX) expanding cells detected or selected in step (VIII).

The Purkinje progenitor cells obtained by the production methods described above are used, for example, in treating cerebellar degeneration.

The Purkinje progenitor cells obtained by the production methods described above are also included in the present invention. It is preferable that cells produced by the above-described methods of the present invention are viable cells.

Since cells obtained in the present invention are Purkinje progenitor cells, they are preferable in transplant therapy for degenerative diseases and such in terms of their safety, survival rate, and network formation ability, compared to mixed cell populations or Purkinje progenitor cells carrying an exogenous gene. Moreover, since cells (or cell populations) of the present invention obtained according to the methods are progenitor cells, they can be differentiated into a suitable stage by selecting in vitro conditions such as media, and are preferable materials for various types of neural transplant therapy. When Purkinje progenitor cells obtained using the methods of the present invention are used in transplants, preferably 1×10³ to 1×10⁶ Purkinje cells, and more preferably 5×10⁴ to 6×10⁴ Purkinje cells are transplanted. The primary method is stereotaxic surgery in which a cell suspension is transplanted into the brain. In addition, cells may also be transplanted by microsurgery. See, Backlund et al. (J. Neurosurg. 1985, 62:169-73), Lindvall et al. (Ann. Neurol. 1987, 22:457-68), or Madrazo et al. (New Engl. J. Med. 1987, 316:831-4) for methods of transplanting Purkinje tissues.

Moreover, the cells of the present invention can also be used to isolate genes specific to Purkinje progenitor cells, and genes specific to each stage of the maturation from progenitor cells into Purkinje cells. They can also be used for searching therapeutic targets for degenerative diseases, elucidating the maturation process of Purkinje cells, and in screenings using maturation as an indicator.

The present invention also provides reagents for detecting or selecting Purkinje progenitor cells.

In an embodiment, the reagents of the present invention include, for example, reagents for detecting or selecting Purkinje progenitor cells, which comprise probes, primers, or primer sets that enable detection of the expression of a polynucleotide that can hybridize to a polynucleotide selected from (A) and (B) described above, or to a complementary sequence thereof.

In an alternative embodiment, such reagents of the present invention for detecting or selecting Purkinje progenitor cells include, for example, reagents for detecting or selecting Purkinje progenitor cells, which contain an antibody that binds to a protein selected from (A) and (B) below:

(A) a protein selected from (v), (vi), (vii), and (viii) above; and
(B) a protein selected from (v′), (vi′), (vii′), and (viii′) above.

In an alternative embodiment, such reagents of the present invention for detecting or selecting Purkinje progenitor cells include, for example, reagents for detecting or selecting Purkinje progenitor cells, which contain a polynucleotide to be used to link a marker protein-encoding polynucleotide under the control of a promoter that controls (induces) expression of an mRNA to be translated into a protein selected from (A) and (B) below:

(A) a protein selected from (v), (vi), (vii), and (viii) above; and
(B) a protein selected from (v′), (vi′), (vii′), and (viii′) above.

Herein, the “cell type identification” means not only when target cells are identified to be of a specific cell type, but also when target cells are judged not to be of a specific cell type. For example, when the 65B13 gene and E-cadherin gene are substantially expressed in target cerebellar cells, the cells can be identified to be “possibly Purkinje progenitor cells”.

The present invention also provides reagents for detecting or selecting cerebellar cells. For the reagents, the above-described reagents for detecting or selecting Purkinje progenitor cells may be appropriately combined with other known markers. Such reagents enable thorough cell type identification.

Thus, in a preferred embodiment, the present invention provides reagents for detecting or selecting cerebellar cells, which comprise a combination of the above-described reagents for detecting or selecting Purkinje progenitor cells and polynucleotides that hybridize to the transcripts of one or more genes selected from the group consisting of Lim1/2 and Cor12 genes.

The sequences of the above-described marker genes are known as listed below.

The nucleotide sequence of mouse Lim1 is shown in SEQ ID NO: 5 and the amino acid sequence is shown in SEQ ID NO: 6; the nucleotide sequence of human Lim 1 is shown in SEQ ID NO: 7 and the amino acid sequence is shown in SEQ ID NO: 8.

The nucleotide sequence of mouse Lim2 is shown in SEQ ID NO: 9 and the amino acid sequence is shown in SEQ ID NO: 10; the nucleotide sequence of human Lim2 is shown in SEQ ID NO: 11 and the amino acid sequence is shown in SEQ ID NO: 12; the nucleotide sequence of rat Lim2 is shown in SEQ ID NO: 13 and the amino acid sequence is shown in SEQ ID NO: 14.

The nucleotide sequence of mouse Cor12 is shown in SEQ ID NO: 15 and the amino acid sequence is shown in SEQ ID NO: 16; the nucleotide sequence of human Cor12 is shown in SEQ ID NO: 17 and the amino acid sequence is shown in SEQ ID NO: 18.

The above-described reagents for detecting or selecting Purkinje progenitor cells may further comprise in combination antibodies that bind to proteins encoded by one or more genes selected from the group consisting of Lim1/2 and Cor12 genes.

Cerebellar cells that can be identified using the above-described reagents include Purkinje cells.

The above-described reagents may further comprise cerebellar cells.

The present invention also provides polynucleotides for detecting or selecting Purkinje progenitor cells for use in regeneration medicine to treat cerebellar degeneration.

In a preferred embodiment, such polynucleotides of the present invention include polynucleotides capable of hybridizing to a polynucleotide selected from (A) and (B) below or a complementary sequence thereof:

(A) a polynucleotide selected from (i), (ii), (iii), and (iv) above; and
(B) a polynucleotide selected from (i′), (ii′), (iii′), and (iv′) above.

The present invention also provides antibodies that bind to a translation product of the 65B13 gene or E-cadherin gene for use in regeneration medicine for cerebellar degeneration. In a preferred embodiment, such antibodies of the present invention include, for example, antibodies for detecting or selecting Purkinje progenitor cells for use in regeneration medicine for cerebellar degeneration, which bind to a protein selected from (A) and (B) below:

### (A)

(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and

### (B)

(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53.

In a preferred embodiment, the antibodies of the present invention include antibodies that bind to a polypeptide selected from (A) and (B) below:

(A) an extracellular domain polypeptide of GABA-producing neuron progenitor cells; and
(B) an extracellular domain polypeptide of Purkinje progenitor cells.

The extracellular domains of polypeptides used in the present invention can be searched using the program, PSORT (http://psort.ims.u-tokyo.ac.jp/), etc. Specifically, the extracellular domains of a polypeptide encoding the 65B13 protein obtained using the PSORT program are: the amino acid sequences from positions 21 to 510 of SEQ ID NO: 2, 4, 24, 26, 28, or 30; positions 20 to 513 of SEQ ID NO: 20 or 22; positions 21 to 460 of SEQ ID NO: 32 or 34; and positions 21 to 490 of SEQ ID NO: 36. Meanwhile, the extracellular domains of a polypeptide encoding the E-cadherin protein are: the amino acid sequences from positions 23 to 708 of SEQ ID NO: 47; positions 23 to 710 of SEQ ID NO: 49; positions 23 to 712 of SEQ ID NO: 51; and positions 23 to 708 of SEQ ID NO: 53.

Antibodies of the present invention also include polyclonal antibodies, monoclonal antibodies, chimeric antibodies, single-chain antibodies (scFV) (Huston et al. Proc. Natl. Acad. Sci. USA 1988, 85:5879-83; The Pharmacology of Monoclonal Antibody, vol. 113, Rosenburg and Moore ed., Springer Verlag (1994) pp. 269-315), humanized antibodies, multispecific antibodies (LeDoussal et al. Int. J. Cancer Suppl. 1992, 7:58-62; Paulus. Behring Inst. Mitt. 1985, 78:118-32; Millstein and Cuello. Nature 1983, 305:537-9; Zimmermann Rev. Physiol. Biochem. Pharmacol. 1986, 105:176-260; Van Dijk et al. Int. J. Cancer 1989, 43:944-9), and antibody fragments such as Fab, Fab′, F(ab′)2, Fc, and Fv. Moreover, an antibody of the present invention may also be modified by PEG and such, as necessary. An antibody of the present invention may also be produced in the form of a fusion protein with β-galactosidase, maltose-binding protein, GST, green fluorescent protein (GFP), or such, to allow detection without the use of a secondary antibody. In addition, an antibody may be modified by labeling with biotin or such to allow recovery using avidin, streptoavidin, etc.

An antibody of the present invention can be produced using a polypeptide of the present invention, a fragment thereof, or a cell in which a polypeptide or polypeptide fragment of the present invention is expressed, as a sensitized antigen. In addition, a short polypeptide of the present invention or a fragment thereof may also be used as an immunogen by coupling to a carrier such as bovine serum albumin, Keyhole Limpet Hemocyanin, and ovalbumin. In addition, a polypeptide of the present invention or a fragment thereof may be used in combination with a known adjuvant such as aluminum adjuvant, Freund's complete (or incomplete) adjuvant, or pertussis adjuvant, to enhance the immune response to an antigen.

Polyclonal antibodies can be obtained from, for example, the serum of an immunized animal after immunizing a mammal with a polypeptide of the present invention or a fragment thereof, together with an adjuvant as necessary. Although there are no particular limitations on the mammals used, typical examples include rodents, lagomorphs, and primates. Specific examples include rodents such as mice, rats, and hamsters; lagomorphs such as rabbits; and primates such as monkeys including cynomolgus monkeys, rhesus monkeys, baboons, and chimpanzees. Animal immunization is carried out by suitably diluting and suspending a sensitized antigen in phosphate-buffered saline (PBS) or physiological saline, mixing with an adjuvant as necessary until emulsified, and injecting into an animal intraperitoneally or subcutaneously. The sensitized antigen mixed with Freund's incomplete adjuvant is preferably administered several times, every 4 to 21 days. Antibody production can be confirmed by measuring the level of an antibody of interest in the serum using conventional methods. Finally, the serum itself may be used as a polyclonal antibody, or it may be further purified. See, for example, “Current Protocols in Molecular Biology” (John Wiley & Sons (1987), Sections 11.12-11.13), for specific methods.

A monoclonal antibody can be produced by removing the spleen from an animal immunized in the manner described above, separating immunocytes from the spleen, and fusing with a suitable myeloma cell using polyethylene glycol (PEG) or such to establish hybridomas. Cell fusion can be carried out according to the Milstein method (Galfre and Milstein. Methods Enzymol. 1981, 73:3-46). Here, suitable myeloma cells are exemplified particularly by cells that allow chemical selection of fused cells. When using such myeloma cells, fused hybridomas are selected by culturing in a culture medium (HAT culture medium) that contains hypoxanthine, aminopterin, and thymidine, which destroy cells other than the fused cells. Next, a clone that produces an antibody against a polypeptide of the present invention or a fragment thereof is selected from the established hybridomas. Subsequently, the selected clone is introduced into the abdominal cavity of a mouse or such, and ascite is collected to obtain a monoclonal antibody. See, in addition, “Current Protocols in Molecular Biology” (John Wiley & Sons (1987), Sections 11.4-11.11) for information on specific methods.

Hybridomas can also be obtained by first sensitizing human lymphocytes that have been infected by EB virus with an immunogen in vitro, and fusing the sensitized lymphocytes with human myeloma cells (such as U266) to obtain hybridomas that produce human antibodies (JP-A (Kokai) S63-17688). In addition, human antibodies can also be obtained by using antibody-producing cells generated by sensitizing a transgenic animal with a human antibody gene repertoire (WO 92/03918; WO 93/02227; WO 94/02602; WO 94/25585; WO 96/33735; WO 96/34096; Mendez et al. Nat. Genet. 1997, 15:146-156, etc.). Methods that do not use hybridomas can be exemplified by a method in which a cancer gene is introduced to immortalize immunocytes such as antibody-producing lymphocytes.

In addition, antibodies can also be produced by genetic recombination techniques (see Borrebaeck and Larrick (1990) Therapeutic Monoclonal Antibodies, MacMillan Publishers Ltd., UK). First, a gene that encodes an antibody is cloned from hybridomas or antibody-producing cells (such as sensitized lymphocytes). The resulting gene is then inserted into a suitable vector, the vector is introduced into a host, and the host is then cultured to produce the antibody. This type of recombinant antibody is also included in the antibodies of the present invention. Typical examples of recombinant antibodies include chimeric antibodies comprising a non-human antibody-derived variable region and a human antibody-derived constant region, and humanized antibodies comprising a non-human-derived antibody complementarity determining region (CDR), human antibody-derived framework region (FR), and human antibody constant region (Jones et al. Nature 1986, 321:522-5; Reichmann et al. Nature 1988, 332: 323-9; Presta. Curr. Op. Struct. Biol. 1992, 2:593-6; Methods Enzymol. 1991, 203:99-121).

Antibody fragments of the present invention can be produced by treating the aforementioned polyclonal or monoclonal antibodies with enzymes such as papain or pepsin. Alternatively, an antibody fragment can be produced by genetic engineering techniques using a gene that encodes an antibody fragment (see Co et al. J. Immunol. 1994, 152:2968-76; Better and Horwitz. Methods Enzymol. 1989, 178:476-96; Pluckthun and Skerra. Methods Enzymol. 1989, 178:497-515; Lamoyi. Methods Enzymol. 1986, 121:652-63; Rousseaux et al. 1986, 121:663-9; Bird and Walker. Trends Biotechnol. 1991, 9:132-7).

The multispecific antibodies of the present invention include bispecific antibodies (BsAb), diabodies (Db), etc. Multispecific antibodies can be produced by methods such as (1) chemically coupling antibodies having different specificities with different types of bifunctional linkers (Paulus Behring Inst. Mill. 1985, 78:118-32), (2) fusing hybridomas that secrete different monoclonal antibodies (Millstein and Cuello. Nature 1983, 305:537-9), or (3) transfecting eukaryotic cell expression systems, such as mouse myeloma cells, with a light chain gene and a heavy chain gene of different monoclonal antibodies (four types of DNA), followed by the isolation of a bispecific monovalent portion (Zimmermann. Rev. Physio. Biochem. Pharmacol. 1986, 105:176-260; Van Dijk et al. Int. J. Cancer 1989, 43:944-9). On the other hand, diabodies are dimer antibody fragments comprising two bivalent polypeptide chains that can be constructed by gene fusion. They can be produced using known methods (see Holliger et al. Proc. Natl. Acad. Sci. USA 1993, 90:6444-8; EP404097; WO 93/11161).

Recovery and purification of antibodies and antibody fragments can be carried out using Protein A and Protein G, or according to known protein purification techniques (Antibodies: A Laboratory Manual, Ed. Harlow and David Lane, Cold Spring Harbor Laboratory (1988)). For example, when using Protein A to purify an antibody of the present invention, known Protein A columns such as Hyper D, POROS, or Sepharose F.F. (Pharmacia) can be used. The concentration of the resulting antibody can be determined by measuring the absorbance or by enzyme linked immunoadsorbent assay (ELISA).

Antigen-binding activity of an antibody can be determined by absorbance measurement, or by using fluorescent antibody methods, enzyme immunoassay (ETA) methods, radioimmunoassay (RIA) methods, or ELISA. When ELISA is used, an antibody of the present invention is first immobilized onto a support such as a plate. A polypeptide of the present invention is added, and then a sample containing the antibody of interest is added. Here, samples containing an antibody of interest include, for example, culture supernatants of antibody-producing cells, purified antibodies, etc. Next, a secondary antibody that recognizes an antibody of the present invention is added, followed by the incubation of the plate. Subsequently, the plate is washed and the label attached to the secondary antibody is detected. Namely, if a secondary antibody is labeled with alkaline phosphatase, the antigen binding activity can be determined by adding an enzyme substrate such as p-nitrophenyl phosphate, and measuring the absorbance. In addition, a commercially available system such as BIAcore (Pharmacia) can also be used to evaluate antibody activities.

The antibodies of the present invention can recognize or detect a polypeptide of the present invention or a fragment thereof. Furthermore, since the antibodies recognize a polypeptide of the present invention or a fragment thereof, they can recognize or detect cells or the like expressing the polypeptide or a fragment thereof. In addition, the antibodies can be used to purify a polypeptide of the present invention or a fragment thereof. The antibodies can also be used to purify cells or the like expressing the polypeptide of the present invention or a fragment thereof.

The antibodies of the present invention preferably bind to a polypeptide comprising the entire or at least six consecutive amino acid residues of the amino acid sequence of (A′) and (B′) described below, and more preferably bind to a polypeptide comprising at least six consecutive amino acid residues of the above amino acid sequence.

(A′) the amino acid sequences from positions 21 to 510 of SEQ ID NO: 2, 4, 24, 26, 28, or 30; positions 20 to 513 of SEQ ID NO: 20 or 22; positions 21 to 460 of SEQ ID NO: 32 or 34; and positions 21 to 490 of SEQ ID NO: 36.
(B′) the amino acid sequences from positions 23 to 708 of SEQ ID NO: 47; positions 23 to 710 of SEQ ID NO: 49; positions 23 to 712 of SEQ ID NO: 51; and positions 23 to 708 of SEQ ID NO: 53.

In addition, it is necessary that a support used in immobilizing an antibody or a polypeptide of the present invention is safe to cells. Examples of such a support include synthetic or naturally occurring organic polymer compounds, inorganic materials such as glass beads, silica gel, alumina, and activated charcoal, and those that have their surfaces coated with a polysaccharide or synthetic polymer. There are no particular limitations on the form of the support, examples of which include films, fibers, granules, hollow fibers, non-woven fabric, porous supports, or honeycombed supports, and the contact surface area can be controlled by changing its thickness, surface area, width, length, shape, and size in various ways.

Marker proteins for Purkinje progenitor cells other than the proteins selected by the methods of the present invention include, for example, proteins encoded by genes selected from the group consisting of the Lim1/2 and Cor12 genes.

In the methods of the present invention, transcripts of the 65B13 gene and E-cadherin gene can be detected by contacting a polynucleotide of the present invention with nucleic acid extract derived from a cell sample, and detecting a nucleic acid that hybridizes to the polynucleotide in the nucleic acid extract.

The polynucleotide probe is preferably labeled with radioisotope or non-radioactive compound to detect transcripts of the 65B13 gene and E-cadherin gene. The radioisotopes to be used as a label include, for example, ³⁵S and ³H. When a radiolabeled polynucleotide probe is used, RNA that binds to a marker can be detected by detecting silver particles by emulsion autoradiography. Meanwhile, for conventional non-radioisotopic compounds used to label polynucleotide probes, biotin and digoxigenin are known. The detection of biotin-labeled markers can be achieved, for example, using fluorescently labeled avidin or avidin labeled with an enzyme such as alkaline phosphatase or horseradish peroxidase. On the other hand, the detection of digoxigenin-labeled markers can be achieved by using fluorescently labeled anti-digoxigenin antibody or anti-digoxigenin antibody labeled with an enzyme such as alkaline phosphatase or horseradish peroxidase. When enzyme labeling is used, the detection can be made by allowing stable dye to deposit at marker positions by incubation with an enzyme substrate.

When polynucleotide primers are used for detection of transcripts of the 65B13 gene and E-cadherin gene, their transcripts can be detected by amplifying nucleic acid that hybridizes to the polynucleotide primers, for example, using techniques such as RT-PCR.

Detection of the translation products of the 65B13 gene and E-cadherin gene with the methods of the present invention can be made by contacting the antibody described above with a protein extract of cell samples and then detecting proteins bound to the antibody. As described above, methods for assaying the antigen binding activity of an antibody include absorbance measurement, fluorescent antibody method, enzyme immunoassay (EIA), radioimmunoassay (RIA), ELISA, etc.

In the context of the present invention, highly accurate identification can be achieved by detecting, in addition to transcripts or translation products of the 65B13 gene and E-cadherin gene, the transcripts or translation products of one or more genes selected from the group consisting of Lim1/2 and Cor12. Such methods are also included in the present invention.

Furthermore, the expression of the polynucleotides of 65B13 and E-cadherin of the present invention can be used as an indicator to screen for substances that are effective for induction of differentiation into Purkinje progenitor cells.

The present invention provides methods of screening for substances that are effective for induction of differentiation into Purkinje progenitor cells. Since compounds obtained through screening by the methods of the present invention have the activity of differentiating Purkinje progenitor cells, they are potential candidate compounds useful for treating diseases caused by defects in Purkinje cells. Target diseases (illnesses) of treatment using a compound obtained by the screening methods include, for example, cerebellar degeneration.

In a preferred embodiment, the above-described screening methods of the present invention comprise the steps of:

(I) contacting a test compound with cells that can differentiate into Purkinje progenitor cells; and
(II) detecting in Purkinje progenitor cells the expression of a polynucleotide that can hybridize to a polynucleotide sequence selected from (A) and (B) described above or to a complementary sequence thereof.

Here, the “test substance” may be any type of compound, examples of which include the expression products of gene libraries, synthetic low-molecular-weight compound libraries, synthetic peptide libraries, antibodies, substances released by bacteria, cell (microbial, plant, or animal) extracts, cell (microbial, plant, or animal) culture supernatants, purified or partially purified polypeptides, marine organisms, plant or animal extracts, soil, random phage peptide display libraries, etc.

Furthermore, since 65B13 and E-cadherin are expressed selectively in differentiated Purkinje progenitor cells, it can be used to screen for reagents that induce differentiation into Purkinje progenitor cells. Specifically, whether a test sample has the ability to induce differentiation into Purkinje progenitor cells can be assessed by inducing the differentiation into Purkinje progenitor cells from cells having the ability to differentiate into Purkinje progenitor cells in the presence of the test sample, and detecting the expression of E-cadherin in the differentiated cells.

Thus, the present invention provides methods of screening for candidate compounds as a reagent that induces differentiation into Purkinje progenitor cells, which comprise the steps of:

(f) inducing the differentiation into Purkinje progenitor cells from cells having the ability to differentiate into Purkinje progenitor cells in the presence of a test sample;
(g) detecting transcripts or translation products of the 65B13 gene and E-cadherin gene in the differentiated cells; and
(h) selecting a compound that increases the level of the transcripts or translation products as compared to those detected in the absence of the test sample.

In the above methods, the preferred “cells having the ability to differentiate into Purkinje progenitor cells” are cell samples containing cells that can be differentiated into Purkinje progenitor cells, such as multipotent ES cells.

The transcripts or translation products of the 65B13 gene and E-cadherin gene of the present invention can be detected using polynucleotides that hybridize to a transcript of the 65B13 gene or E-cadherin gene, or antibodies that bind to a translation product of the 65B13 gene or E-cadherin gene, as described above.

In the present invention, cell growth and differentiation can be detected by comparing the cell condition with that when the test substance is not contacted. Cell growth and differentiation can be assessed through morphological observation under a microscope, or detecting or quantifying substances produced upon cell differentiation.

Cell differentiation can be assessed by comparing the expression levels of the 65B13 gene and E-cadherin gene with those in the absence of a test sample. Specifically, when a test sample increases the level of transcript or translation product of the 65B13 gene or E-cadherin gene as compared to that in the absence of the test sample, the test sample is assessed to have the ability to differentiate nerve cells. “Increase” means that, for example, the level becomes twice, preferably five times, and more preferably ten or more times.

In a preferred embodiment of the present invention, the screening methods of the present invention further comprise the step of selecting compounds in which the expression of the polynucleotide is detected in step (II).

In another preferred embodiment of the present invention, the screening methods of the present invention also include, for example, methods comprising:

(IV) the step of contacting a test compound with cells having the ability to differentiate into Purkinje progenitor cells; and
(V) the steps comprising (A) and (B) below:
(A) detecting a protein selected from (v), (vi), (vii), and (viii) described above; and
(B) detecting a protein selected from (v′), (vi′), (vii′), and (viii′) described above.

In a preferred embodiment, the above-described methods further comprise step (VI) of selecting compounds in which the protein is detected in step (V).

The above methods may be based on the use of promoters (including modified promoters) (as a means) that control (induce) expression of an mRNA translated into the protein of (A) or (B) below:

(A) a protein selected from (v), (vi), (vii), and (viii) above; or
(B) a protein selected from (v′), (vi′), (vii′), and (viii′).

For example, it is possible to transfect cells with a vector carrying a construct in which a gene encoding a detectable marker such as green fluorescent protein (GFP) is linked to a promoter portion obtained by analyzing the expression regulatory regions. Alternatively, a marker gene can be knocked in at the gene locus. The expression of a marker gene specific to GABA-producing neuron progenitor cells comprising Purkinje cells is detected by (A) and the expression of a marker gene specific to Purkinje progenitor cells is detected by (B). Combined use of the two enables detection of marker genes specific to Purkinje progenitor cells and thus enables detection of the protein. The protein expression can be detected by methods (means) for detecting protein expression, and therefore the methods can also be used as methods (means) for detecting the protein-encoding gene.

In this case, “the gene encoding a marker is linked to a promoter portion” means that the gene encoding the marker is linked to the promoter portion in an expressible manner. The gene may be directly linked to the promoter, or the gene may be linked distantly to but still under the control of the promoter. Furthermore, the promoter portion obtained by analyzing the 65B13 expression regulatory region may be replaced with another promoter, as long as the promoter for 65B13 enables the expression of the 65B13 region. The promoter portion obtained by analyzing the E-cadherin expression region may also be replaced with another promoter, as long as the promoter for E-cadherin enables the expression of the E-cadherin region.

All prior-art documents cited in the specification have been incorporated herein by reference.

### EXAMPLES

Hereinbelow, the present invention is specifically described with reference to the Examples; however, it should not be construed as being limited thereto.

### Example 1

### Analysis of 65B13 Expression in Cerebellar Primordium and Isolation of 65B13-Positive Cells

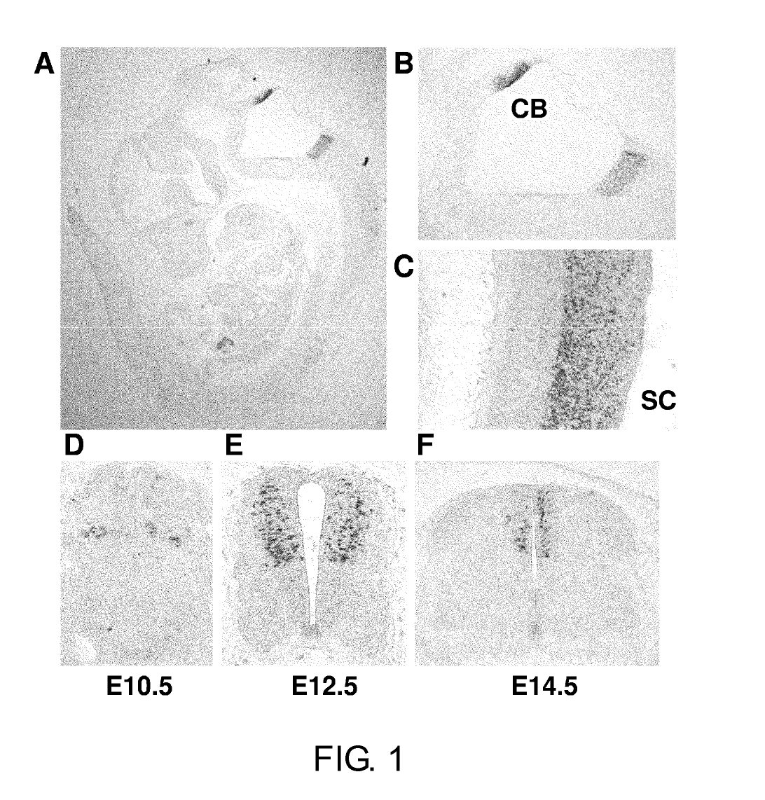

65B13 (Neph3) is selectively expressed in several areas of fetal brain (FIG. 1; WO 2004/038018; Minaki Y, Mizuhara E, Morimoto K, Nakatani T, Sakamoto Y, Inoue Y, Satoh K, Imai T, Takai Y, Ono Y. Migrating postmitotic neural precursor cells in the ventricular zone extend apical processes and form adherens junctions near the ventricle in the developing spinal cord. Neurosci Res. 2005, 52(3):250-62). In the midbrain, dopaminergic neuron progenitor cells have been demonstrated to express 65B13 (WO 2004/038018); however, the types of cells that express 65B13 remain unidentified in other areas. The cerebellum is constituted of glutamic acid-producing granule cells and GABA-producing neurons such as Purkinje cells, Golgi cells, stellate cells, and basket cells (Wang V Y, Zoghbi H Y. Genetic regulation of cerebellar development. Nat Rev Neurosci. 2001, 2(7):484-91). The granule cells are known to develop in the rhombic lip region at E12.5 to E14.5 (Wang V Y, Zoghbi H Y. Genetic regulation of cerebellar development. Nat Rev Neurosci. 2001, 2(7):484-91). By contrast, the development of GABA-producing neurons is still poorly understood. However, recent findings suggest that Purkinje cells are generated at E11.5 to E13.5 in the dorsal rhombomere 1 (cerebellar primordium area) (Chizhikov V V, Lindgren A G, Currle D S, Rose M F, Monuki E S, Millen K J. The roof plate regulates cerebellar cell-type specification and proliferation. Development. 2006, 133(15):2793-804). Although there is no detailed report on Golgi cells, they are thought to be generated at a late stage (E13.5 to E15.5) of development in the same region. The stellate cells and basket cells are thought to be generated from progenitor cells in the white matter after birth (Zhang L, Goldman J E. Generation of cerebellar interneurons from dividing progenitors in white matter. Neuron. 1996, 16(1):47-54).

To identify 65B13-expressing cells in the cerebellar primordium, the spatial expression pattern of 65B13 was compared with those of various markers. Mouse E12.5 embryos were collected and fixed in 4% PFA/PBS(−) at 4° C. for two hours. After replacing with 20% sucrose/PBS(−) at 4° C. overnight, the embryos were embedded in OCT. Sections of 12-μm thickness were prepared and placed onto slide glasses. Then, the sections were dried at room temperature for 30 minutes, and again wetted with PBS(−). Next, after 30 minutes of blocking (BlockAce) at room temperature, a primary antibody was reacted at room temperature for one hour. The reaction was followed by incubation at 4° C. overnight. The sections were washed three times with 0.1% Tween-20/PBS(−) at room temperature for 15 minutes each, and then incubated with a fluorescently labeled secondary antibody at room temperature for one hour. After washing in the same way, the sections were washed with PBS(−) at room temperature for ten minutes, and then mounted. The primary antibodies used were: 65B13 (see WO 2004/038018; Minaki Y, Mizuhara E, Morimoto K, Nakatani T, Sakamoto Y, Inoue Y, Satoh K, Imai T, Takai Y, Ono Y. Migrating postmitotic neural precursor cells in the ventricular zone extend apical processes and form adherens junctions near the ventricle in the developing spinal cord. Neurosci Res. 2005, 52(3):250-62), Pax2 (purchased from Zymed), and Cor12 (see WO 2006/082826).

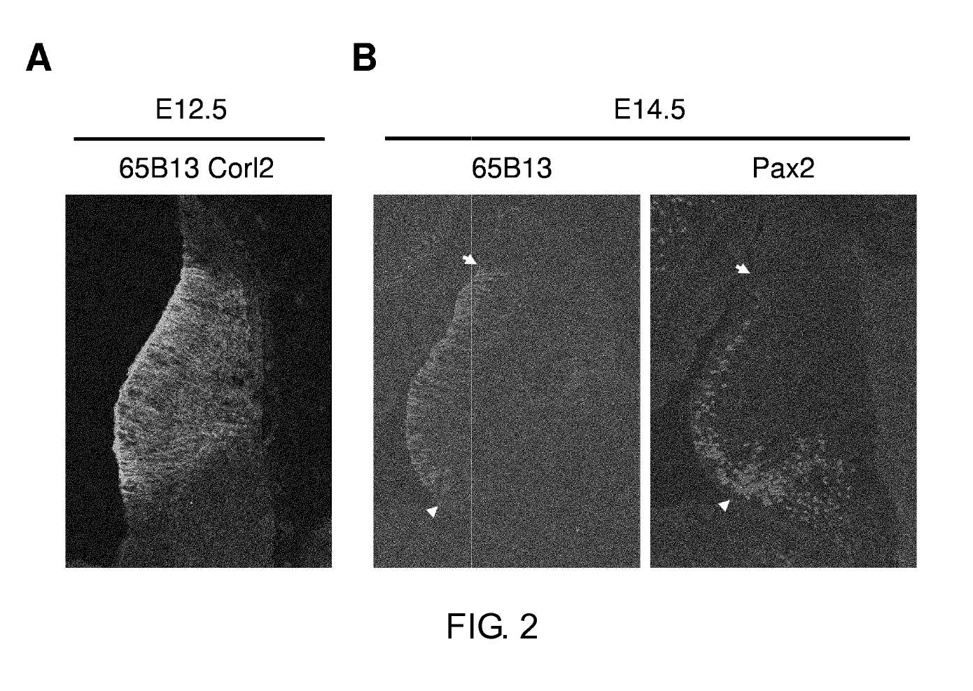

The result showed that in the cerebellar primordium of E12.5, 65B13 was selectively expressed in VZ where Cor12-positive Purkinje cells develop (FIG. 2A). In addition, neurons that are thought to be Pax2-positive Golgi cells revealed to start to emerge at E14.5 in the same 65B13-positive area (Maricich S M, Herrup K. Pax-2 expression defines a subset of GABAergic interneurons and their precursors in the developing murine cerebellum. J. Neurobiol. 1999, 41(2):281-94) (FIG. 2B).

Thus, experiments were carried out to isolate and culture 65B13-positive cells for the purpose of confirming that the 65B13-positive cells are progenitor cells of Purkinje and Golgi cells.

The cerebellar primordium areas at E12.5 and E14.5 were excised, and dispersed using Cell Dissociation Buffer (Invitrogen). Then, without fixation and permeability treatment, the cells were stained at 4° C. for 20 minutes using an anti-65B13 monoclonal antibody (100 times diluted purified antibody, 1% bovine fetal serum, 1 mM EDTA/SDIA differentiation medium (Kawasaki et al. Neuron 2000, 28(1):31-40)). After washing three times with 1 mM EDTA/PBS containing 1% bovine fetal serum at 4° C. for three minutes, the cells were stained with a PE-labeled anti-hamster IgG antibody (Jackson; 10 μg/ml, 1% bovine fetal serum, 1 mM EDTA/SDIA differentiation medium) at 4° C. for 30 minutes and washed in the same way as described above. After staining, 65B13-expressing cells were separated with a cell sorter. The isolated cells were placed onto slide glasses coated with poly-L-ornithine (Sigma, 0.002% in PBS), laminin (Invitrogen, 5 μg/ml in PBS), and fibronectin (Sigma, 5 μg/ml in PBS) and cultured at 37° C. for two days in SDIA differentiation medium supplemented with Knockout Serum Replacement (Gibco, 5%), N2 (Invitrogen, 1×), B27 (Invitrogen, 1×), ascorbic acid (Sigma, 200 μM), and BDNF (Invitrogen, 20 ng/ml). The cultured cells were fixed with 2% PFA/PBS at 4° C. for 20 minutes, and then washed twice with PBS at 4° C. for 10 minutes. Then, cell permeability treatment was performed using 0.3% Triton X-100/PBS at room temperature for 30 minutes, and the cells were blocked with 10% normal donkey serum/BlockAce at room temperature for 20 minutes. Next, the cells were incubated with a primary antibody (10% normal donkey serum, 2.5% BlockAce, 0.1% Triton X-100/PBS) at room temperature for one hour and then at 4° C. overnight. On the next day, after washing three times with 0.1% Triton X-100/PBS at room temperature for ten minutes, the cells were incubated with a fluorescently labeled secondary antibodies (all from Jackson, 10 μg/ml, 10% normal donkey serum, 2.5% BlockAce, 0.1% Triton X-100/PBS) at room temperature for 30 minutes. After washing in the same way as described above, the cells were washed with PBS at room temperature for five minutes, mounted, and observed. An anti-HuC/D antibody was purchased from Molecular Probe.

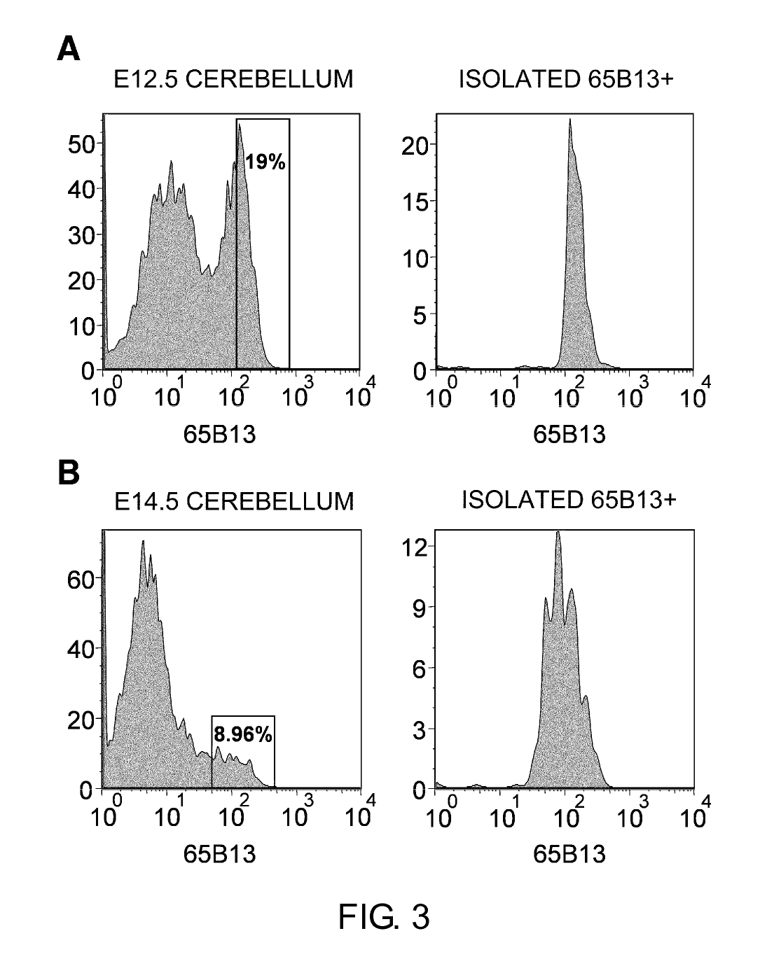

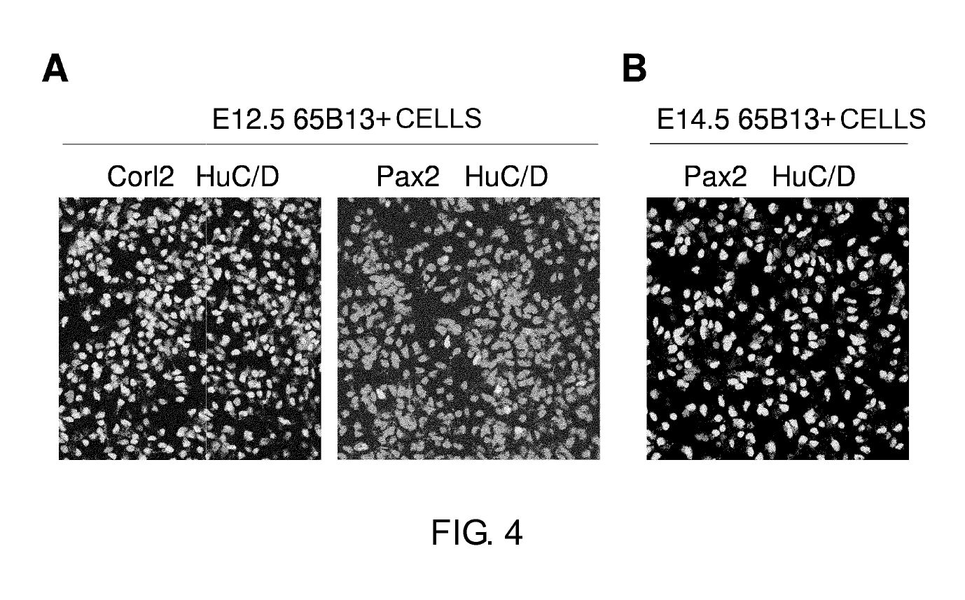

The result showed that 65B13-positive cells could be isolated alive from the cerebellar primordium areas at both of the developmental stages (FIG. 3). Furthermore, it was demonstrated that almost the entire population of the cells differentiated into neurons after two days of culturing, and nearly all of the E12.5 65B13-positive cells differentiated into Cor12-positive Purkinje cells (FIG. 4A) while nearly all of the E14.5 65B13-positive cells differentiated into Pax2-positive Golgi cell-like neurons (FIG. 4B). Thus, it was revealed that in the fetal cerebellum, 65B13 was selectively expressed in progenitor cells of Purkinje and Golgi cells, and these progenitor cells could be separated by using an anti-65B13 antibody. Specifically, 65B13 was demonstrated to be useful as a marker for separating GABA-producing neuron progenitor cells in the cerebellum.

### Example 2

### Specific Expression of Foreign Genes Using the 65B13 Promoter in GABA-Producing Neuron Progenitor Cells

Next, whether foreign genes can be expressed in a GABA-producing neuron progenitor cell-specific manner using the 65B13 promoter was assessed by creating transgenic mice and analyzing the expression of foreign genes according to the protocol described below.

First, the poly A addition sequence of bovine growth hormone (SEQ ID NO: 41; derived from Invitrogen pcDNA3.1+ vector) was amplified and inserted into the HindIII/XhoI site of pSP73 (Promega) to construct pSP73-polyA. Then, the synthetic DNAs of SEQ ID NOs: 42 and 43 were annealed to each other and inserted into the Asp718I/BamHI site of pSP73-polyA to construct pSP73-polyA II. A mouse genomic fragment (SEQ ID NO: 44) located about 3.2 kb upstream of the translation initiation codon of 65B13 was inserted into the ClaI/Asp718I site of pSP73-polyA II to construct pN3. Finally, mouse Gsh1 cDNA (SEQ ID NO: 45) was inserted as a foreign gene into the Asp718I/SalI site of pN3 to construct pN3-Gsh1. After linearized with ClaI, pN3-Gsh1 was injected into the pronuclei of mouse fertilized eggs according to the method of Gordon et al. (Gordon J W, Scangos G A, Plotkin D J, Barbosa J A, Ruddle F H. Genetic transformation of mouse embryos by microinjection of purified DNA. Proc Natl Acad Sci USA. 1980 December; 77(12):7380-4), and the eggs were transplanted into foster mothers. The fetuses were recovered at embryonic day 12.5, and the expression of Neph3 and Gsh1 in the cerebellar primordia was analyzed by the methods described in Example 1. An anti-Gsh1 antibody was prepared by the method described below. First, an expression vector was constructed for a GST fusion protein with amino acids 1 to 72 of Gsh1 as an immunization antigen. After the resulting vector was introduced into E. coli (JM109 strain), the expression was induced with IPTG. The fusion protein was collected using glutathione beads. After the rats were immunized with the collected fusion protein, lymphocytes were collected and fused with myeloma cell P3U1. Thus, anti-Gsh1 antibody-producing hybridomas were obtained (hybridoma preparation was outsourced to Kohjin Bio Co.).

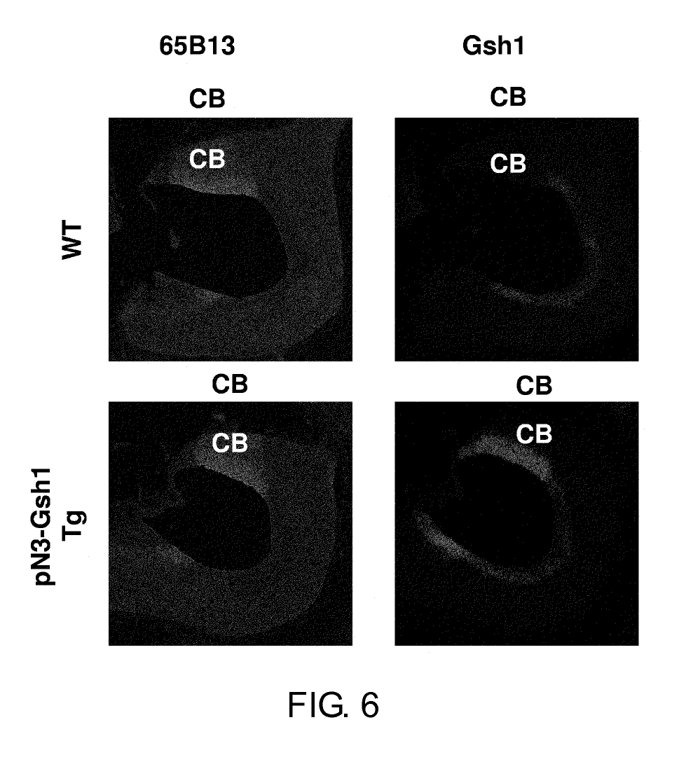

The result showed that the wild-type cerebellum expressed Gsh1 only in a very small ventral fraction of 65B13-positive GABA-producing neuron progenitor cells, while the transgenic mice expressed Gsh1 specifically in the entire 65B13-positive area (FIG. 6). This finding demonstrates that foreign genes can be expressed in a GABA-producing neuron progenitor cell-specific manner by using the 65B13 promoter.

### Example 3

### Identification of Purkinje Progenitor Cell-Specific Genes

The 65B13 gene is known to be expressed transiently in dopamine-producing neuron progenitor cells after termination of cell division (see Patent Document 1); however, there is no report published on the connection between the gene and GABA neuron (WO 2004/038018; Minaki Y, Mizuhara E, Morimoto K, Nakatani T, Sakamoto Y, Inoue Y, Satoh K, Imai T, Takai Y, Ono Y. Migrating postmitotic neural precursor cells in the ventricular zone extend apical processes and form adherens junctions near the ventricle in the developing spinal cord. Neurosci Res. 2005, 52(3):250-62). The cerebellum is constituted of GABA-producing neurons such as Purkinje cells, Golgi cells, stellate cells, and basket cells, and glutamic acid-producing granule cells (Wang V Y, Zoghbi H Y. Genetic regulation of cerebellar development. Nat Rev Neurosci. 2001, 2(7):484-91). The granule cells are known to develop in the rhombic lip region at E12.5 to E14.5 (Wang V Y, Zoghbi H Y. Genetic regulation of cerebellar development. Nat Rev Neurosci. 2001, 2(7):484-91). By contrast, the development of GABA-producing neurons is still poorly understood. However, recent findings suggest that Purkinje cells are generated at E11.5 to E13.5 in the dorsal rhombomere 1 (cerebellar primordium area) (Chizhikov V V, Lindgren A G, Currle D S, Rose M F, Monuki E S, Millen K J. The roof plate regulates cerebellar cell-type specification and proliferation. Development 2006, 133(15):2793-804; Minaki Y, Nakatani T, Mizuhara E, Inoue T, Ono Y. Identification of a novel transcriptional corepressor, Cor12, as a cerebellar Purkinje cell-selective marker. Gene Expr. Patterns, 2008, 8(6):418-423). Although there is no detailed report on Golgi cells, they are assumed to be generated at a late developmental stage (E13.5 to E15.5) in the same region. The stellate cells and basket cells are speculated to originate from progenitor cells in the white matter after birth (Zhang L, Goldman J E. Generation of cerebellar interneurons from dividing progenitors in white matter. Neuron. 1996, 16(1):47-54). Of these neurons, only Purkinje cells transmit neural signals outwardly from the cerebellar cortex. Degeneration of Purkinje cells is involved in the onset of spinocerebellar degeneration and such. 65B13 is a useful marker for isolation of GABA neuron progenitor cells including Purkinje cells. However, it was suggested that to obtain Purkinje progenitor cells of higher purity, Purkinje progenitor cells had to be further purified from a 65B13-positive cell population using a marker that is selectively expressed by Purkinje progenitor cells. Thus, the present inventors identified such selective markers according to the following protocol.

First, cerebellar primordia, myelencephalons, and spinal cords were excised from E12.5 mice, and also cerebellar primordia were excised from E14.5 mice. The cells were dispersed in accumax (MS TECHONOSYSTEMS), and without fixation or permeability treatment, they were stained at 4° C. for 30 minutes using an anti-65B13 monoclonal antibody (100 times diluted purified antibody (described in Minaki et al., 2005) in D-MEM-F12 containing 1% fetal bovine serum and 1 mM EDTA). Then, the cells were washed three times with D-MEM:F12 medium containing 1% fetal bovine serum and 1 mM EDTA at 4° C. for three minutes. After staining with a PE-labeled anti-hamster IgG antibody (BD pharmingen; 8 μg/ml in D-MEM:F12 medium containing 1% fetal bovine serum and 1 mM EDTA) at 4° C. for 30 minutes, the cells were washed in the same way as described above. After staining, 65B13-expressing cells were isolated by a cell sorter. Immediately after cell isolation, total RNA was prepared from the cells using the RNeasy Mini Kit (Qiagen), and double-stranded cDNA was synthesized using a cDNA synthesis kit (TAKARA). Then, the synthesized cDNA was digested with restriction enzyme AfaI (TAKARA). After attaching ad2, the cDNA was amplified by PCR using ad2S as a primer.

The amplification conditions were: five minutes of incubation at 72° C.; and 20 cycles of 94° C. for 30 seconds, 65° C. for 30 seconds, and 72° C. for two minutes; followed by two minutes of final incubation at 72° C.

| | |
|---|---|
| ad2S: CAGCTCCACAACCTACATCATTCCGT | (SEQ ID NO: 54) |
| ad2A: ACGGAATGATGT | (SEQ ID NO: 55) |

PCR was carried out using a reaction solution containing the following components:

| | | |
|---|---|---|
| 10x ExTaq | 5 | μl |
| 2.5 mM dNTP | 4 | μl |
| ExTaq | 0.25 | μl |
| 100 μM primer | 0.5 | μl |
| cDNA | 2 | μl |
| distilled water | 38.25 | μl |

Next, search of the samples was performed using a subtraction (N-RDA) method (described in WO 2004/065599) for genes specific to 65B13-positive cells derived from E12.5 cerebellar primordium. One of the isolated cDNA fragments was found to encode E-cadherin. Then, the expression of E-cadherin was confirmed by the following RT-PCR method using the primers listed below.

PCR was carried out in the following reaction system using as a 3-ng or 0.3-ng aliquot of the amplified cDNA as template.

| | | |
|---|---|---|
| 10x ExTaq | 1 | μl |
| 2.5 mM dNTP | 0.8 | μl |
| ExTaq | 0.05 | μl |
| 100 μM primer | 0.1 | μl each |
| cDNA | 1 | μl |
| distilled water | 6.95 | μl |

After two minutes of incubation at 94° C., PCR amplification was carried out with 26 cycles of 94° C. for 30 seconds, 65° C. for 30 seconds, and 72° C. for two minutes, followed by two minutes of final incubation at 72° C.

PCR was carried out using the following primers:

| E-cadherin: | |
|---|---|
| CTCCAATGCCTGCTCTTGATGGTAGC | (SEQ ID NO: 56) |
| TCTCTGTGTAGCCCTGGCTGTCCTAG | (SEQ ID NO: 57) |
| 65B13: | |
| CTTCCCGTATGCTACCTTGTCTCCAC | (SEQ ID NO: 58) |
| CCAACAGTCCTGCATGCTTGTAATGA | (SEQ ID NO: 59) |

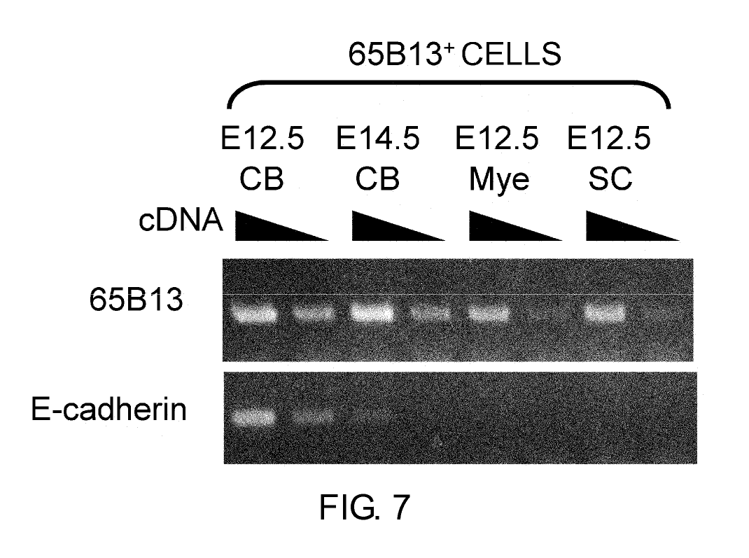

The result showed that among the 65B13-positive cells derived from various brain areas, E-cadherin was specific to 65B13-positive cells derived from the cerebellar primordium which produces Purkinje cells (FIG. 7). It was also demonstrated that E-cadherin was expressed in cerebellar primordium-derived 65B13-positive cells specifically at the stage of Purkinje cell differentiation (E 12.5).

### Example 4

### Expression Analysis of E-Cadherin in Fetal Brain Tissues

Next, to assess the expression of E-cadherin in fetal brain, in particular, 65B13-positive cells, the expression of E-cadherin and 65B13 was compared with the expression of the Purkinje cell-specific marker Cor12 (Minaki Y, Nakatani T, Mizuhara E, Inoue T, Ono Y. Identification of a novel transcriptional corepressor, Cor12, as a cerebellar Purkinje cell-selective marker. Gene Expr. Patterns, 2008, 8(6):418-423) and another GABA neural marker, Pax2, according to the protocol described below.

Brain tissues were excised from day-12.5 and day-14.5 mouse embryos, and fixed in 4% PFA/PBS(−) at 4° C. for two hours and one hour, respectively. After fixation, the solution was replaced with 10% sucrose/PBS(−) at 4° C. for five hours, and then with 20% sucrose/PBS(−) at 4° C. overnight. The tissues were embedded in OCT, and sliced into 14-μm sections. The sections were placed onto glass slides, dried at room temperature for 30 minutes, and again wetted with 0.1% Triton X-100/PBS(−). Then, the tissues were blocked with BlockAce at room temperature for 30 minutes, and incubated with primary antibodies at 4° C. overnight. The glass slides were washed three times with 0.1% TritonX-100/PBS(−) at room temperature for ten minutes. Then, the tissues were incubated with fluorescently labeled secondary antibodies at room temperature for 40 minutes. After washing in the same way as described above, the glass slides were rinsed twice with PBS(−), and the sections were mounted. The primary antibodies used are listed below: 65B13, as described in Example 3; Cor12, as described in Minaki Y, Nakatani T, Mizuhara E, Inoue T, Ono Y. Identification of a novel transcriptional corepressor, Cor12, as a cerebellar Purkinje cell-selective marker. Gene Expr. Patterns, 2008, 8(6):418-423; Pax2, purchased from Zymed (500 times diluted); and E-cadherin, purchased from TAKARA (100 times diluted).

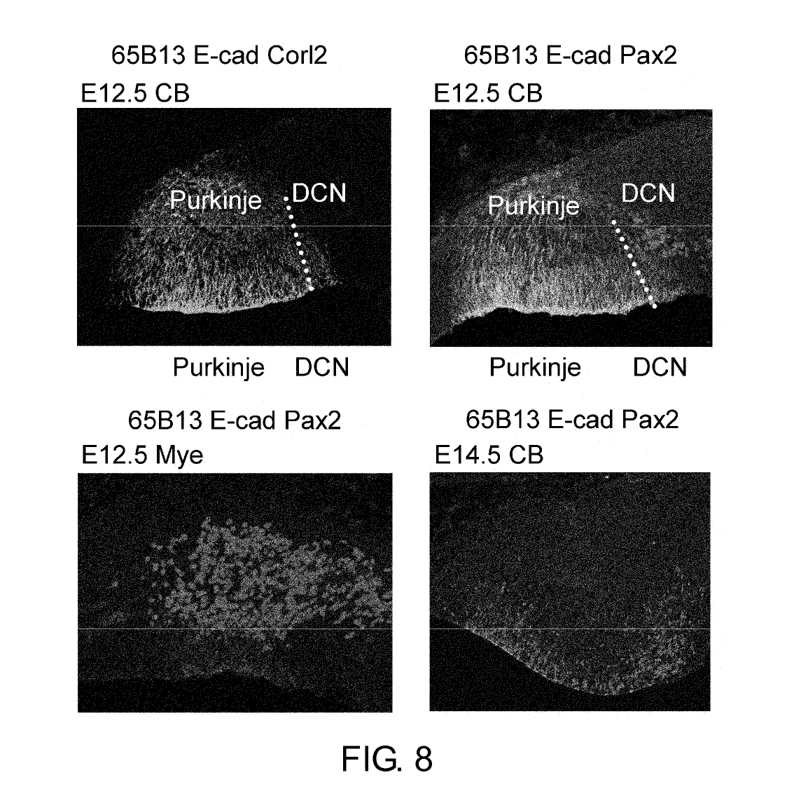

The result showed that E-cadherin was expressed selectively in the cerebellar primordium but not in the myelencephalon (FIG. 8). Furthermore, it was demonstrated that E-cadherin was expressed selectively in the 65B13-positive area of cerebellar primordium and specifically at the stage of Purkinje cell differentiation (E12.5). Furthermore, the expression level of E-cadherin within the 65B13-positive area of E12.5 cerebellar primordium was higher in the dorsal region, from which Cor12-positive Purkinje cells originate, than in the ventral region, where Pax2-positive cerebellar nucleus GABA neurons are generated.

### Example 5

### FACS Analysis of E-Cadherin and 65B13 Expression in Fetal Brain Cells

Next, to confirm that E-cadherin is a useful marker in FACS-based isolation of Purkinje progenitor cells, FACS analysis was carried out according to the protocol described below.

First, cerebellar primordia, myelencephalons, and spinal cords were excised from E12.5 mice, and also cerebellar primordia were excised from E14.5 mice. The cells were dispersed using the NeuroCult Chemical Dissociation Kit (StemCell Technologies Inc), and without fixation or permeability treatment, they were stained at 4° C. for 30 minutes using an anti-65B13 monoclonal antibody (purified antibody described in Example 3; 100 times diluted) or an anti-E-cadherin antibody (100 times diluted). Then, the cells were washed three times with PBS containing 1 mM EDTA and 1% fetal bovine serum at 4° C. for three minutes. After staining with a PE-labeled hamster IgG antibody (BD pharmingen; 8 μg/ml, in PBS containing 1% fetal bovine serum and 1 mM EDTA) or APC-labeled anti-rat IgG antibody (Jackson; 10 μg/ml, in PBS containing 1% fetal bovine serum and 1 mM EDTA) at 4° C. for 30 minutes, the cells were washed in the same way as described above. After staining, E-cadherin-expressing and 65B13-expressing cells were detected using a flow cytometer.

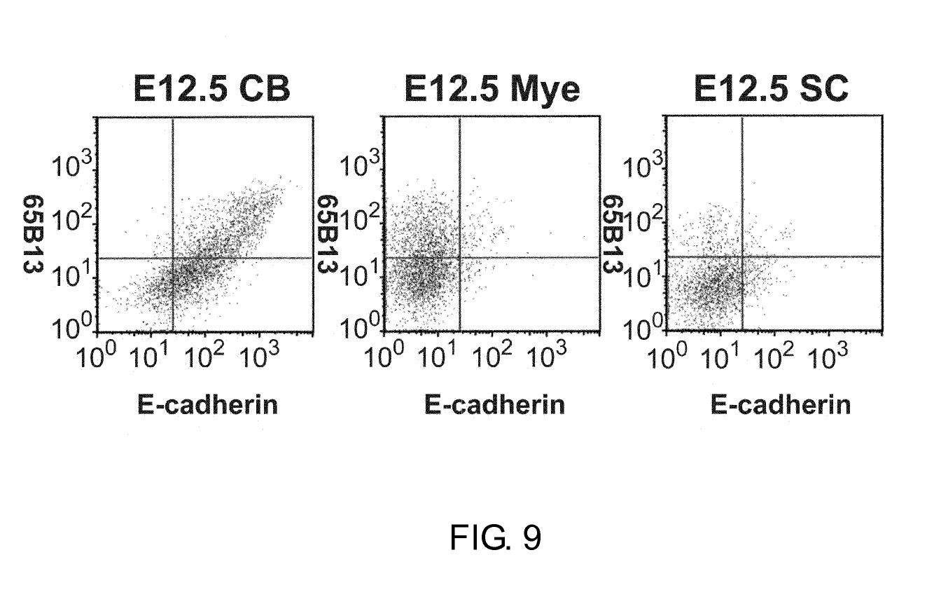

In correlation with the result of tissue expression analysis described in Example 4, the result demonstrates that only cerebellum-derived 65B13-positive cells express E-cadherin on their cell surface (FIG. 9). Thus, E-cadherin was shown to be a useful marker in the FACS-based isolation of Purkinje progenitor cells from a 65B13-positive cell population.

### Example 6

### Isolation and Culture of E-Cadherin and 65B13-Double Positive Cells

Next, to confirm that E-cadherin and 65B13-double positive cells are Purkinje progenitor cells, isolation and culture experiments were carried out according to the protocol described below.

First, cells from cerebellar primordia of E12.5 mice were stained by the same method described in Example 5. E-cadherin and 65B13-double positive cells were isolated using a cell sorter. The isolated cells were placed onto glass slides coated with poly-L-ornithine (Sigma; 0.002% in PBS), laminin (Invitrogen; 2.5 μg/ml in PBS), and fibronectin (Sigma; 5 μg/ml in PBS), and cultured at 37° C. for two days in D-MEM:F12 medium supplemented with N2 (Invitrogen; 1×), B27 (Invitrogen; 1×), and BDNF (Invitrogen; 20 ng/ml). The cultured cells were fixed in 2% PFA/PBS at 4° C. for 20 minutes, and washed twice with PBS at 4° C. for ten minutes. Then, cell permeability treatment was performed using 0.3% Triton X-100/PBS at room temperature for 30 minutes, and the cells were blocked with 10% normal donkey serum/BlockAce at room temperature for 20 minutes. Next, the cells were incubated with a primary antibodies (10% normal donkey serum, 2.5% BlockAce, 0.1% Triton X-100/PBS) at room temperature for one hour and then at 4° C. overnight. On the next day, after the cells were washed three times with 0.1% Triton X-100/PBS at room temperature for ten minutes, they were incubated with fluorescently labeled secondary antibodies (all from Jackson; 3 μg/ml, 10% normal donkey serum, 2.5% BlockAce, 0.1% Triton X-100/PBS) at room temperature for 30 minutes. After washing in the same way as described above, the cells were washed with PBS at room temperature for five minutes, mounted, and observed. The primary antibodies used are listed below: HuC/D, purchased from Molecular Probe; and Cor12, as described in Example 1.

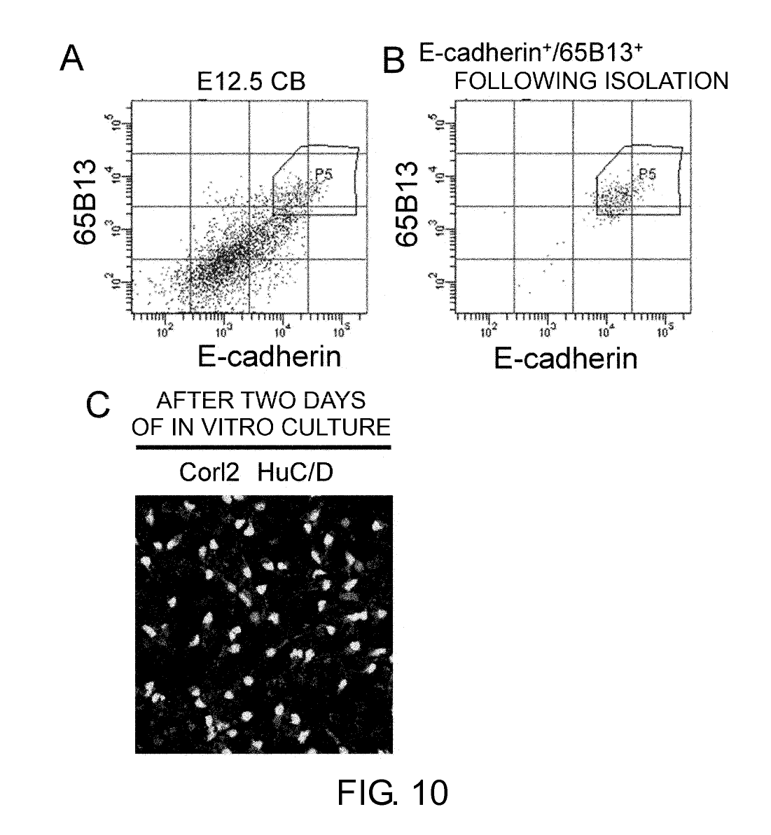

The result showed that most of the isolated E-cadherin and 65B13 double-positive cells differentiated into Cor12-positive Purkinje cells after two days of culture (FIG. 10). Thus, E-cadherin used in combination with 65B13 was demonstrated to serve as a useful maker for isolating Purkinje progenitor cells.

### Sequence Listing Free Text

SEQ ID NO: 19 Mouse 65B13 NM_{—}172898 extracellular: 20-513 a.a.
SEQ ID NO: 21 Mouse 65B13 BC052773 extracellular: 20-513 a.a.
SEQ ID NO: 23 Human 65B13 NM_{—}032123 extracellular: 21-510 a.a.
SEQ ID NO: 25 Human 65B13 NM_{—}199180 extracellular: 21-510 a.a.
SEQ ID NO: 27 Human 65B13 AY358742 extracellular: 21-510 a.a.
SEQ ID NO: 29 Human 65B13 AY305301 extracellular: 21-510 a.a.
SEQ ID NO: 31 Human 65B13 NM_{—}199179 extracellular: 21-460 a.a.
SEQ ID NO: 33 Human 65B13 AY305302 extracellular: 21-460 a.a.
SEQ ID NO: 35 Human 65B13 BC064925 extracellular: 21-490 a.a.
SEQ ID NO: 37 Chimpanzee 65B13 (predicted) XM_{—}512603 extracellular: 21-445 a.a.
SEQ ID NO: 39 Cattle 65B13 (predicted) XM_{—}583222 extracellular: 44-607 a.a.

### SEQ ID NO: 46 Human E-cadherin NM_{—}0043600RF: 125-2773

SEQ ID NO: 47 Human E-cadherin amino acids SS (signal sequence): 1-22 TM (transmembrane domain):709-731

### SEQ ID NO: 48 Mouse E-cadherin NM_{—}009864 ORF: 128-2782

SEQ ID NO: 49 Mouse E-cadherin amino acids SS: 1-22 TM:711-733

### SEQ ID NO: 50 Rat E-cadherin NM_{—}031334 ORF: 127-2787

SEQ ID NO: 51 Rat E-cadherin amino acids SS: 1-22 TM:713-735

### SEQ ID NO: 52 Chimpanzee E-cadherin XM_{—}0011681500RF: 192-2840

SEQ ID NO: 53 Chimpanzee E-cadherin amino acids SS: 1-22 TM:709-731

1. A method for detecting or selecting a Purkinje progenitor cell, which comprises the steps of detecting a protein selected from (A) and (B) below or detecting a marker protein by linking a polynucleotide encoding the marker protein to a promoter that controls expression of an mRNA translated into a protein selected from (A) and (B) below:
(A)
(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and
(B)
(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53.

2. (canceled)

3. The method of claim 1, wherein the step of detecting the protein comprises the steps of:
(d) contacting a test cell sample with an antibody that binds to the protein described in claim 1; and
(e) detecting reactivity.

4. The method of claim 1, wherein the detection step is followed by the step of separating a Purkinje progenitor cell from the detected sample.

5. A method for detecting or selecting a Purkinje progenitor cell, which comprises the steps of
(A) detecting the expression of a polynucleotide capable of hybridizing to a polynucleotide selected from (i), (ii), (iii), and (iv) below, or a complementary sequence thereof:
(i) a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(ii) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iii) a polynucleotide which is expressed in a Purkinje progenitor cell and hybridizes under stringent conditions to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iv) a polynucleotide which is expressed in a Purkinje progenitor cell and has 80% or higher sequence identity to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(B) detecting the expression of a polynucleotide capable of hybridizing to a polynucleotide selected from (i′), (ii′), (iii′), and (iv′) below, or a complementary sequence thereof:
(i′) a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(ii′) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(iii′) a polynucleotide which is expressed in a Purkinje progenitor cell and hybridizes under stringent conditions to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
(iv′) a polynucleotide which is expressed in a Purkinje progenitor cell and has 80% or higher sequence identity to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52.

6. The method of claim 5, wherein the step of detecting the expression of a polynucleotide comprises the steps of:
(a) contacting a test cell sample with a polynucleotide that can hybridize to the polynucleotide described in claim 5 or to a complementary sequence thereof, or with a probe comprising the polynucleotide; and
(b) detecting reactivity.

7. The method of claim 6, wherein the probe is contacted with mRNA prepared from the test cell sample or a complementary DNA (cDNA) transcribed from the mRNA in step (a).

8. The method of claim 5, wherein the step of detecting the expression of a polynucleotide that can hybridize to the polynucleotide described in claim 5 or to a complementary sequence thereof comprises the steps of:
(a-1) conducting gene amplification using a polynucleotide derived from the test cell sample as a template, and a primer comprising a polynucleotide that can hybridize to the polynucleotide described in claim 5 or to a complementary sequence thereof, or a set of primers comprising a polynucleotide that can hybridize to the polynucleotide selected in claim 5 or to a complementary sequence thereof; and
(b-1) detecting the resulting amplification product.

9. The method of claim 8, wherein mRNA prepared from the test cell sample or a complementary DNA (cDNA) transcribed from the mRNA is used as a template in step (a-1).

10. The method of claim 5, wherein the detection step is followed by the step of separating a Purkinje progenitor cell from the detected sample.

11. A kit or reagent for detecting a Purkinje progenitor cell, which comprises a probe, a primer, or a set of primers that enable detection of the expression of or selection of a polynucleotide that can hybridize to a polynucleotide selected from (A) and (B) below, or to a complementary sequence thereof:
(A)
(i) a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(ii) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iii) a polynucleotide which is expressed in a Purkinje progenitor cell and hybridizes under stringent conditions to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iv) a polynucleotide which is expressed in a Purkinje progenitor cell and has 80% or higher sequence identity to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(B)
(i′) a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(ii′) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(iii′) a polynucleotide which is expressed in a Purkinje progenitor cell and hybridizes under stringent conditions to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
(iv′) a polynucleotide which is expressed in a Purkinje progenitor cell and has 80% or higher sequence identity to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52.

12. A kit or reagent for detecting or selecting a Purkinje progenitor cell, which comprises antibodies that bind to proteins described in (A) and (B) below or a polynucleotide comprising a polynucleotide encoding a marker protein linked to a promoter that controls expression of an mRNA translated into a protein selected from (A) and (B) below:
(A)
(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36; and
(B)
(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53.

13. (canceled)

14. A method for producing a Purkinje progenitor cell population, which comprises the steps of:
(VII) obtaining a cell population potentially containing a Purkinje progenitor cell;
(VIII) detecting a Purkinje progenitor cell using the method of claim 1; and
(IX) growing the cell detected or selected in step (VIII).

15. The production method of claim 14, wherein the Purkinje progenitor cell is used to treat cerebellar degeneration.

16. A Purkinje progenitor cell population obtained by the steps of:
(VII) obtaining a cell population potentially containing a Purkinje progenitor cell;
(VIII) detecting or selecting a Purkinje progenitor cell using the method of claim 1; and
(IX) growing the cell detected in step (VIII).

17-19. (canceled)

20. A polynucleotide for detecting or selecting a Purkinje progenitor cell for use in regeneration medicine to treat cerebellar degeneration, which can hybridize to a polynucleotide selected from (A) and (B) below, or to a complementary sequence thereof:
(A)
(i) a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(ii) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iii) a polynucleotide which is expressed in a Purkinje progenitor cell and hybridizes under stringent conditions to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(iv) a polynucleotide which is expressed in a Purkinje progenitor cell and has 80% or higher sequence identity to a polynucleotide comprising:
the nucleotide sequence from positions 178 to 2280 of SEQ ID NO: 1;
the nucleotide sequence from positions 127 to 2079 of SEQ ID NO: 3;
the nucleotide sequence from positions 668 to 2770 of SEQ ID NO: 19;
the nucleotide sequence from positions 130 to 2232 of SEQ ID NO: 21;
the nucleotide sequence from positions 199 to 2100 of SEQ ID NO: 23;
the nucleotide sequence from positions 199 to 2325 of SEQ ID NO: 25;
the nucleotide sequence from positions 15 to 2325 of SEQ ID NO: 27;
the nucleotide sequence from positions 15 to 1916 of SEQ ID NO: 29;
the nucleotide sequence from positions 199 to 1950 of SEQ ID NO: 31;
the nucleotide sequence from positions 15 to 1766 of SEQ ID NO: 33; or
the nucleotide sequence from positions 196 to 2262 of SEQ ID NO: 35;
(B)
(i′) a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(ii′) a polynucleotide which is expressed in a Purkinje progenitor cell and encodes a polypeptide comprising an amino acid sequence with an insertion, substitution, or deletion of one or more amino acids, and/or an addition of one or more amino acids to either or both ends of the amino acid sequence encoded by a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52;
(iii′) a polynucleotide which is expressed in a Purkinje progenitor cell and hybridizes under stringent conditions to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52; and
(iv′) a polynucleotide which is expressed in a Purkinje progenitor cell and has 80% or higher sequence identity to a polynucleotide comprising:
the nucleotide sequence from positions 125 to 2773 of SEQ ID NO: 46;
the nucleotide sequence from positions 128 to 2782 of SEQ ID NO: 48;
the nucleotide sequence from positions 127 to 2787 of SEQ ID NO: 50; or
the nucleotide sequence from positions 192 to 2849 of SEQ ID NO: 52.

21. An antibody for detecting or selecting a Purkinje progenitor cell for use in regeneration medicine to treat cerebellar degeneration, which binds to a protein selected from:
(A)
(v) a protein comprising the amino acid sequence of SEQ ID NO: 2, 4, 20, 22, 24, 26, 28, 30, 32, 34, or 36;
(vi) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 2, 4, 20, 22, 24, 26, 28, 30, 32, 34, or 36;
(vii) a protein which is expressed in a Purkinje progenitor cell and encoded by a polynucleotide that hybridizes under stringent conditions to a polynucleotide encoding the amino acid sequence of SEQ ID NO: 2, 4, 20, 22, 24, 26, 28, 30, 32, 34, or 36; and
(viii) a protein which is expressed in a Purkinje progenitor cell and comprises an amino acid sequence having 80% or higher sequence identity to the amino acid sequence of SEQ ID NO: 2, 4, 20, 22, 24, 26, 28, 30, 32, 34, or 36;
(B)
(v′) a protein comprising the amino acid sequence of SEQ ID NO: 47, 49, 51, or 53;
(vi′) a protein which is expressed in a Purkinje progenitor cell and comprises an amino acid sequence with an insertion, substitution, or deletion of one or more amino acids, and/or an addition of one or more amino acids to either or both ends of the amino acid sequence of SEQ ID NO: 47, 49, 51, or 53;
(vii′) a protein which is expressed in a Purkinje progenitor cell and encoded by a polynucleotide that hybridizes under stringent conditions to a polynucleotide encoding the amino acid sequence of SEQ ID NO: 47, 49, 51, or 53; and
(viii′) a protein which is expressed in a Purkinje progenitor cell and comprises an amino acid sequence having 80% or higher sequence identity to the amino acid sequence of SEQ ID NO: 47, 49, 51, or 53.

22. A method for producing a Purkinje progenitor cell population, which comprises the steps of:
(VII) obtaining a cell population potentially containing a Purkinje progenitor cell;
(VIII) detecting a Purkinje progenitor cell using the method of claim 5; and
(IX) growing the cell detected or selected in step (VIII).

23. The production method of claim 22, wherein the Purkinje progenitor cell is used to treat cerebellar degeneration.

24. A Purkinje progenitor cell population obtained by the steps of:
(VII) obtaining a cell population potentially containing a Purkinje progenitor cell;
(VIII) detecting or selecting a Purkinje progenitor cell using the method of claim 5; and
(IX) growing the cell detected in step (VIII).


SEQUENCE LISTING (59 sequences)

SEQ ID NO: 1 - DNA, 2876 bp, Mus musculus
gatgagccagatttcggggactctgggccagacataaaatcttccagcccggagagaattgtgtgcagagaggggctccagtccagcgtggtgtgagaggcgtgctatcaagaaagaagttggaggggaaccagtgcaaccctaactctacgagatcttggggtacacacactcgggatgctggcctccgccctcctcgttttcctttgctgtttcaaaggacatgcaggctcatcgccccatttcctacaacagccagaggacatggtggtgctgttgggggaggaagcccggctgccctgcgctctgggcgcgtacagggggctcgtgcagtggactaaggatgggctggctctagggggcgaaagagaccttccagggtggtcccggtactggatatcggggaattcagccagtggccagcatgacctccacattaagcctgtggaattggaagatgaggcatcgtatgagtgccaggcttcgcaagcaggtctccgatcacgaccagcccaactgcacgtgatggtccccccagaagctccccaggtactaggcggcccctctgtgtctctggttgctggagttcctggaaatctgacctgtcggagtcgtggggattcccgacctgcccctgaactactgtggttccgagatgggatccggctggatgcgagcagcttccaccagaccacgctgaaggacaaggccactggaacagtggaaaacaccttattcctgaccccttccagtcatgatgatggcgccaccttgatctgcagagcgcgaagccaggccctgcccacagggagggacacagctgttacactgagccttcagtatcccccaatggtgactctgtctgctgagccccagactgtgcaggagggagagaaggtgactttcctgtgtcaagccactgcccagcctcctgtcactggctacaggtgggcgaaggggggatccccggtgctcggggcacgtgggccaaggttggaggtcgttgcagatgccactttcctgactgagccggtgtcctgcgaggtcagcaacgcggtcggaagcgccaaccgcagcacggcgctggaagtgttgtatggacccattctgcaggcaaaacctaagtccgtgtccgtggacgtggggaaagatgcctccttcagctgtgtctggcgcgggaacccacttccacggataacctggacccgcatgggtggctctcaggtgctgagctccgggcccacgctgcggcttccgtccgtggcactggaggatgcgggcgactatgtatgcagggctgagccgaggagaacgggtctgggaggcggcaaagcgcaggcgaggctgactgtgaacgcaccccctgtagtgacagccctgcaacctgcaccagcctttctgaggggtcctgctcgcctccagtgtgtggtgtttgcctcccctgccccagactcggtggtttggtcttgggacgagggcttcttggaggcaggctcactgggcaggttcctagtggaagccttcccagccccggaagtggaggggggacagggccctggccttatttctgtgctacacatttccggaacccaggagtccgactttaccaccggcttcaactgcagtgcccgcaaccggctaggagagggacgagtccagatccacttgggccgtagagatttgctgcctactgtccggattgtggctggtgcagcatctgcagccacctctctccttatggtcatcactggagtggtcctctgctgctggcgccatggctctctctctaagcaaaagaacttggtccggatcccaggaagcagcgagggttccagttcacgtggccctgaggaggagacaggcagcagtgaggaccggggtcccattgtgcacaccgaccacagtgatttggttcttgaggaaaaagaggctctggagacaaaggatccaaccaacggttactacaaggttcgaggggtcagtgtgagccttagccttggggaagctcctggaggaggcctcttcttgccaccgccctctccgatcggtctcccagggactcctacttactatgacttcaagccacatctggacttagtccctccctgcagactgtacagagcgagggcaggttatcttaccaccccccatccccgtgccttcaccagctacatgaaacccacatcctttggacccccagatttgagctctggaactccccccttcccgtatgctaccttgtctccacccagccaccagcgtctccagactcatgtgtgaatccatctctccaagtgaagggtcttggaatcttctgtttgccatatagtgtgttgtccagatttctggggagtcagaacaagttgatgaccaacccctccaaaactgaacattgaaggagggaaagatcattacaagcatcaggactgttggtgtacactcagttcagccaaagtggattctccaagtgggagcaatatggccgctttcccatgagaaagacattcaagatggtgactaaatgactaaatactttgcagagggacaaagatgggaactagggatacggatggaagtagtagagaagatatatgaccatctgcatcaagaggaaggataacatatgacaaatcaagatgaaagaaataatccaccccacccccaccgcgtcctggccaataagtatagcctacatggctgttcattatctgggaaccaaaatggccactatcttgactccttccttaaagatacagaaagaattgaatccaaggaatggggtagggtggaaatagaagaaatgaaggggactcttgggctaagaatacttatgtttaataataaaagggggaggcaaagatgcaaaaaaaaaaaaa

SEQ ID NO: 2 - protein, 700 aa, Mus musculus
MLASALLVFLCCFKGHAGSSPHFLQQPEDMVVLLGEEARLPCALGAYRGLVQWTKDGLALGGERDLPGWSRYWISGNSASGQHDLHIKPVELEDEASYECQASQAGLRSRPAQLHVMVPPEAPQVLGGPSVSLVAGVPGNLTCRSRGDSRPAPELLWFRDGIRLDASSFHQTTLKDKATGTVENTLFLTPSSHDDGATLICRARSQALPTGRDTAVTLSLQYPPMVTLSAEPQTVQEGEKVTFLCQATAQPPVTGYRWAKGGSPVLGARGPRLEVVADATFLTEPVSCEVSNAVGSANRSTALEVLYGPILQAKPKSVSVDVGKDASFSCVWRGNPLPRITWTRMGGSQVLSSGPTLRLPSVALEDAGDYVCRAEPRRTGLGGGKAQARLTVNAPPVVTALQPAPAFLRGPARLQCVVFASPAPDSVVWSWDEGFLEAGSLGRFLVEAFPAPEVEGGQGPGLISVLHISGTQESDFTTGFNCSARNRLGEGRVQIHLGRRDLLPTVRIVAGAASAATSLLMVITGVVLCCWRHGSLSKQKNLVRIPGSSEGSSSRGPEEETGSSEDRGPIVHTDHSDLVLEEKEALETKDPTNGYYKVRGVSVSLSLGEAPGGGLFLPPPSPIGLPGTPTYYDFKPHLDLVPPCRLYRARAGYLTTPHPRAFTSYMKPTSFGPPDLSSGTPPFPYATLSPPSHQRLQTHV

SEQ ID NO: 3 - DNA, 2243 bp, Mus musculus
gagagaattgtgtgcagagagaggctccagtccagcgtggtgtgagaggcgtgctatcaagaaagaagttggaggggaaccagtgcaaccctaactctacgagatcttggggtacacacactcgggatgctggcctccgccctcctcgttttcctttgctgtttcaaaggacatgcagggtggtcccggtactggatatcggggaattcagccagtggccagcatgacctccacattaagcctgtggaattggaagatgaggcatcgtatgagtgccaggcttcgcaagcaggtctccgatcacgaccagcccaactgcacgtgatggtccccccagaagctccccaggtactaggcggcccctctgtgtctctggttgctggagttcctggaaatctgacctgtcggagtcgtggggattcccgacctgcccctgaactactgtggttccgagatgggatccggctggatgcgagcagcttccaccagaccacgctgaaggacaaggccactggaacagtggaaaacaccttattcctgaccccttccagtcatgatgatggcgccaccttgatctgcagagcgcgaagccaggccctgcccacagggagggacacagctgttacactgagccttcagtatcccccaatggtgactctgtctgctgagccccagactgtgcaggagggagagaaggtgactttcctgtgtcaagccactgcccagcctcctgtcactggctacaggtgggcgaaggggggatccccggtgctcggggcacgtgggccaaggttggaggtcgttgcagatgccactttcctgactgagccggtgtcctgcgaggtcagcaacgcggtcggaagcgccaaccgcagcacggcgctggaagtgttgtatggacccattctgcaggcaaaacctaagtccgtgtccgtggacgtggggaaagatgcctccttcagctgtgtctggcgcgggaacccacttccacggataacctggacccgcatgggtggctctcaggtgctgagctccgggcccacgctgcggcttccgtccgtggcactggaggatgcgggcgactatgtatgcagggctgagccgaggagaacgggtctgggaggcggcaaagcgcaggcgaggctgactgtgaacgcaccccctgtagtgacagccctgcaacctgcaccagcctttctgaggggtcctgctcgcctccagtgtgtggtgtttgcctcccctgccccagactcggtggtttggtcttgggacgagggcttcttggaggcaggctcactgggcaggttcctagtggaagccttcccagccccggaagtggaggggggacagggccctggccttatttctgtgctacacatttccggaacccaggagtccgactttaccaccggcttcaactgcagtgcccgcaaccggctaggagagggacgagtccagatccacttgggccgtagagatttgctgcctactgtccggattgtggctggtgcagcatctgcagccacctctctccttatggtcatcactggagtggtcctctgctgctggcgccatggctctctctctaagcaaaagaacttggtccggatcccaggaagcagcgagggttccagttcacgtggccctgaggaggagacaggcagcagtgaggaccggggtcccattgtgcacaccgaccacagtgatttggttcttgaggaaaaagaggctctggagacaaaggatccaaccaacggttactacaaggttcgaggggtcagtgtgagccttagccttggggaagctcctggaggaggcctcttcttgccaccgccctctccgatcggtctcccagggactcctacttactatgacttcaagccacatcaggacttagtccctccctgcagactgtacagagcgagggcaggttatcttaccaccccccatccccgtgccttcaccagctacatgaaacccacatcctttggacccccagatttgagctctggaactccccccttcccgtatgctaccttgtctccacccagccaccagcgtctccagactcatgtgtgaatccatctctccaagtgaagggtcttggaatcttctgtttgccatatagtgtgttgtccagatttctggggagtcagaacaagttgatgaccaacccctccaaaactgaacattgaaggagggaaagatcattacaagcatcaggactgttggtgtacactcag

SEQ ID NO: 4 - protein, 650 aa, Mus musculus
MLASALLVFLCCFKGHAGWSRYWISGNSASGQHDLHIKPVELEDEASYECQASQAGLRSRPAQLHVMVPPEAPQVLGGPSVSLVAGVPGNLTCRSRGDSRPAPELLWFRDGIRLDASSFHQTTLKDKATGTVENTLFLTPSSHDDGATLICRARSQALPTGRDTAVTLSLQYPPMVTLSAEPQTVQEGEKVTFLCQATAQPPVTGYRWAKGGSPVLGARGPRLEVVADATFLTEPVSCEVSNAVGSANRSTALEVLYGPILQAKPKSVSVDVGKDASFSCVWRGNPLPRITWTRMGGSQVLSSGPTLRLPSVALEDAGDYVCRAEPRRTGLGGGKAQARLTVNAPPVVTALQPAPAFLRGPARLQCVVFASPAPDSVVWSWDEGFLEAGSLGRFLVEAFPAPEVEGGQGPGLISVLHISGTQESDFTTGFNCSARNRLGEGRVQIHLGRRDLLPTVRIVAGAASAATSLLMVITGVVLCCWRHGSLSKQKNLVRIPGSSEGSSSRGPEEETGSSEDRGPIVHTDHSDLVLEEKEALETKDPTNGYYKVRGVSVSLSLGEAPGGGLFLPPPSPIGLPGTPTYYDFKPHQDLVPPCRLYRARAGYLTTPHPRAFTSYMKPTSFGPPDLSSGTPPFPYATLSPPSHQRLQTHV

SEQ ID NO: 5 - DNA, 2750 bp, Mus musculus
aacagccaggagcagtgaccgagccgctggagctggggagagacgcgcggaagactgggccaggagactagggaccgagggacgcgcgcctggggagagccaacaaggaacccgcgggccggacagcgacaccggcaatccgcgccaaactgttccagccgctggccttctatagccgcagccccaggacattctaaagctctccaagacgccccctcccctggcttctcgcgttgaccaaggaaaagaaaaagggatggaaaaagaaaggaaggagactagaaagaaaacccagatttgccaccgcacaaaaagagaggtgggggggacaaggaaaaaaaaaaaagtcgagcgactgtggggccggaacacaggcagcgggatcgtgggccgagcgatgcaaggctgcgcgcccaagcggccgcgagttgtgactgaagccaggatgctcgtccaggcgcagtgaagagccagaccgtgttgcctccccaggagtccaagcgcagggagggccgctcggaggacgcggcagactgcctggcaggccaccggccgaggtgacagggctggggcggtggggagcgagcgagtgcgcccggctgcgtccgcccgaagcggacggtccctttccatttttgactggcacaaaaaagaaaactctccaaaggggtgggggctacctaagcaacaactacaatcaacaaaatatcctacccaacccgccatctcccccacacctcggtctgcccccgccccctccccaggcccagcgcgggcgcccagagcgtcccaactcactgcaagaaaccggcaatgtaggatccaaagctttctactcccgtgttcttttctttccgtgttttttttaaaggggaaaacccggtggtgggcagtctgacacgcacacaacctgccttcatactctgacaaaagcagatgcactttgacttctgacagctctacctcaagctggagagaacccagctttcccgaatcctgagctcttggcgtcttccttttcgtctgtttccattttatttatttacgtcccgccgcctctcacggtgaccttcactccttcgcgggctttgagcagaagagccgctttctagcccgcttgagactgattttcctcgcccggtgagctgaggtggcgctgctccatcccgttgccccgggactccggggctgccctctaccagcctggtctctcccccttttgatttgctagtacgggttttttgcttgcccaactagagagggtttcttctttttggaggagctggttgtcttcagaagtcatcccctcgactctaattgccctgtcgctccgggcctcaccggaccaaaccaaagaccatggtgcactgtgcgggctgcaaaaggcccatcctggaccgtttcctcttgaacgtgttggacagggcctggcacgtcaagtgcgtccagtgctgtgaatgtaaatgcaacctgaccgagaagtgcttctcccgggaaggcaagctctactgtaaaaacgacttcttccgatgtttcggtaccaaatgcgccggttgtgcgcagggcatctctccaagcgatctggttcgcagagcgcgaagcaaagtgtttcacctcaactgcttcacctgcatgatgtgtaacaagcagctctccaccggcgaggagctctacatcatagacgagaacaagttcgtttgtaaagaggattacctgagtaacagcagtgtcgccaaagagaacagcctccactcggccaccacaggcagtgaccctagtttatctccggattcccaagatccatcgcaggatgatgccaaggactctgaaagtgccaacgtctcagataaggaaggtggtagtaatgagaatgatgatcagaacctaggtgccaaacgtaggggaccccggaccacgatcaaagccaagcaactggagacgttgaaggcagcctttgcagctacacccaagcccacacgccatatccgtgagcaactggcccaggagactggcctcaacatgcgtgttatccaggtctggttccagaatcgacgctccaaggagcgaaggatgaaacagctaagcgcgctaggcgcgcggcgccacgcctttttccgcagtcctcgtcggatgcggccgctggtggaccgcctggagccgggcgaactcatccccaacggccccttctccttttacggagattaccagagtgagtactacggtcccggaggcaactacgacttcttcccgcaaggaccgccatcctctcaggctcagacgccagtggacctaccctttgtgccatcatctggcccttcggggacgccccttggaggtctggaccacccgctgcctggtcaccacccttccagtgaggcgcagcgatttactgacatcctggcacatcccccaggggactcccctagtcctgagcccagcttgcccgggcctctccactccatgtcagcggaggtcttcgggcccagtccacctttctcatctctgtcggtcaatggtggagccagctacgggaaccatttgtctcaccctcctgaaatgaacgaggcagccgtgtggtagcggggtctcgcatgggccacgggagctcgtggttgtacagagacgagcttttatttcagaaaaatagattaaaaagacaaaaaaaaaaaaacccccaaaacaaaaaagcaagcctcctgctccacttccttcagcctcggggaccagtctgtttggggagactggatagc

SEQ ID NO: 6 - protein, 406 aa, Mus musculus
MVHCAGCKRPILDRFLLNVLDRAWHVKCVQCCECKCNLTEKCFSREGKLYCKNDFFRCFGTKCAGCAQGISPSDLVRRARSKVFHLNCFTCMMCNKQLSTGEELYIIDENKFVCKEDYLSNSSVAKENSLHSATTGSDPSLSPDSQDPSQDDAKDSESANVSDKEGGSNENDDQNLGAKRRGPRTTIKAKQLETLKAAFAATPKPTRHIREQLAQETGLNMRVIQVWFQNRRSKERRMKQLSALGARRHAFFRSPRRMRPLVDRLEPGELIPNGPFSFYGDYQSEYYGPGGNYDFFPQGPPSSQAQTPVDLPFVPSSGPSGTPLGGLDHPLPGHHPSSEAQRFTDILAHPPGDSPSPEPSLPGPLHSMSAEVFGPSPPFSSLSVNGGASYGNHLSHPPEMNEAAVW

SEQ ID NO: 7 - DNA, 2283 bp, Homo sapiens
cggccgcgagttgtgactggagccacgatgcacggccaggcgcggtgagaagccagcccgtagtgcctcccgaaggagcccgggcgcagggagggtcgccctgaggacacggaggccgccaggcaggccaagggccgaggtgactgggctggggcggtagggaaggagcgagtgcgcctggctgcctccgcacggagttgtccctctctgttttcgattgacacaaacacttctccaaaagcggggaaacctaagcaacaacagcaatcaacaccaagatcttcctcctaccctcccctctttcccttctcccgcggtcggccctcgccccctcccccaggcccagcgcgggcgctcggcgcgtccagacccgcggcgcgatgccggcagtttaggatccaaagcttctctgctccttttgttctttccttcccttttttaaaaaaagaggggggaaatcccagtggtgggcagcctggcacgcacacagtcgccctcataccccgacaaaagcagatgcactttgacttctgacagctctacctcaagccccggagaactcagcggcgctttcctcgcaacccgagctcggcgagtcgtcgtcttcttcttctccgtttttatttatttatttccgttcccgccgccgttctcgctgaccttcactcctccgcgggctctgagcagaagggtcgcattctctcccgcctgagacttcttttcctcgccccgggagctcaggcggcgccgctccagcccggggccccgggactccccggctgcacacttcactgagacgcccccccaggccccgatcagcctcgtttcctccaccctactttgatttcctggtgcgagttttggcttgcacggccgagtgtgtgtcctctttttggagagactggggagctcgtgccgattgtcttcaggagtcatcccctgggctctactttgcccctctctctctctgggcctcatcagaccaaaccaaagaccatggttcactgtgccggctgcaaaaggcccatcctggaccgctttctcttgaacgtgctggacagggcctggcacgtcaagtgcgtccagtgctgtgaatgtaaatgcaacctgaccgagaagtgcttctccagggaaggcaaactctactgcaagaacgacttcttccggtgtttcggtaccaaatgcgcaggctgcgctcagggcatctcccctagcgacctggtgcggagagcgcggagcaaagtgtttcacctgaactgcttcacctgcatgatgtgtaacaagcagctctccactggcgaggaactctacatcatcgacgagaataagttcgtctgcaaagaggattacctaagtaacagcagtgttgccaaagagaacagccttcactcggccaccacgggcagtgaccccagtttgtctccggattcccaagacccgtcgcaggacgacgccaaggactcggagagcgccaacgtgtcggacaaggaagcgggtagcaacgagaatgacgaccagaacctgggcgccaagcggcggggaccgcgcaccaccatcaaagccaagcagctggagacgctgaaggccgccttcgctgctacacccaagcccacccgccacatccgcgagcagctggcgcaggagaccggcctcaacatgcgcgtcattcaggtctggttccagaaccggcgctccaaggagcggaggatgaagcagctgagcgccctgggcgcccggcgccacgccttcttccgcagtccgcgccggatgcggccgctggtggaccgcctggagccgggcgagctcatccccaatggtcccttctccttctacggagattaccagagcgagtactacgggcccgggggcaactacgacttcttcccgcaaggccccccgtcctcgcaggcccagacaccagtggacctacccttcgtgccgtcatctgggccgtccgggacgcccctgggtggcctggagcacccgctgccgggccaccacccgtcgagcgaggcgcagcggtttaccgacatcctggcgcacccacccggggactcgcccagccccgagcccagcctgcccgggcctctgcactccatgtcggccgaggtcttcggacccagcccgcccttctcgtcgctgtcggtcaacggtggggcgagctacggaaaccacctgtcccacccccccgaaatgaacgaggcggccgtgtggtagcggggtctcgcacggtctgcggagttcgtggttgtacagaaatgaacctttatttaagaaaaatag

SEQ ID NO: 8 - protein, 406 aa, Homo sapiens
MVHCAGCKRPILDRFLLNVLDRAWHVKCVQCCECKCNLTEKCFSREGKLYCKNDFFRCFGTKCAGCAQGISPSDLVRRARSKVFHLNCFTCMMCNKQLSTGEELYIIDENKFVCKEDYLSNSSVAKENSLHSATTGSDPSLSPDSQDPSQDDAKDSESANVSDKEAGSNENDDQNLGAKRRGPRTTIKAKQLETLKAAFAATPKPTRHIREQLAQETGLNMRVIQVWFQNRRSKERRMKQLSALGARRHAFFRSPRRMRPLVDRLEPGELIPNGPFSFYGDYQSEYYGPGGNYDFFPQGPPSSQAQTPVDLPFVPSSGPSGTPLGGLEHPLPGHHPSSEAQRFTDILAHPPGDSPSPEPSLPGPLHSMSAEVFGPSPPFSSLSVNGGASYGNHLSHPPEMNEAAVW

SEQ ID NO: 9 - DNA, 2405 bp, Mus musculus
aggacacctgctcgaagctggagcgagcgcccggtcgcgacccgtgacatgaggctgtgaccgctgccgccctccacgccactctgggcagtgcagcgccaggccggagagcgtcggaggacttgaccccgagaagtcttggttgatccgtaacggactcgcccctacagactcgcctacagactaggaaggctgagagccacagcagcggggaccgagagggcctaagggcccaggggccccaaggaggacgaggcggcccgagccgccggggcgcgcggctatgatggtgcactgtgctggctgtgagcggcccatcctcgaccgctttctgctgaacgtactagaccgcgcgtggcatatcaaatgtgttcaatgctgcgagtgcaaaaccaacctctcggagaagtgcttctcacgggaaggcaagctatactgtaaaaacgactttttcaggcgctttggcacaaagtgcgccggctgcgcgcaaggtatctctccgagcgacctggtacggaaggcccggagcaaagtcttccacctcaactgcttcacctgtatggtgtgcaataagcagctatccaccggagaggagctctacgtgatcgacgagaacaagtttgtgtgcaaggacgactacttaagctcctctagcctcaaggaaggaagtctcaactcggtgtcgtcctgtacggaccgcagtttgtccccggacctccaggatccgttacaggacgaccccaaagagaccgacaattcgacctcatcggacaaggaaaccgctaacaacgagaatgaggaacagaactccggcaccaaacggcgcggcccgcgcaccaccatcaaggccaagcagctggagacgctcaaggcagccttcgcagccacgcccaagcccacgcgccacattcgcgaacagctggcacaggagacgggcctcaacatgagggtcattcaggtgtggtttcagaaccgaaggtccaaagaacgccgcatgaaacagctgagcgctctgggcgcgcggagacacgccttcttccggagtccgcggcgcatgcgtcccctgggcggccgcttggacgagtctgagatgttggggtctaccccatacacttattacggagactaccaaagtgactactacgctccgggaggcaattacgatttcttcgcgcacggtccgccgtcacaggcgcagtcccctgccgactccagcttcctggcagcatcgggacctggctcgacgccgctgggcgcgctggaaccgccgctggccgggcctcacggcgcggacaaccccagattcaccgacatgatctcgcatccggacacgccgagccccgagccgggcctgcccggtgcgctgcaccccatgccgggagaggtgttcagcggcgggcccagcccgcccttccccatgagcggcaccagcggctacagtggacccctgtcgcaccccaaccctgagctcaacgaagcggccgtatggtaaggccgaggggctgagttgtccccctgccaccaagcccgggacgggacgccgcctgggtaagcctcaagagtcctctcgtgggttcgcacccaaccaggccactcgcatcaccacccctcagagctttggcacgcgcctgcgcaatttctcgggaccaaagtcaatattctgaagggtcgagattccaagcacatcttagaagccctccggatcccccacccatcatcacctccttgaactaagagagggggatgaggccaaggagcggagaccatggcactacccctccctgcgagccgaggcattgtgaaatcctatttctcactttctcttttaaaaaaagaaagaaagaaggaaggaaggaaggaaggaaagaaagaaagagaggttgaaagggggagagaaagagagagagagagagagagagagagagagagagagagagagagagagagagagagagagagagagagagagagagagagagagcgcgagcgagctgaggaaagctcagccagagaagaaaaatgaggaggaccgccggtgaatggtggctttgcaggaagacaaatccacctgcgagttggcgccccctggtggctcaatgtcagcttgtctaggaaggtgcgcggtctgggcctggccttcctgagcccaactccctgctcctccatactttgagtctgaggggcctggcaggattcgaaccctcccaccctgctctgaccctgagccgggcgccaagtcattgagcatttgcccacggatcactctccctgcccgggacctggaggctgggccatcaggacgaacagtattatacttttttggaagtcggacgcttctagtttccttattttgtataaagaagaaacaaataaagtatgtttttgtgaaaaaaaaaaaaaaaaaa

SEQ ID NO: 10 - protein, 402 aa, Mus musculus
MMVHCAGCERPILDRFLLNVLDRAWHIKCVQCCECKTNLSEKCFSREGKLYCKNDFFRRFGTKCAGCAQGISPSDLVRKARSKVFHLNCFTCMVCNKQLSTGEELYVIDENKFVCKDDYLSSSSLKEGSLNSVSSCTDRSLSPDLQDPLQDDPKETDNSTSSDKETANNENEEQNSGTKRRGPRTTIKAKQLETLKAAFAATPKPTRHIREQLAQETGLNMRVIQVWFQNRRSKERRMKQLSALGARRHAFFRSPRRMRPLGGRLDESEMLGSTPYTYYGDYQSDYYAPGGNYDFFAHGPPSQAQSPADSSFLAASGPGSTPLGALEPPLAGPHGADNPRFTDMISHPDTPSPEPGLPGALHPMPGEVFSGGPSPPFPMSGTSGYSGPLSHPNPELNEAAVW

SEQ ID NO: 11 - DNA, 2084 bp, Homo sapiens
aaccaggtacaagctaatactcaacaatactgatgccttgttttttttgctctgtccggacagcaacgctgtagccaatttagatatgctataaatttaagaggttgccatggccacggtgcgcccattggccgctgggccccctacgtgcagcgccacgtcaccaaatctgaataaggatgcgcgaattacgcggcgaccagacaaagatgaggatccggaccgcttgaaagtgggggaaagtgccggcgcctccgccaccggggaaagccgctccgcagcgccgaggccagcagccacccgagatacctggggaagcccggaacaggcgccggggcgtgcggcccgtggcatgaggttgtgaacgccacccgccccccaccaccccactccgggcagcccagcgccaggccagagattgcccaaggactggaccggctgagtcttggtccggaccagactcgccctgcagctgctgagacaagaggcgaagggcagcggagggcccggcaggcccgagggccaggggcccaaagggagggcaaggcggccgaagccgccggggcgcggggctatgatggtgcactgcgccggttgcgagcggcccatcctcgaccgctttctgctgaacgtgctggaccgcgcgtggcacatcaaatgtgttcagtgctgcgagtgcaaaaccaacctctcggagaagtgcttctcgcgcgagggcaagctctactgcaaaaatgactttttcaggcgctttggcacgaaatgcgccggctgcgcgcaaggcatctcgcccagcgacctggtgcgcaaggcccggagcaaagtctttcacctcaactgtttcacctgcatggtgtgtaacaagcagctgtccaccggcgaggagctctacgtcatcgacgagaacaagttcgtgtgcaaagacgactacctgagctcatccagcctcaaggagggcagcctcaactcagtgtcatcctgtacggaccgcagtttgtccccggacctccaggacgcactgcaggacgaccccaaagagacggacaactcgacctcgtcggacaaggagacggccaacaacgagaacgaggagcagaactcgggcaccaagcggcgcggcccccgcaccaccatcaaggccaagcagctggagacgctcaaggctgccttcgccgccacgcccaagcccacgcgccacatccgcgagcagctggcgcaggagaccggcctcaacatgcgcgtcatccaggtgtggtttcagaaccgacggtccaaagaacgccggatgaaacagctgagcgccctaggcgcccggaggcacgccttcttccggagtccgcggcgcatgcgtccgctgggcggccgcttggacgagtctgagatgttggggtccaccccgtacacctactacggagactaccaaggcgactactacgcgccgggaagcaactacgacttcttcgcgcacggcccgccttcgcaggcgcagtccccggccgactccagcttcctggcggcctctggccccggctcgacgccgctgggagcgctggaaccgccgctcgccggcccgcacgccgcggacaaccccaggttcaccgacatgatctcgcacccggacacaccgagccccgagccaggcctgccgggcacgctgcaccccatgcccggcgaggtattcagcggcgggcccagcccgcccttcccaatgagcggcaccagcggctacagcggacccctgtcgcatcccaaccccgagctcaacgaagccgccgtgtggtaaggccgccgggccgccccccgcgctcggcccccgggggccccgccccgaagcagcctcctgaaaccaaaacgcccgacgcagacgcggtgggagacgtgggtgtccctcgggggttctctctcgggtccgcactcaactggcagctgctcctcggctgggcgccgagggggggccgacccccatctccaccccgcgggctctccaggagcctcagcccaccgccagtactctcccagcaaccgcgagcaatttcttgggaccaaagtcaatactccggagggtcaagagatttcgagcacgc

SEQ ID NO: 12 - protein, 402 aa, Homo sapiens
MMVHCAGCERPILDRFLLNVLDRAWHIKCVQCCECKTNLSEKCFSREGKLYCKNDFFRRFGTKCAGCAQGISPSDLVRKARSKVFHLNCFTCMVCNKQLSTGEELYVIDENKFVCKDDYLSSSSLKEGSLNSVSSCTDRSLSPDLQDALQDDPKETDNSTSSDKETANNENEEQNSGTKRRGPRTTIKAKQLETLKAAFAATPKPTRHIREQLAQETGLNMRVIQVWFQNRRSKERRMKQLSALGARRHAFFRSPRRMRPLGGRLDESEMLGSTPYTYYGDYQGDYYAPGSNYDFFAHGPPSQAQSPADSSFLAASGPGSTPLGALEPPLAGPHAADNPRFTDMISHPDTPSPEPGLPGTLHPMPGEVFSGGPSPPFPMSGTSGYSGPLSHPNPELNEAAVW

SEQ ID NO: 13 - DNA, 1914 bp, Rattus norvegicus
cggcgcctccgccaaccgggaaagccgctcgccagtgccgaagccagcagcctccgaggacacctgctcgaagctggagcgagcgcccagacgcgacccgtgacatgaggctgtgaccgccgccgccctccacgccactctgggcagtgcagtgccaggccggagagcgtcggaggacttgaccccgagaagtcttggttgatctgtatcggactcgaccctaccgaagaccacagaccaggggggctgagagccacagaccaggggggctgagagccacagcagcggggaccgagaggccctaagggcccaggggccccaaggaggacgaggcggcccgagccgccggggcgcgcggctatgatggtgcactgtgccggctgtgagcggcccatcctcgaccgctttctgctgaacgtgctggaccgcgcgtggcatatcaaatgtgttcaatgctgcgagtgcaaaaccaacctctcggagaagtgtttctcccgggagggcaagctgtactgtaaaaacgacttcttcaggcgctttggcacaaagtgcgccggctgtgcgcaaggtatctctcccagcgacctggtgcgcaaggcccggagcaaagtcttccacctcaactgcttcacctgcatggtgtgcaacaagcagctgtccaccggagaagaactctacgtgatcgacgagaacaagtttgtgtgcaaggacgactacttaagctcctctagtctcaaagagggaagcctcaactcagtgtcatcctgtacggaccgcagtttgtctccggacctccaagatccgttacaggacgaccccaaagagaccgacaactcgacctcatcggacaaggagaccgctaacaacgagaatgaggaacagaactccggcaccaaacggcgcggcccgcgcactaccatcaaggccaagcagctggagacgctcaaggcagccttcgcagccacgcccaagcccacgcgccacatccgcgaacagctggcacaagagaccggcctcaacatgagggtcattcaggtgtggtttcagaaccgaaggtccaaagaacgccgcatgaaacagctgagcgctctgggcgcccggagacacgccttcttccggagtccgcggcgcatgcgtcccctgggcggccgcttggacgagtctgagatgttggggtctaccccatatacttattatggagactaccaaagcgactactacgctccgggaggcaactacgatttcttcgcgcacggcccgccgtcgcaggcacagtctccggccgactcaagcttcttggcagcatcgggacctggctcgacgccgcttggcgcgctggaaccaccgctggctgggcctcacggcgcggacaaccctaggttcaccgacatgatctcgcacccggacacgcccagtccggagccaggcttgcccggagcgctgcaccccatgccgggagaggtgttcagcggcgggcccagcccgcccttccccatgagcggcaccagcggctacagcggacccctgtcgcaccccaatcctgagctcaacgaagcggccgtatggtaaggccgaggggccgagttgacccctgccaccaagccccggacgccgcctgggtaagccacaagagtcttctcttgagtttgcacccaccaggcaactcgcatcaccacccctcagagcttcggcacgcgcctgcacagtttctcgggaccaaagtcaatattctggagggtcgagattccaagcacaccctagaagccctccggacccccacccaaccatcacctctttgaattaagagggggaggggatgagacaaggaacggagatcgtggtactacccctccctgcgagccgaggcattgtggaatcctatttctcgctttctctttttaaaaaggggaattc

SEQ ID NO: 14 - protein, 402 aa, Rattus norvegicus; identical to SEQ ID NO: 10

SEQ ID NO: 15 - DNA, 4156 bp, Mus musculus
gagttcagagacagagaaaggcgctgtcagactacgctccacttggggctcgcttgccgcgggtttccgcgcacccacactttctgtggtggtggggacaccctgccccaatccaggcccccattctgcccaccacccccccgccgggatttgagcgccattagctcgcgccagtaccgggatcttcgctcagctcgcctgagtcccgcccgctggattcgaggacccgcttggatgcttcgcctccgagcgccctcggaagatgggccggcagctacaagcctaaagacgtcaagggcttaccagtctccaaggcaaacccggagctctaagcccgctgttcttccgtggaggagatggcttccagcccactgccagggcccaatgatatcctacttgcatcgccatcgagcgccttccagcccgacgcattgagccaaccgcggccgggtcacgccaaccttaaacccaaccaggtgggccaggtgatcctctatggcattcccatcgtgtccttggtgatcgacgggcaggagcgcctgtgcctggcgcagatctccaacactcttctcaagaacttcagctataacgagatccacaaccgccgagtggctctgggcatcacgtgcgtgcagtgcacacctgtccaactggagatcctgcggcgggccggggccatgcccatctcttcgcggcgttgtggcatgatcaccaagcgtgaggccgagcgcctttgtaagtcattcctgggcgaaaatcggccacccaagctgccggacaacttcgccttcgacgtgtcgcacgagtgtgcctggggctgccgcggcagcttcatcccggcgcgctacaacagctctcgcgccaagtgcatcaagtgtagctactgcaacatgtacttctcccccaacaagttcattttccactcccaccgcaccccagacgccaagtacacacagccagacgcggctaacttcaattcctggcgccgccatctcaagctcacagacaagagccctcaggacgagctagtcttcgcctgggaggacgtcaaggccatgttcaacggtggcagccgcaagcgcgcgctacctcaacccagcgcgcacccggcctgtcacccactcagttccgtcaaggctgctgcggtggcggccgcagccgcagtggccgggggcggaggcctgctgggcccgcacctgctgggggctccacccccgccaccgccgccaccacccttggctgagctggcgggcgcacctcacgcccatcacaagcggccgcgcttcgacgacgatgacgattccctccaggaagctgccgtggtggctgcagccagcctctcggcagccgcagccagcctctcggtggccgcagccacaggcggcgccgggccaggcgcaggtggccccgggggtggctgcgtggccggcgtgggcgtgggtgccagtgcgggggctggtgcagcagctggcaccaaaggtccacgcagctacccagtcatcccagtgcccagcaagggttcgttcgggggcgtgctacagaagttcccgggctgcgggggcctcttcccgcatccttacaccttcccggcagcggccgcagccttcggcttgtgccacaagaaggaggacgcggggacagcggccgaggccctggggggagcgggcgcggggagcgcaggtgcggcgcccaaggcagggctgtcgggtctcttctggcccgcgggtcgcaaggacgccttctaccctcccttctgcatgttctggccaccgcggacccccggcgggctgcccgtgcccacctacctacagcccccgccgcagcccccgtctgcgctcggctgcgcgctgggtgatagcccggccctgctgcgtcaggccttcctggacctggccgagccgggcggtgcaggtggcagcgccgaggcagcgccccctccgggccaacctccccccgtggtggccaatggccctggctccggtcctccagctactgggggcactggagcacgcgacacgctcttcgagtcgcccccgggcggcagcggcggggactgcagcgccgggtccacgccacccgcagagcaaggagtgacgtccgggaccgggtctgcgtcctccggagcaggctctgtgggcacccgagtgccggctccccatcacccgcacctcctggaagggcgcaaggcgggcggcggcagctaccaccattccagcgccttccgtccggtgggcggcaaggacgacgcagaaagcctggccaagctgcacggggcgtcggcgggcacaccccactcagccccagcgcatcaccatcaccaccaccatcacccgcaccatcaccaccatcaccctccgcagccgccgtcgccactgctgctgttgcagccccagcccgatgagccggggtcggagcgccaccacccagccccgccacccccgccaccgccgccccctctggccccgcagccgcaccaccgaggccttctgtcccccgagggcaccagctgcagctaccccagtgaggacagctctgaagacgaggaggacgaggaggaagagcaggaggtggacgtggagggccacaagccactcgaaggcgaggaagaggaggacggtcgcgatcctgaagatgaggaggaagaagatgaggagacccgggtccttctaggagactccctggttggcggtggccggttcctccagggccgagggctatcggagaaggggagcggtcgggaccgcacgacgcccgccgtgggtgctttccctctagcgctgaactcctccaggctgctacaagaggatgggaaactgggggactctggaggctcggacctgccggcgcccccgcccccacccctggccccccagaaagcaagcagcagtgggggcagcaggccaggcagccctgtccaccatccatcactggaggaggagccctcgtacaaagataatcagaaacctaaggaaaacaaccaagttattatatctacaaaggatgacaacttctcagataagaacaagggacatggcttcttcatcacagattctgattcttctggagacttttggagagaaagatcaggtgaacatacacaagaaaccaattcacctcattcgctcaaaaaggatgtagaaaacatgggaaaagaggaacttcagaaggttttgtttgagcaaatagatttgcggaggcggctggagcaagaattccaagtgttaaaaggaaatacgtccttcccagtcttcaataactttcaggatcagatgaaaagggaactggcttaccgagaagagatggtgcaacagttacaaatcatcccctatgcagcaagcttgatcaggaaagagaagcttggcgcccatctcagcaaaagctaaaaggcgacagacccactcactcttgttctgtaagatacagcctaccactgagcacttcggacctgcagaaagaagaactgcaaactgaaagggcttgggcaccaaaaccaggttcgagagaagccaaggacaagtgacctcgcgccagcatgggcaaccatgtaaaatagactgtggcggccattcattaggaggggggggaggggggagccaaccagagcggtcaatgcttgtcacatcttttggtggaaccaaagtttgaatatttgtgttttgaaagcatagctgatcccagagggtaggaagtgctgagtggggaaatgtttgtgtggtctctttggcgccatacgattatcctttgctcttccggaagtaattcatagtggaaagaggacagaatggggacttaggtttagacaaacctgctttccataccaagctggacagacacacatggccccttcctctctggtactttgcctgctgtatgaagattgtattcctctggaaatattttacagtttaatattgagtgtaattaagaatataatcatgttatcaaaaatggtatttaactctgttgtagtttctttaacattcatgtggataaaaagtttataataaaaaaactatgacgtaatagatgtgttcatgtagttaagtgcatatatgcttgggggcaactcagaaacgtaatgctttttagagttattttggcataaagtatttgaatataattatttttgaaaacaaaaaaaaaaaaaaaaaaaaaa

SEQ ID NO: 16 - protein, 1008 aa, Mus musculus
MASSPLPGPNDILLASPSSAFQPDALSQPRPGHANLKPNQVGQVILYGIPIVSLVIDGQERLCLAQISNTLLKNFSYNEIHNRRVALGITCVQCTPVQLEILRRAGAMPISSRRCGMITKREAERLCKSFLGENRPPKLPDNFAFDVSHECAWGCRGSFIPARYNSSRAKCIKCSYCNMYFSPNKFIFHSHRTPDAKYTQPDAANFNSWRRHLKLTDKSPQDELVFAWEDVKAMFNGGSRKRALPQPSAHPACHPLSSVKAAAVAAAAAVAGGGGLLGPHLLGAPPPPPPPPPLAELAGAPHAHHKRPRFDDDDDSLQEAAVVAAASLSAAAASLSVAAATGGAGPGAGGPGGGCVAGVGVGASAGAGAAAGTKGPRSYPVIPVPSKGSFGGVLQKFPGCGGLFPHPYTFPAAAAAFGLCHKKEDAGTAAEALGGAGAGSAGAAPKAGLSGLFWPAGRKDAFYPPFCMFWPPRTPGGLPVPTYLQPPPQPPSALGCALGDSPALLRQAFLDLAEPGGAGGSAEAAPPPGQPPPVVANGPGSGPPATGGTGARDTLFESPPGGSGGDCSAGSTPPAEQGVTSGTGSASSGAGSVGTRVPAPHHPHLLEGRKAGGGSYHHSSAFRPVGGKDDAESLAKLHGASAGTPHSAPAHHHHHHHHPHHHHHHPPQPPSPLLLLQPQPDEPGSERHHPAPPPPPPPPPLAPQPHHRGLLSPEGTSCSYPSEDSSEDEEDEEEEQEVDVEGHKPLEGEEEEDGRDPEDEEEEDEETRVLLGDSLVGGGRFLQGRGLSEKGSGRDRTTPAVGAFPLALNSSRLLQEDGKLGDSGGSDLPAPPPPPLAPQKASSSGGSRPGSPVHHPSLEEEPSYKDNQKPKENNQVIISTKDDNFSDKNKGHGFFITDSDSSGDFWRERSGEHTQETNSPHSLKKDVENMGKEELQKVLFEQIDLRRRLEQEFQVLKGNTSFPVFNNFQDQMKRELAYREEMVQQLQIIPYAASLIRKEKLGAHLSKS

SEQ ID NO: 17 - DNA, 3045 bp, Homo sapiens
atggcttccagtccgctgccagggcccaacgacatcctgctggcgtcgccgtcgagcgccttccagcccgacacgctgagccagccgcggccagggcacgccaacctcaaacccaaccaggtgggccaggtgatcctctacggcattcccatcgtgtcgttggtgatcgacgggcaagagcgcctgtgcctggcgcagatctccaacactctgctcaagaacttcagctacaacgagatccacaaccgtcgcgtggcactgggcatcacgtgtgtgcagtgcacgccggtgcaactggagatcctgcggcgtgccggggccatgcccatctcatcgcgccgctgcggcatgatcaccaaacgcgaggccgagcgtctgtgcaagtcgttcctgggcgaaaacaggccgcccaagctgccagacaatttcgccttcgacgtgtcacacgagtgcgcctggggctgccgcggcagcttcattcccgcgcgctacaacagctcgcgcgccaagtgcatcaaatgcagctactgcaacatgtacttctcgcccaacaagttcattttccactcccaccgcacgcccgacgccaagtacactcagccagacgcagccaacttcaactcgtggcgccgtcatctcaagctcaccgacaagagtccccaggacgagctggtcttcgcctgggaggacgtcaaggccatgttcaacggcggcagccgcaagcgcgcactgccccagccgggcgcgcaccccgcctgccacccgctcagctctgtgaaggcggccgccgtggccgccgcggccgcggtggccggaggcgggggtctgctgggcccccacctgctgggtgcgcccccgccgccgccgccgccaccgccgcccttggcagagctggctggtgccccgcacgcccatcacaagcggccgcgcttcgacgacgacgacgactccttgcaggaggccgccgtagtggccgccgccagcctctcggccgcagccgccagcctctctgtggctgctgcttcgggcggcgcggggactggtgggggcggcgctgggggtggctgtgtggccggcgtgggcgtgggcgcgggcgcgggggcgggtgccggggcaggggccaaaggcccgcgcagctacccagtcatcccggtgcccagcaagggctcgttcgggggcgtcctgcagaagttcccgggctgcggcgggctcttcccgcacccctacaccttccctgccgcggccgccgccttcagcttgtgccataagaaagaggatgcgggtgccgccgctgaggccctggggggcgcgggcgcaggcggcgcgggcgcggcgcccaaggccggcttgtccggcctcttctggcccgcgggccgcaaggacgccttctatccgcccttctgcatgttctggccgccgcggacccctggcgggctcccggtgcccacctacctgcagcccccgcctcagccgccctcggcgctaggctgcgcgctaggcgaaagcccggccctgctgcgccaggccttcctggacctggctgagccaggcggtgctgctgggagcgccgaggccgcgcccccgccggggcagcccccgcaggtagtggccaacggcccgggctccggcccacctcctcctgccgggggcgccggctctcgcgacgcgctcttcgagtcgcccccgggcggcagcggcggggactgcagcgcgggctccacgccgcccgcggactctgtggcagctgccggggcaggggccgcggccgccgggtctggccccgcgggctcccgggttccggcgccccaccatccgcaccttctggaggggcgcaaagcgggcggtggcagctaccaccattccagcgccttccggccagtgggcggcaaggacgacgcggagagcctggccaagctgcacggggcgtcggcgggcgcgccccactcggcccagacgcatccccaccaccatcaccaccctcaccaccaccaccaccaccaccaccccccgcagccgccgtcgccgcttctgctgctgcccccgcagcccgacgagccgggttccgagcgccaccacccggccccgccgccgccgccgccgccgcccccgccgccccctctggcccagcacccgcaccaccgaggccttctgtctcccgggggaaccagctgctgctaccccagcgaggacagctccgaggacgaggacgacgaggaagaagagcaggaggtggacgtggagggccacaagccccccgagggcgaggaagaggaggaaggtcgagaccctgacgacgacgaggaagaggacgaggagacggaggtcctactcggcgaccccttagtcgggggcggccggttcctccagggccgagggccgtcggagaaggggagcagccgggaccgcgcgccggccgtcgcgggcgcgttcccgctcggcctgaactcctccaggctgctgcaggaagacgggaaactcggggaccccggctcggacctgcccccgcccccgccgccgcccctggccccccagaaggcgagtggcggcggcagcagcagcccgggcagcccagttcaccatccatcactggaggagcagccctcctacaaagatagtcagaaaactaaggaaaataaccaagtaattgtatctacaaaggatgacaacgttctagataagaacaaggagcatagctttttcatcacagactctgatgcttctggaggagatttttggagagaaagatcaggtgaacatacacaagaaaccaactcacctcattcactgaaaaaggatgtagaaaatatggggaaagaagaacttcagaaggttttatttgaacaaatagatttacggagacgactggaacaagaattccaggtgttaaaaggaaacacatctttcccagtattcaataattttcaggatcagatgaaaagggagctagcctaccgagaagaaatggtgcaacagttacaaattatcccctatgcagcaagtttgatcaggaaagaaaagcttggcgcccatctcagcaaaagctaa

SEQ ID NO: 18 - protein, 1014 aa, Homo sapiens
MASSPLPGPNDILLASPSSAFQPDTLSQPRPGHANLKPNQVGQVILYGIPIVSLVIDGQERLCLAQISNTLLKNFSYNEIHNRRVALGITCVQCTPVQLEILRRAGAMPISSRRCGMITKREAERLCKSFLGENRPPKLPDNFAFDVSHECAWGCRGSFIPARYNSSRAKCIKCSYCNMYFSPNKFIFHSHRTPDAKYTQPDAANFNSWRRHLKLTDKSPQDELVFAWEDVKAMFNGGSRKRALPQPGAHPACHPLSSVKAAAVAAAAAVAGGGGLLGPHLLGAPPPPPPPPPPLAELAGAPHAHHKRPRFDDDDDSLQEAAVVAAASLSAAAASLSVAAASGGAGTGGGGAGGGCVAGVGVGAGAGAGAGAGAKGPRSYPVIPVPSKGSFGGVLQKFPGCGGLFPHPYTFPAAAAAFSLCHKKEDAGAAAEALGGAGAGGAGAAPKAGLSGLFWPAGRKDAFYPPFCMFWPPRTPGGLPVPTYLQPPPQPPSALGCALGESPALLRQAFLDLAEPGGAAGSAEAAPPPGQPPQVVANGPGSGPPPPAGGAGSRDALFESPPGGSGGDCSAGSTPPADSVAAAGAGAAAAGSGPAGSRVPAPHHPHLLEGRKAGGGSYHHSSAFRPVGGKDDAESLAKLHGASAGAPHSAQTHPHHHHHPHHHHHHHHPPQPPSPLLLLPPQPDEPGSERHHPAPPPPPPPPPPPPLAQHPHHRGLLSPGGTSCCYPSEDSSEDEDDEEEEQEVDVEGHKPPEGEEEEEGRDPDDDEEEDEETEVLLGDPLVGGGRFLQGRGPSEKGSSRDRAPAVAGAFPLGLNSSRLLQEDGKLGDPGSDLPPPPPPPLAPQKASGGGSSSPGSPVHHPSLEEQPSYKDSQKTKENNQVIVSTKDDNVLDKNKEHSFFITDSDASGGDFWRERSGEHTQETNSPHSLKKDVENMGKEELQKVLFEQIDLRRRLEQEFQVLKGNTSFPVFNNFQDQMKRELAYREEMVQQLQIIPYAASLIRKEKLGAHLSKS

SEQ ID NO: 19 - DNA, 3123 bp, Mus musculus
gctctctccgacccgcaggcccacgggagcctgagctccgcctccccagggcgcggaagctggcgaagccccagggattcccatttatagcttggtttccactcagctcagtccctccaggactcgggctgagcaagtttcttccattcccttctctcctccctccacccccttctcctcctccttctccttcttttcttcctcctcattcccgcctccccttcaacctcagcagggtgcaggtgtccaactcgaacaagggccccaacttggactcagatgttcccactctcagaccccctgataatgcaggggcgcccgcctgctgcgcggacagctaccctgagcatccgtagccgtccgcacacaaggcgcgggagtttctcaatgggaagaggccgggactctaggaggcggggcgaataggattcctcccgcctagtgggtccctcgcagtcctagggttgcaacccttgagcggtagagaacaccggagactgcggatgagccagatttcggggacataaaatcttccagcccggagagaattgtgtgcagagaggggctccagtccagcgtggtgtgagaggcgtgctatcaagaaagaagttggaggggaaccagtgcaaccctaactctgcgagatcttggggtacacacactcgggatgctggcctccgccctcctcgttttcctttgctgtttcaaaggacatgcaggctcatcgccccatttcctacaacagccagaggacatggtggtgctgttgggggaggaagcccggctgccctgcgctctgggcgcgtacagggggctcgtgcagtggactaaggatgggctggctctagggggcgaaagagaccttccagggtggtcccggtactggatatcggggaattcagccagtggccagcatgacctccacattaagcctgtggaattggaagatgaggcatcgtatgagtgccaggcttcgcaagcaggtctccgatcacgaccagcccaactgcacgtgatggtccccccagaagctccccaggtactaggcggcccctctgtgtctctggttgctggagttcctggaaatctgacctgtcggagtcgtggggattcacgacctgcccctgaactactgtggttccgagatgggatccggctggatgggagcagcttccaccagaccacgctgaaggacaaggccactggaacagtggaaaacaccttattcctgaccccttccagtcatgatgatggtgccaccttgatctgcagagcgcgaagccaggccctgcccacagggagggacacagctgttacactgagccttcagtatcccccaatggtgactctgtctgctgagccccagactgtgcaggagggagagaaggtgactttcctgtgtcaagccactgcccagcctcctgtcactggctacaggtgggcgaaggggggatccccggtgcttggggcacgtgggccaaggttggaggtcgttgcagatgccactttcctgactgagccggtgtcctgcgaggtcagcaacgcggtcggaagcgccaaccgcagcaccgcgctggaagtgttgtatggacccattctgcaggcaaaacctaagtccgtgtccgtggacgtggggaaagatgcctccttcagctgtgtctggcgcgggaacccacttccacggataacctggacccgcatgggtggctctcaggtgctgagctccgggcccacgctgcggcttccgtccgtggcactggaggatgcgggcgactatgtatgcagggctgagccgaggagaacgggtctgggaggcggcaaagcgcaggcgaggctgactgtgaacgcaccccctgtagtgacagccctgcaacctgcaccagcctttctgaggggtcctgctcgcctccagtgtgtggtgtttgcctcccctgccccagactcggtggtttggtcttgggacgagggcttcttggaggcaggctcactgggcaggttcctagtggaagccttcccagccccggaagtggaggggggacagggccctggccttatttctgtgctacacatttccggaacccaggagtccgactttaccaccggcttcaactgcagtgcccgcaaccggctaggagagggacgagtccagatccacttgggccgtagagacttgctgcctactgtccggattgtggctggtgcagcatctgcagccacctctctccttatggtcatcactggagtggtcctctgctgctggcgccatggctctctctctaagcaaaagaacttggtccggatcccgggaagcagcgagggttccagttcacgtggccctgaggaggagacaggcagcagtgaggaccggggtcccattgtgcacaccgaccacagtgatttggttcttgaggaaaaagaggctctggagacaaaggatccaaccaacggttactacagggttcgaggggtcagtgtgagccttagccttggggaagctcctggaggaggcctcttcttgccaccgccctctccgatcggtctcccagggactcctacttactatgacttcaagccacatctggacttagtccctccctgcagactgtacagagcgagggcaggttatcttaccaccccccatccccgtgccttcaccagctacatgaaacccacatcctttggacccccagaattgagctctggaactccccccttcccgtatgctaccttgtctccacccagccaccagcgtctccagactcatgtgtgaatccatctctccaagtgaagggtcttggaatcttctgtttgccatatagtgtgttgtccagatttctggggagtcagaacaagttgatgaccaacccctccaaaactgaacattgaaggagggaaagatcattacaagcatcaggactgttggtgtacactcagttcagccaaagtggattctccaagtgggagcaatatggccgctttcccatgagaaagacattcaagatggtgactaaatgactaaatactttgcagagggacaaagatgggaactagggatatggatggaagtagtagagaagatatatgaccatctgcatcaagagaaaggataacataagacaaatc

SEQ ID NO: 20 - protein, 700 aa, Mus musculus
MLASALLVFLCCFKGHAGSSPHFLQQPEDMVVLLGEEARLPCALGAYRGLVQWTKDGLALGGERDLPGWSRYWISGNSASGQHDLHIKPVELEDEASYECQASQAGLRSRPAQLHVMVPPEAPQVLGGPSVSLVAGVPGNLTCRSRGDSRPAPELLWFRDGIRLDGSSFHQTTLKDKATGTVENTLFLTPSSHDDGATLICRARSQALPTGRDTAVTLSLQYPPMVTLSAEPQTVQEGEKVTFLCQATAQPPVTGYRWAKGGSPVLGARGPRLEVVADATFLTEPVSCEVSNAVGSANRSTALEVLYGPILQAKPKSVSVDVGKDASFSCVWRGNPLPRITWTRMGGSQVLSSGPTLRLPSVALEDAGDYVCRAEPRRTGLGGGKAQARLTVNAPPVVTALQPAPAFLRGPARLQCVVFASPAPDSVVWSWDEGFLEAGSLGRFLVEAFPAPEVEGGQGPGLISVLHISGTQESDFTTGFNCSARNRLGEGRVQIHLGRRDLLPTVRIVAGAASAATSLLMVITGVVLCCWRHGSLSKQKNLVRIPGSSEGSSSRGPEEETGSSEDRGPIVHTDHSDLVLEEKEALETKDPTNGYYRVRGVSVSLSLGEAPGGGLFLPPPSPIGLPGTPTYYDFKPHLDLVPPCRLYRARAGYLTTPHPRAFTSYMKPTSFGPPELSSGTPPFPYATLSPPSHQRLQTHV

SEQ ID NO: 21 - DNA, 2847 bp, Mus musculus
ggtccggaattcccgggatgagaggggctccagtccagcgtggtgtgagaggcgtgctatcaagaaagaagttggaggggaaccagtgcaaccctaactctgcgagatcttggggtacacacactcgggatgctggcctccgccctcctcgttttcctttgctgtttcaaaggacatgcaggctcatcgccccatttcctacaacagccagaggacatggtggtgctgttgggggaggaagcccggctgccctgcgctctgggcgcgtacagggggctcgtgcagtggactaaggatgggctggctctagggggcgaaagagaccttccagggtggtcccggtactggatatcggggaattcagccagtggccagcatgacctccacattaagcctgtggaattggaagatgaggcatcgtatgagtgccaggcttcgcaagcaggtctccgatcacgaccagcccaactgcacgtgatggtccccccagaagctccccaggtactaggcggcccctctgtgtctctggttgctggagttcctggaaatctgacctgtcggagtcgtggggattcacgacctgcccctgaactactgtggttccgagatgggatccggctggatgggagcagcttccaccagaccacgctgaaggacaaggccactggaacagtggaaaacaccttattcctgaccccttccagtcatgatgatggtgccaccttgatctgcagagcgcgaagccaggccctgcccacagggagggacacagctgttacactgagccttcagtatcccccaatggtgactctgtctgctgagccccagactgtgcaggagggagagaaggtgactttcctgtgtcaagccactgcccagcctcctgtcactggctacaggtgggcgaaggggggatccccggtgcttggggcacgtgggccaaggttggaggtcgttgcagatgccactttcctgactgagccggtgtcctgcgaggtcagcaacgcggtcggaagcgccaaccgcagcaccgcgctggaagtgttgtatggacccattctgcaggcaaaacctaagtccgtgtccgtggacgtggggaaagatgcctccttcagctgtgtctggcgcgggaacccacttccacggataacctggacccgcatgggtggctctcaggtgctgagctccgggcccacgctgcggcttccgtccgtggcactggaggatgcgggcgactatgtatgcagggctgagccgaggagaacgggtctgggaggcggcaaagcgcaggcgaggctgactgtgaacgcaccccctgtagtgacagccctgcaacctgcaccagcctttctgaggggtcctgctcgcctccagtgtgtggtgtttgcctcccctgccccagactcggtggtttggtcttgggacgagggcttcttggaggcaggctcactgggcaggttcctagtggaagccttcccagccccggaagtggaggggggacagggccctggccttatttctgtgctacacatttccggaacccaggagtccgactttaccaccggcttcaactgcagtgcccgcaaccggctaggagagggacgagtccagatccacttgggccgtagagacttgctgcctactgtccggattgtggctggtgcagcatctgcagccacctctctccttatggtcatcactggagtggtcctctgctgctggcgccatggctctctctctaagcaaaagaacttggtccggatcccaggaagcagcgagggttccagttcacgtggccctgaggaggagacaggcagcagtgaggaccggggtcccattgtgcacaccgaccacagtgatttggttcttgaggaaaaagaggctctggagacaaaggatccaaccaacggttactacagggttcgaggggtcagtgtgagccttagccttggggaagctcctggaggaggcctcttcttgccaccgccctctccgatcggtctcccagggactcctacttactatgacttcaagccacatctggacttagtccctccctgcagactgtacagagcgagggcaggttatcttaccaccccccatccccgtgccttcaccagctacatgaaacccacatcctttggacccccagaattgagctctggaactccccccttcccgtatgctaccttgtctccacccagccaccagcgtctccagactcatgtgtgaatccatctctccaagtgaagggtcttggaatcttctgtttgccatatagtgtgttgtccagatttctggggagtcagaacaagttgatgaccaacccctccaaaactgaacattgaaggagggaaagatcattacaagcatcaggactgttggtgtacactcagttcagccaaagtggattctccaagtgggagcaatatggccgctttcccatgagaaagacattcaagatggtgactaaatgactaaatactttgcagagggacaaagatgggaactagggatatggatggaagtagtagagaagatatatgaccatctgcatcaagagaaaggataacataagacaaatcaagatgaaagaaataatccacacccccccccccaccgcgtcctggccaataagtatagcctacatggctgttcattatctgggaaccaaaatggccactatcttgactccttccttaaagatacagaaagaattgaatccaaggaatggggtagggtggaaatagaagaaatgaaggggactcttgggctaagaatacttatgtttaataataaaagggggaggcaaagaaaaaaaaaaaaaaaaaaaaaaaaaaaaaaaaa

SEQ ID NO: 22 - protein, 700 aa, Mus musculus; identical to SEQ ID NO: 20

SEQ ID NO: 23 - DNA, 2153 bp, Homo sapiens
ccgcggaactggcaggcgtttcagagcgtcagaggctgcggatgagcagacttggaggactccaggccagagactaggctgggcgaagagtcgagcgtgaagggggctccgggccagggtgacaggaggcgtgcttgagaggaagaagttgacgggaaggccagtgcgacggcaaatctcgtgaaccttgggggacgaatgctcaggatgcgggtccccgccctcctcgtcctcctcttctgcttcagagggagagcaggcccgtcgccccatttcctgcaacagccagaggacctggtggtgctgctgggggaggaagcccggctgccgtgtgctctgggcgcctactgggggctagttcagtggactaagagtgggctggccctagggggccaaagggacctaccagggtggtcccggtactggatatcagggaatgcagccaatggccagcatgacctccacattaggcccgtggagctagaggatgaagcatcatatgaatgtcaggctacacaagcaggcctccgctccagaccagcccaactgcacgtgctggtccccccagaagccccccaggtgctgggcggcccctctgtgtctctggttgctggagttcctgcgaacctgacatgtcggagccgtggggatgcccgccctacccctgaattgctgtggttccgagatggggtcctgttggatggagccaccttccatcagaccctgctgaaggaagggacccctgggtcagtggagagcaccttaaccctgacccctttcagccatgatgatggagccacctttgtctgccgggcccggagccaggccctgcccacaggaagagacacagctatcacactgagcctgcagtaccccccagaggtgactctgtctgcttcgccacacactgtgcaggagggagagaaggtcattttcctgtgccaggccacagcccagcctcctgtcacaggctacaggtgggcaaaagggggctctccggtgctcggggcccgcgggccaaggttagaggtcgtggcagacgcctcgttcctgactgagcccgtgtcctgcgaggtcagcaacgccgtgggtagcgccaaccgcagtactgcgctggatgtgctgtttgggccgattctgcaggcaaagccggagcccgtgtccgtggacgtgggggaagacgcttccttcagctgcgcctggcgcgggaacccgcttccacgggtaacctggacccgccgcggtggcgcgcaggtgctgggctctggagccacactgcgtcttccgtcggtggggcccgaggacgcaggcgactatgtgtgcagagctgaggctgggctatcgggcctgcggggcggcgccgcggaggctcggctgactgtgaacgctcccccagtagtgaccgccctgcactctgcgcctgccttcctgaggggccctgctcgcctccagtgtctggttttcgcctctcccgccccagatgccgtggtctggtcttgggatgagggcttcctggaggcggggtcgcagggccggttcctggtggagacattccctgccccagagagccgcgggggactgggtccgggcctgatctctgtgctacacatttcggggacccaggagtctgactttagcaggagctttaactgcagtgcccggaaccggctgggcgagggaggtgcccaggccagcctgggccgtagagacttgctgcccactgtgcggatagtggccggagtggccgctgccaccacaactctccttatggtcatcactggggtggccctctgctgctggcgccacagcaaggcctcagcctctttctccgagcaaaagaacctgatgcgaatccctggcagcagcgacggctccagttcacgaggtcctgaagaagaggagacaggcagccgcgaggaccggggccccattgtgcacactgaccacagtgatctggttctggaggaggaagggactctggagaccaaggacccaaccaacggttactacaaggtccgaggagtcagtccacccgcgtctccagactcacgtgtgacatctttccaatggaagagtcctgggatctccaacttgccataatggattgttctgatttctgaggagccaggacaagttggcgaccttactcctcc

SEQ ID NO: 24 - protein, 633 aa, Homo sapiens
MLRMRVPALLVLLFCFRGRAGPSPHFLQQPEDLVVLLGEEARLPCALGAYWGLVQWTKSGLALGGQRDLPGWSRYWISGNAANGQHDLHIRPVELEDEASYECQATQAGLRSRPAQLHVLVPPEAPQVLGGPSVSLVAGVPANLTCRSRGDARPTPELLWFRDGVLLDGATFHQTLLKEGTPGSVESTLTLTPFSHDDGATFVCRARSQALPTGRDTAITLSLQYPPEVTLSASPHTVQEGEKVIFLCQATAQPPVTGYRWAKGGSPVLGARGPRLEVVADASFLTEPVSCEVSNAVGSANRSTALDVLFGPILQAKPEPVSVDVGEDASFSCAWRGNPLPRVTWTRRGGAQVLGSGATLRLPSVGPEDAGDYVCRAEAGLSGLRGGAAEARLTVNAPPVVTALHSAPAFLRGPARLQCLVFASPAPDAVVWSWDEGFLEAGSQGRFLVETFPAPESRGGLGPGLISVLHISGTQESDFSRSFNCSARNRLGEGGAQASLGRRDLLPTVRIVAGVAAATTTLLMVITGVALCCWRHSKASASFSEQKNLMRIPGSSDGSSSRGPEEEETGSREDRGPIVHTDHSDLVLEEEGTLETKDPTNGYYKVRGVSPPASPDSRVTSFQWKSPGISNLP

SEQ ID NO: 25 - DNA, 2979 bp, Homo sapiens
ccgcggaactggcaggcgtttcagagcgtcagaggctgcggatgagcagacttggaggactccaggccagagactaggctgggcgaagagtcgagcgtgaagggggctccgggccagggtgacaggaggcgtgcttgagaggaagaagttgacgggaaggccagtgcgacggcaaatctcgtgaaccttgggggacgaatgctcaggatgcgggtccccgccctcctcgtcctcctcttctgcttcagagggagagcaggcccgtcgccccatttcctgcaacagccagaggacctggtggtgctgctgggggaggaagcccggctgccgtgtgctctgggcgcctactgggggctagttcagtggactaagagtgggctggccctagggggccaaagggacctaccagggtggtcccggtactggatatcagggaatgcagccaatggccagcatgacctccacattaggcccgtggagctagaggatgaagcatcatatgaatgtcaggctacacaagcaggcctccgctccagaccagcccaactgcacgtgctggtccccccagaagccccccaggtgctgggcggcccctctgtgtctctggttgctggagttcctgcgaacctgacatgtcggagccgtggggatgcccgccctacccctgaattgctgtggttccgagatggggtcctgttggatggagccaccttccatcagaccctgctgaaggaagggacccctgggtcagtggagagcaccttaaccctgacccctttcagccatgatgatggagccacctttgtctgccgggcccggagccaggccctgcccacaggaagagacacagctatcacactgagcctgcagtaccccccagaggtgactctgtctgcttcgccacacactgtgcaggagggagagaaggtcattttcctgtgccaggccacagcccagcctcctgtcacaggctacaggtgggcaaaagggggctctccggtgctcggggcccgcgggccaaggttagaggtcgtggcagacgcctcgttcctgactgagcccgtgtcctgcgaggtcagcaacgccgtgggtagcgccaaccgcagtactgcgctggatgtgctgtttgggccgattctgcaggcaaagccggagcccgtgtccgtggacgtgggggaagacgcttccttcagctgcgcctggcgcgggaacccgcttccacgggtaacctggacccgccgcggtggcgcgcaggtgctgggctctggagccacactgcgtcttccgtcggtggggcccgaggacgcaggcgactatgtgtgcagagctgaggctgggctatcgggcctgcggggcggcgccgcggaggctcggctgactgtgaacgctcccccagtagtgaccgccctgcactctgcgcctgccttcctgaggggccctgctcgcctccagtgtctggttttcgcctctcccgccccagatgccgtggtctggtcttgggatgagggcttcctggaggcggggtcgcagggccggttcctggtggagacattccctgccccagagagccgcgggggactgggtccgggcctgatctctgtgctacacatttcggggacccaggagtctgactttagcaggagctttaactgcagtgcccggaaccggctgggcgagggaggtgcccaggccagcctgggccgtagagacttgctgcccactgtgcggatagtggccggagtggccgctgccaccacaactctccttatggtcatcactggggtggccctctgctgctggcgccacagcaaggcctcagcctctttctccgagcaaaagaacctgatgcgaatccctggcagcagcgacggctccagttcacgaggtcctgaagaagaggagacaggcagccgcgaggaccggggccccattgtgcacactgaccacagtgatctggttctggaggaggaagggactctggagaccaaggacccaaccaacggttactacaaggtccgaggagtcagtgtgagcctgagccttggcgaagcccctggaggaggtctcttcctgccaccaccctccccccttgggcccccagggacccctaccttctatgacttcaacccacacctgggcatggtccccccctgcagactttacagagccagggcaggctatctcaccacaccccaccctcgagctttcaccagctacatcaaacccacatcctttgggcccccagatctggcccccgggactccccccttcccatatgctgccttccccacacctagccacccgcgtctccagactcacgtgtgacatctttccaatggaagagtcctgggatctccaacttgccataatggattgttctgatttctgaggagccaggacaagttggcgaccttactcctccaaaactgaacacaaggggagggaaagatcattacatttgtcaggagcatttgtatacagtcagctcagccaaaggagatgccccaagtgggagcaacatggccacccaatatgcccacctattccccggtgtaaaagagattcaagatggcaggtaggccctttgaggagagatggggacagggcagtgggtgttgggagtttggggccgggatggaagttgtttctagccactgaaagaagatatttcaagatgaccatctgcattgagaggaaaggtagcataggatagatgaagatgaagagcataccaggccccaccctggctctccctgaggggaactttgctcggccaatggaaatgcagccaagatggccatatactccctaggaacccaagatggccaccatcttgattttactttccttaaagactcagaaagacttggacccaaggagtggggatacagtgagaattaccactgttggggcaaaatattgggataaaaatatttatgtttaataataaaaaaaagtcaaagaggcaaaaaaaaaaaa

SEQ ID NO: 26 - protein, 708 aa, Homo sapiens
MLRMRVPALLVLLFCFRGRAGPSPHFLQQPEDLVVLLGEEARLPCALGAYWGLVQWTKSGLALGGQRDLPGWSRYWISGNAANGQHDLHIRPVELEDEASYECQATQAGLRSRPAQLHVLVPPEAPQVLGGPSVSLVAGVPANLTCRSRGDARPTPELLWFRDGVLLDGATFHQTLLKEGTPGSVESTLTLTPFSHDDGATFVCRARSQALPTGRDTAITLSLQYPPEVTLSASPHTVQEGEKVIFLCQATAQPPVTGYRWAKGGSPVLGARGPRLEVVADASFLTEPVSCEVSNAVGSANRSTALDVLFGPILQAKPEPVSVDVGEDASFSCAWRGNPLPRVTWTRRGGAQVLGSGATLRLPSVGPEDAGDYVCRAEAGLSGLRGGAAEARLTVNAPPVVTALHSAPAFLRGPARLQCLVFASPAPDAVVWSWDEGFLEAGSQGRFLVETFPAPESRGGLGPGLISVLHISGTQESDFSRSFNCSARNRLGEGGAQASLGRRDLLPTVRIVAGVAAATTTLLMVITGVALCCWRHSKASASFSEQKNLMRIPGSSDGSSSRGPEEEETGSREDRGPIVHTDHSDLVLEEEGTLETKDPTNGYYKVRGVSVSLSLGEAPGGGLFLPPPSPLGPPGTPTFYDFNPHLGMVPPCRLYRARAGYLTTPHPRAFTSYIKPTSFGPPDLAPGTPPFPYAAFPTPSHPRLQTHV

SEQ ID NO: 27 - DNA, 2973 bp, Homo sapiens
ccgcggaactggcaggcgtttcagagcgtcagaggctgcggatgagcagacttggaggactccaggccagagactaggctgggcgaagagtcgagcgtgaagggggctccgggccagggtgacaggaggcgtgcttgagaggaagaagttgacgggaaggccagtgcgacggcaaatctcgtgaaccttgggggacgaatgctcaggatgcgggtccccgccctcctcgtcctcctcttctgcttcagagggagagcaggcccgtcgccccatttcctgcaacagccagaggacctggtggtgctgctgggggaggaagcccggctgccgtgtgctctgggcgcctactgggggctagttcagtggactaagagtgggctggccctagggggccaaagggacctaccagggtggtcccggtactggatatcagggaatgcagccaatggccagcatgacctccacattaggcccgtggagctagaggatgaagcatcatatgaatgtcaggctacacaagcaggcctccgctccagaccagcccaactgcacgtgctggtccccccagaagccccccaggtgctgggcggcccctctgtgtctctggttgctggagttcctgcgaacctgacatgtcggagccgtggggatgcccgccctacccctgaattgctgtggttccgagatggggtcctgttggatggagccacctttcatcagaccctgctgaaggaagggacccctgggtcagtggagagcaccttaaccctgacccctttcagccatgatgatggagccacctttgtctgccgggcccggagccaggccctgcccacaggaagagacacagctatcacactgagcctgcagtaccccccagaggtgactctgtctgcttcgccacacactgtgcaggagggagagaaggtcattttcctgtgccaggccacagcccagcctcctgtcacaggctacaggtgggcaaaagggggctctccggtgctcggggcccgcgggccaaggttagaggtcgtggcagacgcctcgttcctgactgagcccgtgtcctgcgaggtcagcaacgccgtgggtagcgccaaccgcagtactgcgctggatgtgctgtttgggccgattctgcaggcaaagccggagcccgtgtccgtggacgtgggggaagacgcttccttcagctgcgcctggcgcgggaacccgcttccacgggtaacctggacccgccgcggtggcgcgcaggtgctgggctctggagccacactgcgtcttccgtcggtggggcccgaggacgcaggcgactatgtgtgcagagctgaggctgggctatcgggcctgcggggcggcgccgcggaggctcggctgactgtgaacgctcccccagtagtgaccgccctgcactctgcgcctgccttcctgaggggccctgctcgcctccagtgtctggttttcgcctctcccgccccagatgccgtggtctggtcttgggatgagggcttcctggaggcggggtcgcagggccggttcctggtggagacattccctgccccagagagccgcgggggactgggtccgggcctgatctctgtgctacacatttcggggacccaggagtctgactttagcaggagctttaactgcagtgcccggaaccggctgggcgagggaggtgcccaggccagcctgggccgtagagacttgctgcccactgtgcggatagtggccggagtggccgctgccaccacaactctccttatggtcatcactggggtggccctctgctgctggcgccacagcaaggcctcagcctctttctccgagcaaaagaacctgatgcgaatccctggcagcagcgacggctccagttcacgaggtcctgaagaagaggagacaggcagccgcgaggaccggggccccattgtgcacactgaccacagtgatctggttctggaggaggaagggactctggagaccaaggacccaaccaacggttactacaaggtccgaggagtcagtgtgagcctgagccttggcgaagcccctggaggaggtctcttcctgccaccaccctccccccttgggcccccagggacccctaccttctatgacttcaacccacacctgggcatggtccccccctgcagactttacagagccagggcaggctatctcaccacaccccaccctcgagctttcaccagctacatcaaacccacatcctttgggcccccagatctggcccccgggactccccccttcccatatgctgccttccccacacctagccacccgcgtctccagactcacgtgtgacatctttccaatggaagagtcctgggatctccaacttgccataatggattgttctgatttctgaggagccaggacaagttggcgaccttactcctccaaaactgaacacaaggggagggaaagatcattacatttgtcaggagcatttgtatacagtcagctcagccaaaggagatgccccaagtgggagcaacatggccacccaatatgcccacctattccccggtgtaaaagagattcaagatggcaggtaggccctttgaggagagatggggacagggcagtgggtgttgggagtttggggccgggatggaagttgtttctagccactgaaagaagatatttcaagatgaccatctgcattgagaggaaaggtagcataggatagatgaagatgaagagcataccaggccccaccctggctctccctgaggggaactttgctcggccaatggaaatgcagccaagatggccatatactccctaggaacccaaaatggccaccatcttgattttactttccttaaagactcagaaagacttggacccaaggagtggggatacagtgagaattaccactgttggggcaaaatattgggataaaaatatttatgtttaataataaaaaaaagtcaaagagaaaaaaaa

SEQ ID NO: 28 - protein, 708 aa, Homo sapiens; identical to SEQ ID NO: 26

SEQ ID NO: 29 - DNA, 1969 bp, Homo sapiens
accttgggggacgaatgctcaggatgcgggtccccgccctcctcgtcctcctcttctgcttcagagggagagcaggcccgtcgccccatttcctgcaacagccagaggacctggtggtgctgctgggggaggaagcccggctgccgtgtgctctgggcgcctactgggggctagttcagtggactaagagtgggctggccctagggggccaaagggacctaccagggtggtcccggtactggatatcagggaatgcagccaatggccagcatgacctccacattaggcccgtggagctagaggatgaagcatcatatgaatgtcaggctacacaagcaggcctccgctccagaccagcccaactgcacgtgctggtccccccagaagccccccaggtgctgggcggcccctctgtgtctctggttgctggagttcctgcgaacctgacatgtcggagccgtggggatgcccgccctacccctgaattgctgtggttccgagatggggtcctgttggatggagccaccttccatcagaccctgctgaaggaagggacccctgggtcagtggagagcaccttaaccctgacccctttcagccatgatgatggagccacccttgtctgccgggcccggagccaggccctgcccacaggaagagacacagctatcacactgagcctgcagtaccccccagaggtgactctgtctgcttcgccacacactgtgcaggagggagagaaggtcattttcctgtgccaggccacagcccagcctcctgtcacaggctacaggtgggcaaaagggggctctccggtgctcggggcccgcgggccaaggttagaggtcgtggcagacgcctcgttcctgactgagcccgtgtcctgcgaggtcagcaacgccgtgggtagcgccaaccgcagtactgcgctggatgtgctgtttgggccgattctgcaggcaaagccggagcccgtgtccgtggacgtgggggaagacgcttccttcagctgcgcctggcgcgggaatccgcttccacgggtaacctggacccgccgcggtggcgcgcaggtgctgggctctggagccacactgcgtcttccgtcggtggggcccgaggacgcagacgactatgtgtgcagagctgaggctgggctatcgggcctgcggggcggcgccgcggaggctcggctgactgtgaacgctcccccagtagtgaccgccctgcactctgcgcctgccttcctgaggggccctgctcgcctccagtgtctggttttcgcctctcccgccccagatgccgtggtctggtcttgggatgagggcttcctggaggcggggtcgcagggtcggttcctggtggagacattccctgccccagagagccgcgggggactgggtccgggcctgatctctgtgctacacatttcggggacccaggagtctgactttagcaggagctttaactgcagtgcccggaaccggctgggcgagggaggtgcccaggccagcctgggccgtagagacttgctgcccactgtgcggatagtggccggagtggtcgctgccaccacaactctccttatggtcatcactggggtggccctctgctgctggcgccacagcaaggcctcagcctctttctccgagcaaaagaacctgatgcgaatccctggcagcagcgacggctccagttcacgaggtcctgaagaagaggagacaggcagccgcgaggaccggggccccattgtgcacactgaccacagtgatctggttctggaggaggaagggactctggagaccaaggacccaaccaacggttactacaaggtccgaggagtcagtccacccgcgtctccagactcacgtgtgacatctttccaatggaagagtcctgggatctccaacttgccataatggattgttctgatttctgaggagccaggacaagttggcgaccttactcctcc

SEQ ID NO: 30 - protein, 633 aa, Homo sapiens
MLRMRVPALLVLLFCFRGRAGPSPHFLQQPEDLVVLLGEEARLPCALGAYWGLVQWTKSGLALGGQRDLPGWSRYWISGNAANGQHDLHIRPVELEDEASYECQATQAGLRSRPAQLHVLVPPEAPQVLGGPSVSLVAGVPANLTCRSRGDARPTPELLWFRDGVLLDGATFHQTLLKEGTPGSVESTLTLTPFSHDDGATLVCRARSQALPTGRDTAITLSLQYPPEVTLSASPHTVQEGEKVIFLCQATAQPPVTGYRWAKGGSPVLGARGPRLEVVADASFLTEPVSCEVSNAVGSANRSTALDVLFGPILQAKPEPVSVDVGEDASFSCAWRGNPLPRVTWTRRGGAQVLGSGATLRLPSVGPEDADDYVCRAEAGLSGLRGGAAEARLTVNAPPVVTALHSAPAFLRGPARLQCLVFASPAPDAVVWSWDEGFLEAGSQGRFLVETFPAPESRGGLGPGLISVLHISGTQESDFSRSFNCSARNRLGEGGAQASLGRRDLLPTVRIVAGVVAATTTLLMVITGVALCCWRHSKASASFSEQKNLMRIPGSSDGSSSRGPEEEETGSREDRGPIVHTDHSDLVLEEEGTLETKDPTNGYYKVRGVSPPASPDSRVTSFQWKSPGISNLP

SEQ ID NO: 31 - DNA, 2003 bp, Homo sapiens
ccgcggaactggcaggcgtttcagagcgtcagaggctgcggatgagcagacttggaggactccaggccagagactaggctgggcgaagagtcgagcgtgaagggggctccgggccagggtgacaggaggcgtgcttgagaggaagaagttgacgggaaggccagtgcgacggcaaatctcgtgaaccttgggggacgaatgctcaggatgcgggtccccgccctcctcgtcctcctcttctgcttcagagggagagcagggtggtcccggtactggatatcagggaatgcagccaatggccagcatgacctccacattaggcccgtggagctagaggatgaagcatcatatgaatgtcaggctacacaagcaggcctccgctccagaccagcccaactgcacgtgctggtccccccagaagccccccaggtgctgggcggcccctctgtgtctctggttgctggagttcctgcgaacctgacatgtcggagccgtggggatgcccgccctacccctgaattgctgtggttccgagatggggtcctgttggatggagccaccttccatcagaccctgctgaaggaagggacccctgggtcagtggagagcaccttaaccctgacccctttcagccatgatgatggagccacctttgtctgccgggcccggagccaggccctgcccacaggaagagacacagctatcacactgagcctgcagtaccccccagaggtgactctgtctgcttcgccacacactgtgcaggagggagagaaggtcattttcctgtgccaggccacagcccagcctcctgtcacaggctacaggtgggcaaaagggggctctccggtgctcggggcccgcgggccaaggttagaggtcgtggcagacgcctcgttcctgactgagcccgtgtcctgcgaggtcagcaacgccgtgggtagcgccaaccgcagtactgcgctggatgtgctgtttgggccgattctgcaggcaaagccggagcccgtgtccgtggacgtgggggaagacgcttccttcagctgcgcctggcgcgggaacccgcttccacgggtaacctggacccgccgcggtggcgcgcaggtgctgggctctggagccacactgcgtcttccgtcggtggggcccgaggacgcaggcgactatgtgtgcagagctgaggctgggctatcgggcctgcggggcggcgccgcggaggctcggctgactgtgaacgctcccccagtagtgaccgccctgcactctgcgcctgccttcctgaggggccctgctcgcctccagtgtctggttttcgcctctcccgccccagatgccgtggtctggtcttgggatgagggcttcctggaggcggggtcgcagggccggttcctggtggagacattccctgccccagagagccgcgggggactgggtccgggcctgatctctgtgctacacatttcggggacccaggagtctgactttagcaggagctttaactgcagtgcccggaaccggctgggcgagggaggtgcccaggccagcctgggccgtagagacttgctgcccactgtgcggatagtggccggagtggccgctgccaccacaactctccttatggtcatcactggggtggccctctgctgctggcgccacagcaaggcctcagcctctttctccgagcaaaagaacctgatgcgaatccctggcagcagcgacggctccagttcacgaggtcctgaagaagaggagacaggcagccgcgaggaccggggccccattgtgcacactgaccacagtgatctggttctggaggaggaagggactctggagaccaaggacccaaccaacggttactacaaggtccgaggagtcagtccacccgcgtctccagactcacgtgtgacatctttccaatggaagagtcctgggatctccaacttgccataatggattgttctgatttctgaggagccaggacaagttggcgaccttactcctcc

SEQ ID NO: 32 - protein, 583 aa, Homo sapiens
MLRMRVPALLVLLFCFRGRAGWSRYWISGNAANGQHDLHIRPVELEDEASYECQATQAGLRSRPAQLHVLVPPEAPQVLGGPSVSLVAGVPANLTCRSRGDARPTPELLWFRDGVLLDGATFHQTLLKEGTPGSVESTLTLTPFSHDDGATFVCRARSQALPTGRDTAITLSLQYPPEVTLSASPHTVQEGEKVIFLCQATAQPPVTGYRWAKGGSPVLGARGPRLEVVADASFLTEPVSCEVSNAVGSANRSTALDVLFGPILQAKPEPVSVDVGEDASFSCAWRGNPLPRVTWTRRGGAQVLGSGATLRLPSVGPEDAGDYVCRAEAGLSGLRGGAAEARLTVNAPPVVTALHSAPAFLRGPARLQCLVFASPAPDAVVWSWDEGFLEAGSQGRFLVETFPAPESRGGLGPGLISVLHISGTQESDFSRSFNCSARNRLGEGGAQASLGRRDLLPTVRIVAGVAAATTTLLMVITGVALCCWRHSKASASFSEQKNLMRIPGSSDGSSSRGPEEEETGSREDRGPIVHTDHSDLVLEEEGTLETKDPTNGYYKVRGVSPPASPDSRVTSFQWKSPGISNLP

SEQ ID NO: 33 - DNA, 1819 bp, Homo sapiens
accttgggggacgaatgctctggatgcgggtccccgccctcctcgtcctcctcttctgcttcagagggagagcagggtggtcccggtactggatatcagggaatgcagccaatggccagcatgacctccacattaggcccgtggagctagaggatgaagcatcatatgaatgtcaggctacacaagcaggcctccgctccagaccagcccaactgcacgtgctggtccccccagaagccccccaggtgctgggcggcccctctgtgtctctggttgctggagttcctgcgaacctgacatgtcggagccgtggggatgcccgccctacccctgaattgctgtggttccgagatggggtcctgttggatggagccaccttccatcagaccctgctgaaggaagggacccctgggtcagtggagagcaccttaaccctgacccctttcagccatgatgatggagccacctttgtctgccgggcccggagccaggccctgcccacaggaagagacacagctatcacactgagcctgcagtaccccccagaggtgactctgtctgcttcgccacacactgtgcaggagggagagaaggtcattttcctgtgccaggccacagcccagcctcctgtcacaggctacaggtgggcaaaagggggctctccggtgctcggggcccgcgggccaaggttagaggtcgtggcagacgcctcgttcctgactgagcccgtgtcctgcgaggtcagcaacgccgtgggtagcgccaaccgcagtactgcgctggatgtgctgtttgggccgattctgcaggcaaagccggagcccgtgtccgtggacgtgggggaagacgcttccttcagctgcgcctggcgcgggaatccgcttccacgggtaacctggacccgccgcggtggcgcgcaggtgctgggctctggagccacactgcgtcttccgtcggtggggcccgaggacgcagacgactatgtgtgcagagctgaggctgggctatcgggtctgcggggcggcgccgtggaggctcggctgactgtggacgctcccccagtagtgaccgccctgcactctgcgcctgccttcctgaggggccctgctcgcctccagtgtctggttttcgcctctcccgccccagatgccgtggtctggtcttgggatgagggcttcctggaggcggggtcgcagggtcggttcctggtggagacattccctgccccagagagccgcgggggactgggtccgggcctgatctctgtgctacacatttcggggacccaggagtctgactttagcaggagctttaactgcagtgcccggaaccggctgggcgagggaggtgcccaggccagcctgggccgtagagacttgctgcccactgtgcggatagtggccggagtggtcgctgccaccacaactctccttatggtcatcactggggtggccctctgctgctggcgccacagcaaggcctcagcctctttctccgagcaaaagaacctgatgcgaatccctggcagcagcgacggctccagttcacgaggtcctgaagaagaggagacaggcagccgcgaggaccggggccccattgtgcacactgaccacagtgatctggttctggaggaggaagggactctggagaccaaggacccaaccaacggttactacaaggtccgaggagtcagtccacccgcgtctccagactcacgtgtgacatctttccaatggaagagtcctgggatctccaacttgccataatggattgttctgatttctgaggagccaggacaagttggcgaccttactcctcc

SEQ ID NO: 34 - protein, 583 aa, Homo sapiens [sequence omitted]

SEQ ID NO: 35 - DNA, 2959 bp, Homo sapiens
atgcacttgagtgcaccttgagtctccagcctctcaaggaaccgggagatcaggccatcagcgtctcagccagcaaaggcctgaaccaccagtcccttataaccctgcgtttcagagcgtcagaggcgtgcttgagaggaagaagttgacgggaaggccagtgcgacggcaaatctcgtgaaccttgggggacgaatgctcaggatgcgggtccccgccctcctcgtcctcctcttctgcttcagagggagagcaggcccgtcgccccatttcctgcaacagccagaggacctggtggtgctgctgggggaggaagcccggctgccgtgtgctctgggcgcctactgggggctagttcagtggactaagagtgggctggccctagggggccaaagggacctaccagggtggtcccggtactggatatcagggaatgcagccaatggccagcatgacctccacattaggcccgtggagctagaggatgaagcatcatatgaatgtcaggctacacaagcaggcctccgctccagaccagcccaactgcacgtgctggtccccccagaagccccccaggtgctgggcggcccctctgtgtctctggttgctggagttcctgcgaacctgacatgtcggagccgtggggatgcccgccctacccctgaattgctgtggttccgagatggggtcctgttggatggagccaccttccatcagaccctgctgaaggaagggacccctgggtcagtggagagcaccttaaccctgcccacaggaagagacacagctatcacactgagcctgcagtaccccccagaggtgactctgtctgcttcgccacacactgtgcaggagggagagaaggtcattttcctgtgccaggccacagcccagcctcctgtcacaggctacaggtgggcaaaagggggctctccggtgctcggggcccgcgggccaaggttagaggtcgtggcagacgcctcgttcctgactgagcccgtgtcctgcgaggtcagcaacgccgtgggtagcgccaaccgcagtactgcgctggatgtgctgtttgggccgattctgcaggcaaagccggagcccgtgtccgtggacgtgggggaagacgcttccttcagctgcgcctggcgcgggaacccgcttccacgggtaacctggacccgccgcggtggcgcgcaggtgctgggctctggagccacactgcgtcttccgtcggtggggcccgaggacgcaggcgactatgtgtgcagagctgaggctgggctatcgggcctgcggggcggcgccgcggaggctcggctgactgtgaacgctcccccagtagtgaccgccctgcactctgcgcctgccttcctgaggggccctgctcgcctccagtgtctggttttcgcctctcccgccccagatgccgtggtctggtcttgggatgagggcttcctggaggcggggtcgcagggccggttcctggtggagacattccctgccccagagagccgcgggggactgggtccgggcctgatctctgtgctacacatttcggggacccaggagtctgactttagcaggagctttaactgcagtgcccggaaccggctgggcgagggaggtgcccaggccagcctgggccgtagagacttgctgcccactgtgcggatagtggccggagtggccgctgccaccacaactctccttatggtcatcactggggtggccctctgctgctggcgccacagcaaggcctcagcctctttctccgagcaaaagaacctgatgcgaatccctggcagcagcgacggctccagttcacgaggtcctgaagaagaggagacaggcagccgcgaggaccggggccccattgtgcacactgaccacagtgatctggttctggaggaggaagggactctggagaccaaggacccaaccaacggttactacaaggtccgaggagtcagtgtgagcctgagccttggcgaagcccctggaggaggtctcttcctgccaccaccctccccccttgggcccccagggacccctaccttctatgacttcaacccacacctgggcatggtccccccctgcagactttacagagccagggcaggctatctcaccacaccccaccctcgagctttcaccagctacatcaaacccacatcctttgggcccccagatctggcccccgggactccccccttcccatatgctgccttccccacacctagccacccgcgtctccagactcacgtgtgacatctttccaatggaagagtcctgggatctccaacttgccataatggattgttctgatttctgaggagccaggacaagttggcgaccttactcctccaaaactgaacacaaggggagggaaagatcattacatttgtcaggagcatttgtatacagtcagctcagccaaaggagatgccccaagtgggagcaacatggccacccaatatgcccacctattccccggtgtaaaagagattcaagatggcaggtaggccctttgaggagagatggggacagggcagtgggtgttgggagtttggggccgggatggaagttgtttctagccactgaaagaagatatttcaagatgaccatctgcattgagaggaaaggtagcataggatagatgaagatgaagagcataccaggccccaccctggctctccctgaggggaactttgctcggccaatggaaatgcagccaagatggccatatactccctaggaacccaagatggccaccatcttgattttactttccttaaagactcagaaagacttggacccaaggagtggggatacagtgagaattaccactgttggggcaaaatattgggataaaaatatttatgtttaataataaaaaaaagtcaaagaggaaaaaaaaaaaaaaaaaaaaaaaaaaaaaaaaaaaaaaaaaaaaaaaaaaaaaaaa

SEQ ID NO: 36 - protein, 688 aa, Homo sapiens
MLRMRVPALLVLLFCFRGRAGPSPHFLQQPEDLVVLLGEEARLPCALGAYWGLVQWTKSGLALGGQRDLPGWSRYWISGNAANGQHDLHIRPVELEDEASYECQATQAGLRSRPAQLHVLVPPEAPQVLGGPSVSLVAGVPANLTCRSRGDARPTPELLWFRDGVLLDGATFHQTLLKEGTPGSVESTLTLPTGRDTAITLSLQYPPEVTLSASPHTVQEGEKVIFLCQATAQPPVTGYRWAKGGSPVLGARGPRLEVVADASFLTEPVSCEVSNAVGSANRSTALDVLFGPILQAKPEPVSVDVGEDASFSCAWRGNPLPRVTWTRRGGAQVLGSGATLRLPSVGPEDAGDYVCRAEAGLSGLRGGAAEARLTVNAPPVVTALHSAPAFLRGPARLQCLVFASPAPDAVVWSWDEGFLEAGSQGRFLVETFPAPESRGGLGPGLISVLHISGTQESDFSRSFNCSARNRLGEGGAQASLGRRDLLPTVRIVAGVAAATTTLLMVITGVALCCWRHSKASASFSEQKNLMRIPGSSDGSSSRGPEEEETGSREDRGPIVHTDHSDLVLEEEGTLETKDPTNGYYKVRGVSVSLSLGEAPGGGLFLPPPSPLGPPGTPTFYDFNPHLGMVPPCRLYRARAGYLTTPHPRAFTSYIKPTSFGPPDLAPGTPPFPYAAFPTPSHPRLQTHV

SEQ ID NO: 37 - DNA, 2591 bp, Pan troglodytes
atgctcaggatgcgggtccccgccctcctcgtcctcctcttctgcttcagagggagcgcaggcccatcgccccatttcctgcaacagccagaggacctggtggtgctgctgggggaggaagcccggctgccgtgtgctctgggcgcctactgggggctagttcagtggactaagagtgggctggccctagggggccaaagggacctaccagggtggtcccggtactggatatcagggaatgcagccaatggccagcatgacctccacattaggcccgtggagctagaggatgaagcatcatatgaatgtcaggctacacaagcaggcctccgctccagaccagcccaactgcacgtgctggtacccccagaagccccccaggtgctgggcggcccctctgtgtctctggttgctggagttcctgcgaacctgacatgtcggagccgtggggatgcccgccctacccctgaattgctgtggttccgagatggggtcctgttggatggaaccaccttccatcagaccctgctgaaggaagggacccctgggtcagtggagagcaccttaaccctgacccctttcagccatgatgatggagccacctttgtctgccgggcccggagccaggccctgccctcaggaagagacacggctatcacactgagcctgcagtaccccccagaggtgactctgtctgcttcgccacacactgtgcaggagggagagaaggtcattttcctgtgccaggccacagaccagcctcctgtcacaggctacagggaggacgaagacccagaatccaccacaatggggaccaggcttcctacaaatccggcttctgacgccccttccctgtcgcaggtgctgggctctggagccacactgcatcttccgtcggtagggcccgaggacgcaggcgactatgtgtgcagagctgagcctgggctatcgggcctggggggcggcgccgcggaggctcggctgactgtgaacgctcccccagtagtgaccgccctgcactctgcgcctgccttcctgaggggccctgctcgcctccagtgtctggttttcgcctctcccgccccagatgccgtggtctggtcttgggatgagggcttcctggaggcggggtcgcagggccggttcctggtggagacattccctgccccagagagccgcgggggactgggtccgggcctgatctctgtgctacacatttcggggacccaggagtctgactttagcaggagctttaactgcagtgcccggaaccggctgggcgagggaggtgcccaggccagcctgggccgtagagacttgctgcccactgtgcggatagtggccggagtggccgctgccaccacgactctccttatggtcatcactggggtggccctctgctgctggcgccacagcaaggcctcagcctctttctccgagcaaaagaacctgatgcgaatccctggcagcagcgacggctccagttcgcgaggtcctgaagaagaggagacaggcagccgcgaggaccggggccccattgtgcacactgaccacagtgatctggttctggaggaggaagggactctggagaccaaggacccaaccaacggttactacaaggtccgaggagtcagtgtgagcctgagccttggcgaagcccctggaggaggcctcttcctgccaccaccctccccccttgggcccccagggacccctaccttctatgacttcaacccacacctgggcatggtccccccctgcagactttacagagccagggcaggctatctcaccacaccccaccctcgagctttcaccagctacatcaaacccacatcctttgggcccccagatctggcccccgggactccccccttcccatgtgctgccttccccacacctagccacccgcgtctccagactcatgtgtgacatctttccagtggaagagtcctgggatctccaacttgccataatggattgttctgatttctgaggagccaggacaagttggcgaccttactcctccaaaactgaacacagggggagggaaagatcattacatttgtcaggagcatttgtgtacagtcagctcagccaaaggagatgccccaagtgggagcaacgtggccacccaatatgcccacctattccccggtgtaaaagagattcaagatggcaggtaggccctttgaggagagatggggacagggcagtgggtgttgggagtttggggccgggatggaagttgtttctagccactgaaagaagatatttcaagatgaccatctgcattgagaggaaaggtagcataggatagatgaagatgaagagcataccaggccccaccctggctctccctgaggggaattttgctcggccaatggaaatgcagccaagatggccatatactccctaggaacccaagatggccaccatcttgattttactttccttaaagactcagaaagacttggactcaaggagtggggatacagtgagaattaccactgttggggcaaaggggattcttgggataaaaatatttatgtttaataataaaaaaaagtcaaagaggcaagtgtgtctta

SEQ ID NO: 38 - protein, 643 aa, Pan troglodytes
MLRMRVPALLVLLFCFRGSAGPSPHFLQQPEDLVVLLGEEARLPCALGAYWGLVQWTKSGLALGGQRDLPGWSRYWISGNAANGQHDLHIRPVELEDEASYECQATQAGLRSRPAQLHVLVPPEAPQVLGGPSVSLVAGVPANLTCRSRGDARPTPELLWFRDGVLLDGTTFHQTLLKEGTPGSVESTLTLTPFSHDDGATFVCRARSQALPSGRDTAITLSLQYPPEVTLSASPHTVQEGEKVIFLCQATDQPPVTGYREDEDPESTTMGTRLPTNPASDAPSLSQVLGSGATLHLPSVGPEDAGDYVCRAEPGLSGLGGGAAEARLTVNAPPVVTALHSAPAFLRGPARLQCLVFASPAPDAVVWSWDEGFLEAGSQGRFLVETFPAPESRGGLGPGLISVLHISGTQESDFSRSFNCSARNRLGEGGAQASLGRRDLLPTVRIVAGVAAATTTLLMVITGVALCCWRHSKASASFSEQKNLMRIPGSSDGSSSRGPEEEETGSREDRGPIVHTDHSDLVLEEEGTLETKDPTNGYYKVRGVSVSLSLGEAPGGGLFLPPPSPLGPPGTPTFYDFNPHLGMVPPCRLYRARAGYLTTPHPRAFTSYIKPTSFGPPDLAPGTPPFPCAAFPTPSHPRLQTHV

SEQ ID NO: 39 - DNA, 3019 bp, Bos taurus
atgggaagagggcgggtctcccagggggcgggccggggcggggcgagtggaaaccagccctccagggtctctccgagacctggcgcgcctaactggcaggcgtttcagagcggcggagtctgcggcggagcagacgaggggacctccaggtcagagagaggggagcttctagcctggggcgaagagtcaagagcggaggggactccaggccagagtggtgggaagtgtgctccgaggaaggaactggcagggaagccagtgcaacagcaagtctgctggatcttgggcgacacacttaggatgctcgtgcccgtactcctcgtcctctccttctgcctcagggggcgtgcaggcctggcaccccactttctgcaacagccagaggaccaggtagttctgttgggagacgaggcccgactgccctgtgccctgggcgactaccggggactggttcagtggactaaggacgggctggctctagggggcgaaagggaccttccaggctggtcccggtactggatatcagggaatgcagcaagtggccagcatgacctccacattagacccgtggagctagaggatcaagcatcgtatgaatgtcaagcgacacaagccggcctccgctctcgaccagcccaactgcatgtgctggtgcctccagaccccccccaggtgctgggcggcccctctgtgtctctggttgctggggttcctgcgaatctgacctgtcggagccgtggtgatgcccaccctacccctgagctgctgtggtttcgagatgggatccgactggacggggccatcttccgccagactcctttgaaggaaggaaccatcgggtcagtggagagcatcttgtctttgaccccttccagccatgatgatggagccaccttggtttgccgggcccggagccaggccctgcctgcagggaaggacacagctgtaacactaagcctgcagtaccccccagtggtgactctgtctgcagaaccacagacagtgcaggagggagagaaggtcactttcctatgccaggccacagcccagcctcctgtcaccggctatagatgggcaaaggggggctccccggtgctcggggcccgagggccaatgctggagatagtggctgacgcttcattcctgactgcgccggtgtcctgcgaggtcagcaatgcagtgggtagcgccaaccgcagcacagcgctggacgtgcagtttgggccgattctgcaggcaaagccgaaagccttgtcggtggacgtaggagaagacgcctccttcagctgtgtctggcgcgggaatccgctcccacgggtaacctggacccgccgcggggacgcacaggtgctggcctccgggcccacgctgcgtcttcccgcggtggggcccgaggatgcaggcgactacgtgtgcagggccgagccggggctctcaggccggggcggtggctctgcagaagctaggctgactgtgaacgctcccccagtagtgaccgctctgcactctgcgcccgccttcctgaggggccccgcccgcctccagtgtctagtcttcgcttcccccaccccagaagcagtggtctggtcttgggatgagggcttcctgacggcggggtcacggggtcggttcctggtggagactttcccagccccagagggcctcaagggacagggtccaggcctgatctccgtgctacacatttcggggacccaggagtccgactttcaccggggcttcaactgcactgcccggaaccgattgggtgagggaggcacccaggtcagcctgggccgtagagatttgctgcccactgtgcggattgtggccggagtggccgccatggccatgactctccttatgatcatcactggggtggccctctgctgctggcgccatggcaggggtcagtcctctttctccaagcaaaagaacctggtgcgaatcccagggagcagtgatggctccagttccaggggccccgaggaggagacaggcatcagcaaagaccagggccccatcgtgcacacggaccacagtgacctggttctggatgaggagggggctctggagaccaaggacccaaccaatggttactacaaggtccgaggagtcagtgtgagcctgagccttggagaagcccccggaggaggtctcttcctaccaccctcctcccctcttggacctccaggaacccctaccttctatgacttcaatccacatcttggcatggtccctccctgcagactatatagatcccgggcaggctatctcaccacaccccatcctcgagcttttaccagctacatcaaacccacatcctttggacccccagatctggcccccagcactccccccttcccatatgctgccttccccacgcccagccacccacgtctccagactcatgtgtgacatctctccagctgaagagtcctgggatcttcaacttgcacaatggattgtcctgatttctgagtaaccagaacaaactggcaaccctatccttccaaaattgaaccttgaggggagggaaagatcattgcagttgcctgggtcattgcgagaaagccactgggcttgcctcttcctctgtataaaagagattcaagagggcagatgggccctttgcagagggatggggacaggggttatgggagttagggacagagaaggaaattgcttccaaacattgaaagaagagatttcaagatggccacccacattaagaagaaaggcaacataaagcagatcaagatacaggacctgtccttgctatccctgaggggtactttgtttcacccatggaattgctgccaaaatggccattcattccctgggaatccaagatggccaccatcttgattctttctaccttccttaaaggccctggaagaaccggacccaaggagtaaggacagggtgaaagctgttgtggtggtgggaagggggaaaggagtggtgttggaatctgggaatgagaatatttatgttcaattaaaaaaaaaaaaagttgcaa

SEQ ID NO: 40 - protein, 804 aa, Bos taurus
MGRGRVSQGAGRGGASGNQPSRVSPRPGAPNWQAFQSGGVCGGADEGTSRSERGELLAWGEESRAEGTPGQSGGKCAPRKELAGKPVQQQVCWILGDTLRMLVPVLLVLSFCLRGRAGLAPHFLQQPEDQVVLLGDEARLPCALGDYRGLVQWTKDGLALGGERDLPGWSRYWISGNAASGQHDLHIRPVELEDQASYECQATQAGLRSRPAQLHVLVPPDPPQVLGGPSVSLVAGVPANLTCRSRGDAHPTPELLWFRDGIRLDGAIFRQTPLKEGTIGSVESILSLTPSSHDDGATLVCRARSQALPAGKDTAVTLSLQYPPVVTLSAEPQTVQEGEKVTFLCQATAQPPVTGYRWAKGGSPVLGARGPMLEIVADASFLTAPVSCEVSNAVGSANRSTALDVQFGPILQAKPKALSVDVGEDASFSCVWRGNPLPRVTWTRRGDAQVLASGPTLRLPAVGPEDAGDYVCRAEPGLSGRGGGSAEARLTVNAPPVVTALHSAPAFLRGPARLQCLVFASPTPEAVVWSWDEGFLTAGSRGRFLVETFPAPEGLKGQGPGLISVLHISGTQESDFHRGFNCTARNRLGEGGTQVSLGRRDLLPTVRIVAGVAAMAMTLLMIITGVALCCWRHGRGQSSFSKQKNLVRIPGSSDGSSSRGPEEETGISKDQGPIVHTDHSDLVLDEEGALETKDPTNGYYKVRGVSVSLSLGEAPGGGLFLPPSSPLGPPGTPTFYDFNPHLGMVPPCRLYRSRAGYLTTPHPRAFTSYIKPTSFGPPDLAPSTPPFPYAAFPTPSHPRLQTHV

SEQ ID NO: 41 - DNA, 291 bp, Artificial
  an artificially synthesized sequence
aagctttctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagactcgag

SEQ ID NO: 42 - DNA, 32 bp, Artificial
  an artificially synthesized sequence
gtaccactagtgctagcacgcgtgcggccgcg

SEQ ID NO: 43 - DNA, 32 bp, Artificial
  an artificially synthesized sequence
gatccgcggccgcacgcgtgctagcactagtg

SEQ ID NO: 44 - DNA, 3261 bp, Mus musculus
atcgattaggagcctatggtggcacttgtgagcaccagcagcttgtttgttgctgtcttccctgagcctctctctgtgtgccctgcttttctgttctttcaatctctttctaactcgcccttctctcttccctccctccctccctcccttccttctttctcttatcttcctaccattgtctgtgactgtcgcagtctttctgtcccggcatcgccttttctgcccttttctccccctttctcccccgttaacagttctttctctgagctctaaccccaagaccttaaaaatgcccccttctctctgtttttctttactatcacctccctctcacccactcacaggtactcaaagagcaccggctgctctaagtccagcaggttcacgaacccaaagcagtcagccacagcactgagcattgcttggacccagtgtgaactcattctgctgcttcccccaagtccttcctgagtctgcccagccgggcaatgaaatcccctgtcagcacatgccaggaacattctttggtgcctccccagcccgggtgaccccaaggccccgggagagcatttctgatcagggccatctccagctcaggatggaacgcagagctaggcctgtgtgcgtgggaaggctacaaccccccccccaggacatccttgctctttgcagcccagactgtggcatgtaccgcacccctgctggtgaatatgcaggagtgtgttgttaaaccaagagagtatgggacagagagaagccacttctgtgggtatgggtttcagagtgtgcgtgttccaggggagacgagagcagattttcatctgtggacgtatttggagtgtctcatatgggtgtgcacagagcatccagacagtcagcaggactgtgtccgcagacctcagtgtgtggaaacctggtgtgctcagacgcagataaacatagggtggggggtccctgggagctgaagcacctgaatccctgggccatgtgagggccttctgtttcctcaactcctccatcttcccttgtgtgtgtgtgtgtgtgtgtgtgtatgtcttgctaggatgatttatcttcttattcttctccacgtctttaattccatctcccccctcctcctcctcctccattcctacccccttctcattttctgtcactcttgctgccccctctgtgtctggtccttgaattttagaagcatatgttgactggcctgccctcccaacctgggtgcacagatgaattcgcctgggataagcccctgctgcctgcttatctcctgcaggaattgcaactgttctcttttgccccatattccctacaaacctctcacctcccggctgcttcttctccaggaggcagtccaggcatctggcctgagcccccctcacagctctctaccaagggtgagccctctggtctttctttcccacccagccccattattgattcattacccttggtccacattcccatctccaaacctatcaacacctcctgggcaatggattgcatacagaccaagccagggagtctgctatggggcctttgccagatggtatctttgggggccaaggtgtagagctgtcagttgctggagcctttaattagtgcaaaccttctacactcacctttggctgttttccttctctgaatgctctggggatcccaggctctccatttttccatcccgtggccccagagtgccaggaaagggaagctgtgtcaggtgtctggaaaaacagcctctcacctgacttcctcccggggacttaggagtcctgggctcaagtctgtcctatttcaaaactcagaagccaggagttcagatttaggtgtgaggcctcctaggactctcttttccaggcccccagcctgcaatccctccacttctacaggactcagcacagctgttcagtctgttggttttcccatcctcgggggccctagggggtgagtgagaacgagactggatgtcaaatccagtagctttaagacttaagaccagacattctgaaactggctcaactccaccacactgcagcctgcaaggcttctcagccagcagagaagtctgaactatccccctggggcaagtccaaccctctgatatttagaagtaccagtctgagccccaaatgtcatttatttctgagaggtccaggaatgtcagacccggggtctcaggcccccaacctccccccctccagccccttcagtgagctcagggtccctcccacctgctctgccagctgcactgcgtgggaacgcccagctgggctgcaccggagctgtcaggacaagctgtgcggttcccagcctccctccctgcctgccagagccagggcactgctgcctcccagccgtcgcccggcaaccactcaccactcggatgggccagggactgctgggtggagagaggggacgggtgggtgggtactgctgggtcctgggaaaggaggaactcctggaggggagggggcgggctagattcctagatcttaaggtacgtgtatggcttgcagggataacagagcagcagggagtattttgggaaatagggagtatttgggaaatagggagcagaaaccctcttctctcaaggctcctaaatggtccttcagcacctgggtgccctctctccgacccgcaggcccacgggagcctgagctccgcctccccagggcgcggaagctggcgaagccccagggattcccatttatagcttggtttccactcagctcagtccctccaggactcgggctgagcaagtttcttccattcccttctctcctccctccacccccttctcctcctccttctccttcttttcttcctcctcattcccgcctccccttcaacctcagcagggtgcaggtgtccaactcgaacaagggccccaacttggactcagatgttcccactctcagaccccctgataatgccgtccgcacacaaggcgcgggagtttctcaatgggaagaggccgggactctaggaggcggggcgaataggattcctcccgcctagtgggtccctcgcagtcctagggttgcaacccttgagcggtagagaacaccggagactgcggatgagccagatttcggggacataaaatcttccagcccggagagaattgtgtgcagagaggggctccagtccagcgtggtgtgagaggcgtgctatcaagaaagaagttggaggggaaccagtgcaaccctaactctgcgagatcttggggtacacacactcgggggtacc

SEQ ID NO: 45 - DNA, 798 bp, Mus musculus
ggtaccatgccgcgctccttcctggtggattcccttgtgctgcgggaagccagcgacaagaaggctccggagggcagcccgccaccgctcttcccctacgcggtcccgccgccgcacgcgctccacggcctctcgccgggcgcctgccacgcgcgcaaggccggcttgctgtgcgtgtgtcccctctgtgtcaccgcttcgcagctgcacgggccccccgggccgccggcactgccgctactcaaggcgtccttccctcccttcggatcgcagtactgccacgcacccctgggccgccagcactccgtgtcccctggagtcgcccacggcccggccgcggccgcagcagctgctgcactctaccagacctcctacccgctgccggatcccagacagtttcactgcatctctgtggacagcagctcgaaccagctgcccagcagcaagaggatgcggacggcgttcaccagcacacagctcctggagctggagcgagagttcgcctccaacatgtacctctcccgcctgcggcgcatcgagatcgcgacctatctgaacctgtccgagaagcaggtgaagatctggtttcagaaccgccgggtgaagcacaagaaagaaggcaaaggcagtaaccaccgcggcggagctggggcgggggccggcgggggcgcaccgcaaggctgcaagtgctcttcgctctcctcagccaaatgctcagaggacgacgacgaattgcccatgtctccatcttcctccgggaaggatgacagagatctcacagtcactccgtaggtcgac

SEQ ID NO: 46 - DNA, 4828 bp, Homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgccggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgcctttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaattttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccataaaaaaaaaaaaa

SEQ ID NO: 47 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 48 - DNA, 4413 bp, Mus musculus
actggtgtgggagccgcggcgcactactgagttcccaagaacttctgctagactcctgcccggcctaacccggccctgcccgaccgcacccgagctcagtgtttgctcggcgtctgccgggtccgccatgggagcccggtgccgcagcttttccgcgctcctgctcctgctgcaggtctcctcatggctttgccaggagctggagcctgagtcctgcagtcccggcttcagttccgaggtctacaccttcccggtgccggagaggcacctggagagaggccatgtcctgggcagagtgagatttgaaggatgcactggccggccaaggacagccttcttttcggaagactcccgattcaaagtggcgacagacggcaccatcacagtgaagcggcatctaaagctccacaagctggagaccagtttcctcgtccgcgcccgggactccagtcatagggagctgtctaccaaagtgacgctgaagtccatggggcaccaccatcaccggcaccaccaccgcgaccctgcctctgaatccaacccagagctgctcatgtttcccagcgtgtacccaggtctcagaagacagaaacgagactgggtcatccctcccatcagctgccccgaaaatgaaaagggcgaatttccaaagaacctggttcagatcaaatccaacagggacaaagaaacaaaggttttctacagcatcaccggccaaggagctgacaaaccccccgttggcgttttcatcattgagagggagacaggctggctgaaagtgacacagcctctggatagagaagccattgccaagtacatcctctattctcatgccgtgtcatcaaatggggaagcggtggaggatcccatggagatagtgatcacagtgacagatcagaatgacaacaggccagagtttacccagccggtctttgagggattcgttgcagaaggcgctgttccaggaacctccgtgatgaaggtctcagccaccgatgcagacgatgacgtcaacacctacaacgctgccatcgcctacaccatcgtcagccaggatcctgagctgcctcacaaaaacatgttcactgtcaatagggacaccggggtcatcagtgtgctcacctctgggctggaccgagagagttaccctacatacactctggtggttcaggctgctgaccttcaaggtgaaggcttgagcacaacagccaaggctgtgatcactgtcaaggatattaatgacaacgctcctgtcttcaacccaagcacgtatcagggtcaagtgcctgagaatgaggtcaatgcccggatcgccacactcaaagtgaccgatgatgatgcccccaacactccggcgtggaaagctgtgtacaccgtagtcaacgatcctgaccagcagttcgttgttgtcacagaccccacgaccaatgatggcattttgaaaacagccaagggcttggattttgaggccaagcagcaatacatccttcatgtgagagtggagaacgaggaaccctttgaggggtctcttgtcccttccacagccactgtcactgtggacgtggtagacgtgaatgaagcccccatctttatgcctgcggagaggagagtcgaagtgcccgaagactttggtgtgggtcaggaaatcacatcttataccgctcgagagccggacacgttcatggatcagaagatcacgtatcggatttggagggacactgccaactggctggagattaacccagagactggtgccattttcacgcgcgctgagatggacagagaagacgctgagcatgtgaagaacagcacatatgtagctctcatcatcgccacagatgatggttcacccattgccactggcacgggcactcttctcctggtcctgttagacgtcaatgacaacgctcccatcccagaacctcgaaacatgcagttctgccagaggaacccacagcctcatatcatcaccatcttggatccagaccttccccccaacacgtccccctttactgctgagctaacccatggggccagcgtcaactggaccattgagtataatgacgcagctcaagaatctctcattttgcaaccaagaaaggacttagagattggcgaatacaaaatccatctcaagctcgcggataaccagaacaaagaccaggtgaccacgttggacgtccatgtgtgtgactgtgaagggacggtcaacaactgcatgaaggcgggaatcgtggcagcaggattgcaagttcctgccatcctcggaatccttggagggatcctcgccctgctgattctgatcctgctgctcctactgtttctacggaggagaacggtggtcaaagagcccctgctgccaccagatgatgatacccgggacaatgtgtattactatgatgaagaaggaggtggagaagaagaccaggactttgatttgagccagctgcacaggggcctggatgcccgaccggaagtgactcgaaatgatgtggctcccaccctcatgagcgtgccccagtatcgtccccgtcctgccaatcctgatgaaattggaaacttcatcgatgaaaacctgaaggcagccgacagcgaccccacggcacccccttacgactctctgttggtgttcgattacgagggcagtggttctgaagccgctagcctgagctcactgaactcctctgagtcggatcaggaccaggactacgattatctgaacgagtggggcaaccgattcaagaagctggcggacatgtacggcggtggtgaggacgactaggggactagcaagtctcccccgtgtggcaccatgggagatgcagaataattatatcagtggtctttcagctccttccctgagtgtgtagaagagagactgatctgagaagtgtgcagattgcatagtggtctcactctccctactggactgtctgtgttaggatggttttcactgattgttgaaatctttttttattttttatttttacagtgctgagatataaactgtgcctttttttgtttgtttgtttctgtttttgttcttttgagctatgatctgccccagacacaacagccccaagcccctcacacctcactaattttttacattgtgtacttgccctcaattaccatgtttgctgtattctaatagtcactcatgttcctgaattctgttgccctgcccaggtgatattctaggatgcagaaatgcctgggcccttttatggtgagagacaggtatcttggtgtgggtgcaactgcgctggatagtgtgtgtgttcccaagatctttcgtggtattccctctccacctccagagaactcatttacagtggcattccttgttcggctatgtgtctggggcagaacaaaaaaaagggaccactatgcatgctgcacacgtctcagattcttaggtacacacctgattcttaggtgcatgccatagtgggatatgttgctttgatcagaacctgcagggaggttttcgggcaccacttaagtttcttggcgtttctttcaaaccaaaactaaagaatggttgttctctgagagagactggagtgccaccaccaaagacagaggagagaaaaggagagaaaccaaacttggggacagcaacatcagcgaacccggctagttggcacaccgatggtgagggtacacaggcggtgagacctatcccacaagatttctggaagactaggcttatctcaaccaatgttttctggctggaatctttgtccatgtattcctgaagcccaggaaatgcacccctccaatgcctgctcttgatggtagctacagaaaatgctggccgatttaaacccaagttgcccagttctgagtagaaaactgagactatgctgtgtgtggcggcgcgcacctttaagcccagcactcaggaggcagaggcagtcagatctccctgagttcgaggccaacctggtatatatagtatagtaagcgaattctaggacagccagggctacacagagaaaccctgtctctgcaaaccaaaagagaaaactgagaatattacaaattgtgcattttctcaggaagcaggaagagaacattctaacgggaaaaaggagacaagacctttgagagttttcattcaaaatgcaaatctcagctttttgataaccactggaaagaattttattgaaagttctgtacttacctaactttggaagaaaatgatgaccacaatcaactgtgagaactgttgatttctctgtagtttaatcatgtaatgttgctagagtgacctttgtatgtagtttgagtgtatgtgtgtgggtgctgataattttgtattttgtggggggtggaaaaggtaagccattgaaaccgttctctaagatgcatttttatgaattttattaaagagttttgttaaactgt

SEQ ID NO: 49 - protein, 884 aa, Mus musculus
MGARCRSFSALLLLLQVSSWLCQELEPESCSPGFSSEVYTFPVPERHLERGHVLGRVRFEGCTGRPRTAFFSEDSRFKVATDGTITVKRHLKLHKLETSFLVRARDSSHRELSTKVTLKSMGHHHHRHHHRDPASESNPELLMFPSVYPGLRRQKRDWVIPPISCPENEKGEFPKNLVQIKSNRDKETKVFYSITGQGADKPPVGVFIIERETGWLKVTQPLDREAIAKYILYSHAVSSNGEAVEDPMEIVITVTDQNDNRPEFTQPVFEGFVAEGAVPGTSVMKVSATDADDDVNTYNAAIAYTIVSQDPELPHKNMFTVNRDTGVISVLTSGLDRESYPTYTLVVQAADLQGEGLSTTAKAVITVKDINDNAPVFNPSTYQGQVPENEVNARIATLKVTDDDAPNTPAWKAVYTVVNDPDQQFVVVTDPTTNDGILKTAKGLDFEAKQQYILHVRVENEEPFEGSLVPSTATVTVDVVDVNEAPIFMPAERRVEVPEDFGVGQEITSYTAREPDTFMDQKITYRIWRDTANWLEINPETGAIFTRAEMDREDAEHVKNSTYVALIIATDDGSPIATGTGTLLLVLLDVNDNAPIPEPRNMQFCQRNPQPHIITILDPDLPPNTSPFTAELTHGASVNWTIEYNDAAQESLILQPRKDLEIGEYKIHLKLADNQNKDQVTTLDVHVCDCEGTVNNCMKAGIVAAGLQVPAILGILGGILALLILILLLLLFLRRRTVVKEPLLPPDDDTRDNVYYYDEEGGGEEDQDFDLSQLHRGLDARPEVTRNDVAPTLMSVPQYRPRPANPDEIGNFIDENLKAADSDPTAPPYDSLLVFDYEGSGSEAASLSSLNSSESDQDQDYDYLNEWGNRFKKLADMYGGGEDD

SEQ ID NO: 50 - DNA, 4396 bp, Rattus norvegicus
ctggtgtgggagccgcggcgcactactgagttctcaagaacttctgctagacttcagcccggcctaacccggctctgcccgaccgcacccgagctcagtgtttgctcggcgtttgcccggccagccatgggagcccggtgccgcagcttctccgcgctcctgctcctgctgcaggtctcctcgtggctttgtcagcagccggagtcggagtctgactcctgccgtcccggcttcagttccgaggtctacaccttcctggtgccggagaggcacctggagagaggccacatcctgggcagagtgaaatttgaaggatgcaccggccgtccaaggacagccttcttttctgaagactcccgattcaaagtgtctacagatggcgtcatcacagtcaaacggcatctaaagcttcacaagctggagaccagttttctcgtccatgcctgggactccagttacaggaagctttctaccaaagtgacactgaagtccctgggccaccaccaccaccggcatcaccacagagaccctgtctctgaatccaacccagagctgctcacgtttcccagctttcaccagggtctgagaagacagaaacgagactgggtcatccctcccatcaactgcccggaaaatcaaaagggcgaattcccccagcgactggttcagatcaaatccaacagggacaaagagacaacggttttctacagcatcaccggcccaggagctgacaaaccccctgttggcgttttcatcattgagagggagacaggctggctgaaagtgacgcagcctctggacagagaagccattgacaagtaccttctctactctcatgctgtgtcatcaaatggggaagccgtggaggatcccatggagatagtggtcacagtcacagatcagaatgacaacaggccagagtttatccaggaggtctttgagggatctgttgcagaaggcgctcttccaggaacctccgtgatgcaggtctcagccactgatgcagacgatgacataaacacctacaatgctgccatcgcctacaccatcctcagccaagatcctgagctgcctcacaaaaacatgttcactgtcaaccgggacactggggtcatcagtgtggtcacctccggactggaccgagagagttaccctacatatactctggtggttcaggctgccgaccttcaaggcgaaggcctaagcacaacagcaaaagctgtgatcactgtcaaggatattaatgacaacgctcccatcttcaacccaagcacgtaccagggtcaagtgcttgagaatgaggtcggtgcccgtattgccacactcaaggtgactgatgatgatgcccccaacactccagcgtggaatgctgtgtacaccgtagtcaatgatcctgatcatcagttcactgtcatcacagaccccaagaccaacgagggcattctgaaaacagccaagggcttggattttgaggccaagcagcagtacattctgcacgtgacagtggaaaatgaggagccctttgaggggtctcttgtcccttccacagccactgtcaccgtggatgtggtagacgtgaatgaagcccccatttttgtgcctgcggagaagagagtcgaggtgcctgaggactttggtgtgggtctggagatcgcatcttacactgcgcgagagccagacacattcatggaacagaagatcacgtatcggatttggagggacactgccaattggctggagattaacccagagactggggtcatttccactcgggctgagatggacagagaagattcggagcatgtgaagaacagcacgtatacagctctcatcattgccacagatgatggttcacccattgccactggcacagggactcttctcctggtcctgtcagacgtcaacgacaatgctcccatcccagaacctcgaaatatgcagttctgccagagaaacccgaagccccatgtcatcaccatcttggatccagaccttcccccaaacacatcccccttcactgcagagctcacccatggggccagcgtcaactggaccattgagtacaatgacgcagaacaagaatctctcattttgcaaccaagaaaggacttagagattggcgaatacaaaatcaatctcaagctctcggataaccagaataaagaccaggtgaccacgttggaggtccacgtgtgtgactgtgaagggaccgtcaacaactgcatgaaggcgatctccctggaagcaggattacaagttcccgccatccttggaatcctgggagggatcctggccctcctgattctgatcctcctgctcctactgtttctacggaggagaacggtggtcaaagagcccttgctgccaccagatgacgatacccgggacaatgtgtattactatgatgaagagggaggtggagaagaagaccaggactttgatttgagccagctgcacaggggccttgatgccagaccggaagtgattcgaaatgatgtggctcccaccctcatgagcatgccccagtatcgtccccgtccagccaatcctgatgaaatcgggaacttcatcgatgaaaacctgaaggcagcggacagtgaccccacagcgcccccttacgactctctgttggtgtttgactatgaggggagtggttctgaagctgcctccctgagctcgctgaactcctctgagtcagatcaggaccaggactacgattatctgaacgaatggggcaaccggttcaagaagctggccgatatgtatggtggcggcgaagaagactagagagtcgttcctgtgtggcaccatgggagatgcagaatcatgatgtcagtggtctttcagctccttccctgactttgtagaagagagactgatctgagaagtatgcagattgcatactggtcccactctacctaccagtctgtctgtgttaggagggttttcactggttgttggaatctttttctaaaatgtttttgtttttacagtgctgtgatgtgatgaactgtaccctctttttgtttttgttttgagctatgttctgctccggacacacagcccccaagcccttcacccctcactaatttttttacattgtatactttcactcaattaccatgtttatgttctgtattctaatagccactaagttcctgaattctgttgcctggcccaggtgctattctgtgacacagtagtgcctgggcccttttatggtaagagacaggtttcttggtgtgggtgcaactgagctggatagtgtatgtttcaaacacctttcctgtgttctctccccacctccagagtgtctttacttattcagctgtgtgtttggggcagaacaaaaaaataatgggaccactatgcaagctgcgaagattctaaggtgcacacctgattcttaggcagatgccatagtgagatatgttgctttggttctctatccaatgctgtgaccgggacctgcagcgaggttttcggacaccgtggtttcttgcgtttctttcaaaccagcagtaaaaaatggttttttctgagagagactggagtgccaccaccaaagatagaggagagaagccaagcttggggacagcaagcatgccagtgaacctgaccactgtcatgagtcatgtgggtggccacatgtccgtgaacctggccagttggcacactgatggtgagggtacaaggaggctagacctcgtcccacaaaatttctggaagaattagggttgtctcagccaatgtttcctagctggaatcctgtccatgtatgtgttcctgaagcccaggaaatacacccctctagtgcctgcttttgatggtagttatagaaaagaccggctgatttggacctgagttgcccaatcttaagtacaaatagaaaactgagactatgctgggtgtgttggtgcacgcctttaatcccggcactcgggaggcagagacagtctcagatctctctgagttcaaggtcagcctggtatagtaagtgaattccaggacagccagggctacacagaaactctgtcttggaaaaccaaaaaagaaaactgagaatattagagattgtgcattttctcagaaagcaggaagaaaacaccactctgatgggaaaagggaggcaaggcccttgagacttttcattgaaattgctgtactcacataattttggaagcaaatgatgactgcaatcaactgtgagaactgttggtttctctgtagtttaattgtctaatgttgatagcgtgccctttgtatgtagtttgagtgtatatgtgtgtgggtgctgataattttgtattttgtggggagtggaaaaggcaagcaatcggaactgttctctaagatgcatttttatgaattttattaaagagttttgttaaactgt

SEQ ID NO: 51 - protein, 886 aa, Rattus norvegicus
MGARCRSFSALLLLLQVSSWLCQQPESESDSCRPGFSSEVYTFLVPERHLERGHILGRVKFEGCTGRPRTAFFSEDSRFKVSTDGVITVKRHLKLHKLETSFLVHAWDSSYRKLSTKVTLKSLGHHHHRHHHRDPVSESNPELLTFPSFHQGLRRQKRDWVIPPINCPENQKGEFPQRLVQIKSNRDKETTVFYSITGPGADKPPVGVFIIERETGWLKVTQPLDREAIDKYLLYSHAVSSNGEAVEDPMEIVVTVTDQNDNRPEFIQEVFEGSVAEGALPGTSVMQVSATDADDDINTYNAAIAYTILSQDPELPHKNMFTVNRDTGVISVVTSGLDRESYPTYTLVVQAADLQGEGLSTTAKAVITVKDINDNAPIFNPSTYQGQVLENEVGARIATLKVTDDDAPNTPAWNAVYTVVNDPDHQFTVITDPKTNEGILKTAKGLDFEAKQQYILHVTVENEEPFEGSLVPSTATVTVDVVDVNEAPIFVPAEKRVEVPEDFGVGLEIASYTAREPDTFMEQKITYRIWRDTANWLEINPETGVISTRAEMDREDSEHVKNSTYTALIIATDDGSPIATGTGTLLLVLSDVNDNAPIPEPRNMQFCQRNPKPHVITILDPDLPPNTSPFTAELTHGASVNWTIEYNDAEQESLILQPRKDLEIGEYKINLKLSDNQNKDQVTTLEVHVCDCEGTVNNCMKAISLEAGLQVPAILGILGGILALLILILLLLLFLRRRTVVKEPLLPPDDDTRDNVYYYDEEGGGEEDQDFDLSQLHRGLDARPEVIRNDVAPTLMSMPQYRPRPANPDEIGNFIDENLKAADSDPTAPPYDSLLVFDYEGSGSEAASLSSLNSSESDQDQDYDYLNEWGNRFKKLADMYGGGEED

SEQ ID NO: 52 - DNA, 4877 bp, Pan troglodytes
agccaatcagcggtacggggggcggtgcctccggggctcacctggcggcagccgcgcaccctctctcagtggcgtcggaactgcaaagcacctgtgagcttgcggaagtcagttcagactccagcccgctccagcccggcccgacccgaccgcacccggcgcctgccctctctcggcgtccccggccagccatgggcccttggagccgcagcctctcagcgctgctgctgctgctgcaggtctcctcttggctctgccaggagccggagccctgccaccctggctttgacgccgagagctacacgttcacggtgccccggctccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctctgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcatgctgtgtcatccaacgggaatgccgttgaggatccaatggagattttgatcacggtgaccgatcagaatgacaacaaacccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctgcctgacaaaaatatgttcaccattaacaggaacacaggagtcatcagtgtggtcaccactgggctggaccgagagagtttccctacgtataccctggtggttcaagctgctgatcttcaaggtgaggggttaagcacaacagcaacagctgtgatcacagtcactgacaccaacgataaccctccgatcttcaatcccaccacgtacaagggtcaggtgcctgagaacgaggctaacgtcataatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtcggaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggatttggagcacgtgaagaacagcacgtacacagccctaatcatagctacagacaatggttctccagttgctactggaacagggacacttctgctgatcctgtctgatgtgaatgacaatgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgccggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagttgcacaggggcctggacgctcggcctgaagtgactcgtaacgacgttgcaccaaccctcatgagtgtcccccggtaccttccccgccctgccaatcccgatgaaattggaaattttattgatgaaaatctgaaagcggctgatactgaccccacagccccgccttatgattctcttctcgtgtttgactatgaaggaagcggttccgaagctgctagtctgagctccctgaactcctcagagtcagacaaagaccaggactatgactacttgaacgaatggggcaatcgcttcaagaagctggcggacatgtacggaggtggcgaggacgactaggggactcgagagaggcgggccctagacccgtgtgctgggaaatgcagaaatcatgttgctggtggtttttcagctcccttcccttgagatgagtttctggggaaaagaagagactggttggtgatgcagttagtatagctttatgctctctccactttatagctctaataagtgtgtgttagaaaagttttgacttatttcttgaagcttttttttttttcccatcactctacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgctcctgctcattgctacactggtgtgtccctctgccttttttttttttaagacagggtctcattctgtcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccattgcccaggcctgggatgcagtgatgtgatcatagctcactgtaacctcaaactctggggctcaagcaattctcccaccagcctcctttttatttttttgtacagatggggtcttgctatgttgcccatgctggtcttaaactcctggcctcaagcaatcctcctgccttggccccccaaagtgctgggattgcgggcatgagctgctgtgcccagcctccatgttttaatatcaactctccctcctcaattcagttgctttgcccaagataggagttctccgatgcagaaattattgggctcttttagggtaagaagctcgtgtcttcgtctggccacatcttgactaggtattgtctactctgaagacctttaatggcttccctctttcatctcctgagtatgtaacttgcaatgggcatctatccagtgacttgttctgagtaagtgtgttcgttaatgtttatttagctctgaagcaagagtgatatactccaggacttagaatagtgcctaaagtgctgcagccaaagacagagcggaactatgaaaattgggcttggagatggcaggagagcttgtcattgagcctggcaatttagcaaactgatgctgaggatgattgaggtgggtctacctcatctctgaaaattctggaaggaatggaggagtctcaacatgtgtttctgacacaggatctgtggtttgtactcaaaggccagaatccccaagtgcctgcttttgatgatgtctacagaaaatgctggctgagctgaacacatttgcccaattccagatgtgcatagaaaactgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagagttttattgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctctgggggtggaaaaggaaaacgattcaagctgagaaaagtattctcaaagatgcatttttataaattttattaaagaattttgttaaaccat

SEQ ID NO: 53 - protein, 882 aa, Pan troglodytes
MGPWSRSLSALLLLLQVSSWLCQEPEPCHPGFDAESYTFTVPRLHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVIITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDLEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 54 - DNA, 26 bp, Artificial
  an artificially synthesized sequence
cagctccacaacctacatcattccgt

SEQ ID NO: 55 - DNA, 12 bp, Artificial
  an artificially synthesized sequence
acggaatgatgt

SEQ ID NO: 56 - DNA, 26 bp, Artificial
  an artificially synthesized sequence
ctccaatgcctgctcttgatggtagc

SEQ ID NO: 57 - DNA, 26 bp, Artificial
  an artificially synthesized sequence
tctctgtgtagccctggctgtcctag

SEQ ID NO: 58 - DNA, 26 bp, Artificial
  an artificially synthesized sequence
cttcccgtatgctaccttgtctccac

SEQ ID NO: 59 - DNA, 26 bp, Artificial
  an artificially synthesized sequence
ccaacagtcctgcatgcttgtaatga